(19) United States
(12) Patent Application Publication
(10) Pub. No.: US 2015/0299182 A1
(43) Pub. Date: Oct. 22, 2015

(54) CRYSTALLINE FORMS OF (1S)-1-[5-(AMINO)-1,2,4-THIADIAZOL-3-YL]ETHANE-1,2-DIOL

(71) Applicants: Array BioPharma Inc., Boulder, CO (US); Amgen Inc., Thousand Oaks, CA (US)

(72) Inventors: Sylvie Asselin, Thousand Oaks, CA (US); Lisa Brett, Kirkland, WA (US); Ying Chen, Newbury Park, CA (US); Donald T. Corson, Boulder, CO (US); Andrew Cosbie, Ventura, CA (US); Robert Farrell, Thousand Oaks, CA (US); Indrani W. Gunawardana, Boulder, CO (US); Jinkun Huang, Thousand Oaks, CA (US); Jonathan W. Lane, Boulder, CO (US); Dennis Lei, West Covina, CA (US); Christopher M. Lindemann, Fort Collins, CO (US); Van Luu, Temecula, CA (US); Coralee G. Mannila, Boulder, CO (US); Robert Milburn, Thousand Oaks, CA (US); Henry Morrison, Moorpark, CA (US); Helming Tan, Thousand Oaks, CA (US); Jason Tedrow, Santa Monica, CA (US); Daniel J. Watson, Boulder, CO (US)

(73) Assignees: Amgen Inc., Thousand Oaks, CA (US); Array BioPharma Inc., Boulder, CO (US)

(21) Appl. No.: 14/441,133

(22) PCT Filed: Nov. 8, 2013
(86) PCT No.: PCT/US2013/069331

§ 371 Date: May 6, 2015

Related U.S. Application Data
(60) Provisional application No. 61/724,497, filed on Nov. 9, 2012.

(51) Int. Cl. C07D 417/14

(52) U.S. Cl. CPC C07D 417/14; C07B 2200/13

(57) ABSTRACT

The present invention relates to crystalline polymorph forms of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol, to pharmaceutical compositions comprising such crystalline polymorph forms, and to processes for preparing them. The invention further relates to methods of treatment of diabetes related disorders comprising administering such solid-state forms or compositions thereof to a subject, and to use of such crystalline polymorph forms in the manufacture of medicaments.

### FIELD OF THE INVENTION

The present invention relates to crystalline, polymorphic forms of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol, also known as AMG151 and ARRY-403, to pharmaceutical compositions comprising such crystalline polymorphic forms, and to processes for preparing them. The invention further relates to methods of treatment of diabetes related disorders comprising administering such solid-state forms or compositions thereof to a subject, and to use of such crystalline polymorphic forms in the manufacture of medicaments.

### BACKGROUND OF THE INVENTION

AMG151, also known as ARRY-403 and having the chemical name (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol, and its pharmaceutically acceptable salts, have a therapeutic effect in the treatment of diabetes. AMG151 is a glucose kinase activator which increases insulin production in pancreatic beta cells in a glucose dependent manner and increases glucose uptake in liver.

Treatment with AMG151 may be indicated in a very wide array of diabetes-related conditions and other disorders. Therefore, in the use of the thermodynamically stable free base forms, salt forms and co-crystals, a significant advance would be realized in treatment of diabetes-related conditions and disorders. The phase 1 clinical material was prepared as the mono-hydrochloride salt Form C. We have found that the mono-hydrochloride salt Form C was hygroscopic and had less preferred thermal stability characteristics.

In order to overcome this issue, we sought to identify a physical form with physical-chemical properties suitable for development.

### BRIEF DESCRIPTION OF THE INVENTION

Provided herein are crystalline free base forms of AMG151, crystalline hydrates of the AMG151 free base, crystalline solvates of AMG151 free base, crystalline salt forms of AMG151, and amorphous AMG151 free base.

Also provided herein is a crystalline polymorph of AMG151, Form C.

Also provided herein is a crystalline polymorph of AMG151, Form A.

Also provided herein is a crystalline polymorph of AMG151, Form F.

Also provided herein is a mixture of crystalline polymorph of AMG151, Form A and crystalline polymorph of AMG151, Form F.

Also provided herein is a pharmaceutical composition comprising a crystalline polymorph described herein and one or more pharmaceutically acceptable excipients.

Also provided herein is a method of treating diabetes in a patient, comprising administering a therapeutically effective amount of a crystalline polymorph described herein to a patient in need thereof.

Also provided herein is the use of a crystalline polymorph described herein in the manufacture of a medicament for the management or treatment of diabetes.

Also provided are processes for preparing a crystalline free base form of AMG151, a crystalline hydrate of the AMG151 free base, AMG151 crystalline salt forms, amorphous AMG151 free base, and a crystalline solvate of AMG151.

Also provided herein are processes for preparing AMG151.

Also provided herein are processes for preparing the crystalline polymorph of AMG151 freebase, substantially in the form of Form A.

Also provided herein are processes for preparing the crystalline polymorph of AMG151 monohydrate, Form C.

Also provided herein are processes for preparing the crystalline polymorph of AMG151 monohydrate, Form C with consistent particle size distribution.

### BRIEF DESCRIPTION OF THE DRAWINGS

FIG. 1 shows an X-Ray Powder Diffraction (XRPD) pattern of amorphous AMG151.

FIG. 2 shows a differential scanning calorimetry (DSC) thermogram of amorphous AMG151.

FIG. 3 shows an XRPD pattern for AMG151 dihydrochloride, Form B.

FIG. 4 shows a DSC thermogram and thermogravimetric analysis (TG or TGA) of AMG151 dihydrochloride salt Form B.

FIG. 5 shows a DSC thermogram and TGA of AMG151 dihydrochloride salt, Form A.

FIG. 6 shows an XRPD pattern for AMG151 dihydrochloride salt, Form A.

FIG. 7 shows an XRPD pattern for AMG151 fumarate solvate Form A.

FIG. 8 shows a DSC thermogram and thermogravimetric analysis (TG) of AMG151 fumarate solvate Form A.

FIG. 9 shows a DSC thermogram and TGA results for AMG151 hydrate Form C.

FIG. 10 shows an XRPD pattern for AMG151 hydrate Form C.

FIG. 11 shows a variable temperature XRPD pattern for AMG151 hydrate Form C.

FIG. 12 shows a moisture sorption curve for AMG151 hydrate Form C.

FIG. 13 shows an XRPD pattern for AMG151 free base Form A and AMG151 free base Form F.

FIG. 14. shows vapor sorption curves for AMG151 Form A and Form F.

FIG. 15. shows a DSC thermogram of AMG151 Form A and Form F.

FIG. 16. shows an XRPD pattern for AMG151 HCl salt Form A.

FIG. 17. shows a DSC and TGA curve for AMG151 HCl salt Form A.

FIG. 18. shows a vapor sorption curve for AMG151 HCl salt Form A.

FIG. 19. shows an XRPD pattern for AMG151 HCl salt Form B.

FIG. 20. shows DSC and TGA curves for AMG151 HCl salt Form B.

FIG. 21. shows a vapor sorption curve for AMG151 HCl salt Form B.

FIG. 22. shows an XRPD pattern for AMG151 HCl salt Form C.

FIG. 23. shows DSC and TGA curves for AMG151 HCl salt Form C.

FIG. 24. shows an XRPD pattern for AMG151 HCl salt Form D.

FIG. 25. shows DSC and TGA curves for AMG151 HCl salt Form D.

FIG. 26. shows an XRPD pattern for AMG151 phosphate salt Form A.

FIG. 27. shows DSC and TGA curves for AMG151 phosphate salt Form A.

FIG. 28. shows a vapor sorption curve for AMG151 phosphate salt Form A.

FIG. 29. shows a XRPD pattern for AMG151 phosphate salt Form B.

FIG. 30. shows DSC and TGA curves for AMG151 phosphate salt Form B.

FIG. 31. shows a vapor sorption curve for AMG151 phosphate salt Form B.

FIG. 32. shows an XRPD pattern for AMG151 phosphate salt Form C.

FIG. 33. shows DSC and TGA curves for AMG151 phosphate salt Form C.

FIG. 34. shows an XPRD pattern for AMG151 phosphate salt Form D.

FIG. 35. shows DSC and TGA curves for AMG151 phosphate salt Form D.

FIG. 36. shows a vapor sorption curve for AMG151 phosphate salt Form D.

FIG. 37. shows an XRPD pattern for AMG151 phosphate salt Form E.

FIG. 38. shows DSC and TGA curves for AMG151 phosphate salt Form E.

FIG. 39. shows a vapor sorption curve for AMG151 phosphate salt Form E.

FIG. 40. shows an XRPD pattern for AMG151 sulfate salt Form A.

FIG. 41. shows a DSC and TGA curves for AMG151 sulfate salt Form A.

FIG. 42. shows an XRPD pattern for AMG151 sulfate salt Form B.

FIG. 43. shows DSC and TGA curves for AMG151 sulfate salt Form B.

FIG. 44. shows an XRPD pattern for AMG151 methanesulfonic acid salt Form A.

FIG. 45. shows DSC and TGA curves for AMG151 methanesulfonic acid salt Form A.

FIG. 46. shows an XRPD pattern for AMG151 methanesulfonic acid salt Form B.

FIG. 47. shows a DSC curve for AMG151 methanesulfonic acid salt Form B.

FIG. 48. shows a Vapor Sorption curve for AMG151 methanesulfonic acid salt Form B.

FIG. 49. shows an XRPD pattern for AMG151 methanesulfonic acid salt Form C.

FIG. 50. shows a DSC and TGA curves for AMG151 methanesulfonic acid salt Form C.

FIG. 51. shows a vapor sorption curve for AMG151 methanesulfonic acid salt Form C.

FIG. 52. shows an XRPD pattern for AMG151 succinate Form A.

FIG. 53. shows a DSC curve for AMG151 succinate Form A.

FIG. 54. shows a vapor sorption curve for AMG151 fumarate Form A.

FIG. 55. shows an XRPD pattern for AMG151 free base Form G.

FIG. 56. shows a DSC curve for AMG151 free base Form G.

FIG. 57. shows DSC and TGA curves of process optimized lot of AMG151 fumarate.

FIG. 58. shows a solid state stability data on AMG151 free base Form F.

FIG. 59. shows a solid state stability data on AMG151 fumarate.

FIG. 60. shows a solid state stability data on AMG151 free base hydrate Form C.

FIG. 61. shows a solid state stability data on AMG151 phosphate.

FIG. 62. shows an XRPD pattern for AMG151 acetic acid solvate Form J.

FIG. 63. shows a DSC curve for AMG151 acetic acid solvate Form J.

FIG. 64. shows an XRPD pattern for AMG151 acetic acid solvate Form K.

FIG. 65. shows DSC and TGA curves for AMG151 acetic acid solvate Form K.

FIG. 66. Shows a DSC curve of AMG151 Form I.

FIG. 67. shows XRPD patterns for AMG151 Forms A, F and I.

FIG. 68 shows an XRPD pattern for AMG151 free base Form L.

FIG. 69 shows the DSC curve for AMG151 Form L.

FIG. 70 shows the DSC curve for AMG151 free base Form M.

FIG. 71 shows the XRPD pattern for AMG151 free base form M.

FIG. 72 shows the particle size distribution of the crystalline polymorph of AMG151 monohydrate, Form C, crystallized using sulfuric acid.

FIG. 73 shows the XRPD pattern for AMG151 Form A prepared by crystallization of AMG151 hydrate Form C.

### DETAILED DESCRIPTION OF THE INVENTION

U.S. Pat. Nos. 8,022,223 and 8,212,045 describe a family of substituted pyridines, including AMG151, and pharmaceutically acceptable salts thereof, as agents for treatment of diabetes. A need exists for new forms of AMG151, in particular thermodynamically stable forms suitable for preparing pharmaceutical compositions, including aqueous suspensions.

AMG151 free base is predisposed to polymorphism and an unsolvated free base Form A and free base Form F were identified. Free base Form F was found to have a similar melting temperature and similar solubility in several solvents as free base Form A. Although individually free base Form A and free base Form F are viable options, the similarity of polymorphs A and F resulted in a lack of phase control during scale up efforts even through the use of seeding to control forms. A mixture of free base Form A and free base Form F could be useful also, although not optimal.

Alternatively, a hydrate of the free base, Form C, was identified, which also displayed viable thermal behavior and physical properties.

Various crystalline salts of the free base were prepared and analyzed. Several salts, including other forms of an HCl salt, a bis-HCl salt, phosphate salt forms, sulfate salt forms salts, methanesulfonic acid salt forms, possess potential.

In addition, various co-crystals were discovered and identified as preferred crystalline material. Crystalline solvates were also identified, including an acetic acid solvate as well as some high temperature stable forms.

As a further alternative, amorphous AMG151 free base was prepared.

Also provided are processes for preparing a crystalline free base form of AMG151, a crystalline hydrate of the AMG151 free base, AMG151 crystalline salt forms, amorphous AMG151 free base, and a crystalline solvate of AMG151.

Also provided are processes for preparing the AMG151 crystalline drug substance of the invention. AMG151 crystalline drug substance or powder thereof, prepared according to such processes can be further formulated to provide a pharmaceutical dosage form.

In addition, processes for preparing AMG151 are provided.

The term “H” denotes a single hydrogen atom. This radical may be attached, for example, to an oxygen atom to form a hydroxyl radical.

Where the term “alkyl” is used, either alone or within other terms such as “haloalkyl” and “alkylamino”, it embraces linear or branched radicals having one to about twelve carbon atoms. More preferred alkyl radicals are “lower alkyl” radicals having one to about six carbon atoms. Examples of such radicals include methyl, ethyl, n-propyl, isopropyl, n-butyl, isobutyl, sec-butyl, tert-butyl, pentyl, isoamyl, hexyl and the like. Even more preferred are lower alkyl radicals having one or two carbon atoms. The term “alkylenyl” embraces bridging divalent alkyl radicals such as methylenyl and ethylenyl.

The term “aryl”, alone or in combination, means a carbocyclic aromatic system containing one or two rings wherein such rings may be attached together in a fused manner. The term “aryl” embraces aromatic radicals such as phenyl, naphthyl, indenyl, tetrahydronaphthyl, and indanyl. More preferred aryl is phenyl. Said “aryl” group may have 1 to 3 substituents such as lower alkyl, hydroxyl, halo, haloalkyl, nitro, cyano, alkoxy and lower alkylamino. Phenyl substituted with —O—CH2-O— forms the aryl benzodioxolyl substituent.

The term “heterocyclyl” embraces saturated, partially saturated and unsaturated heteroatom-containing ring radicals, where the heteroatoms may be selected from nitrogen, sulfur and oxygen. It does not include rings containing —O—O—, —O—S— or —S—S— portions. Said “heterocyclyl” group may have 1 to 3 substituents such as hydroxyl, Boc, halo, haloalkyl, cyano, lower alkyl, lower aralkyl, oxo, lower alkoxy, amino and lower alkylamino.

Examples of saturated heterocyclic radicals include saturated 3 to 6-membered heteromonocyclic groups containing 1 to 4 nitrogen atoms [e.g. pyrrolidinyl, imidazolidinyl, piperidinyl, pyrrolinyl, piperazinyl]; saturated 3 to 6-membered heteromonocyclic group containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of partially saturated heterocyclyl radicals include dihydrothienyl, dihydropyranyl, dihydrofuryl and dihydrothiazolyl.

Examples of unsaturated heterocyclic radicals, also termed “heteroaryl” radicals, include unsaturated 5 to 6 membered heteromonocyclyl group containing 1 to 4 nitrogen atoms, for example, pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated 5- to 6-membered heteromonocyclic group containing an oxygen atom, for example, pyranyl, 2-furyl, 3-furyl, etc.; unsaturated 5 to 6-membered heteromonocyclic group containing a sulfur atom, for example, 2-thienyl, 3-thienyl, etc.; unsaturated 5- to 6-membered heteromonocyclic group containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic group containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl].

The term also embraces radicals where heterocyclic radicals are fused/condensed with aryl radicals: unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo[1,5-b]pyridazinyl]; unsaturated condensed heterocyclic group containing 1 to 2 oxygen atoms and 1 to 3 nitrogen atoms [e.g. benzoxazolyl, benzoxadiazolyl]; unsaturated condensed heterocyclic group containing 1 to 2 sulfur atoms and 1 to 3 nitrogen atoms [e.g., benzothiazolyl, benzothiadiazolyl]; and saturated, partially unsaturated and unsaturated condensed heterocyclic group containing 1 to 2 oxygen or sulfur atoms [e.g. benzofuryl, benzothienyl, 2,3-dihydro-benzo[1,4]dioxinyl and dihydrobenzofuryl]. Preferred heterocyclic radicals include five to ten membered fused or unfused radicals. More preferred examples of heteroaryl radicals include quinolyl, isoquinolyl, imidazolyl, pyridyl, thienyl, thiazolyl, oxazolyl, furyl, and pyrazinyl. Other preferred heteroaryl radicals are 5- or 6-membered heteroaryl, containing one or two heteroatoms selected from sulfur, nitrogen and oxygen, selected from thienyl, furyl, pyrrolyl, indazolyl, pyrazolyl, oxazolyl, triazolyl, imidazolyl, pyrazolyl, isoxazolyl, isothiazolyl, pyridyl, piperidinyl and pyrazinyl.

Particular examples of non-nitrogen containing heteroaryl include pyranyl, 2-furyl, 3-furyl, 2-thienyl, 3-thienyl, benzofuryl, benzothienyl, and the like.

Particular examples of partially saturated and saturated heterocyclyl include pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a, 9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl, and the like.

“Heterocycle” means a ring comprising at least one carbon atom and at least one other atom selected from N, O and S. Examples of heterocycles that may be found in the claims include, but are not limited to, the following:

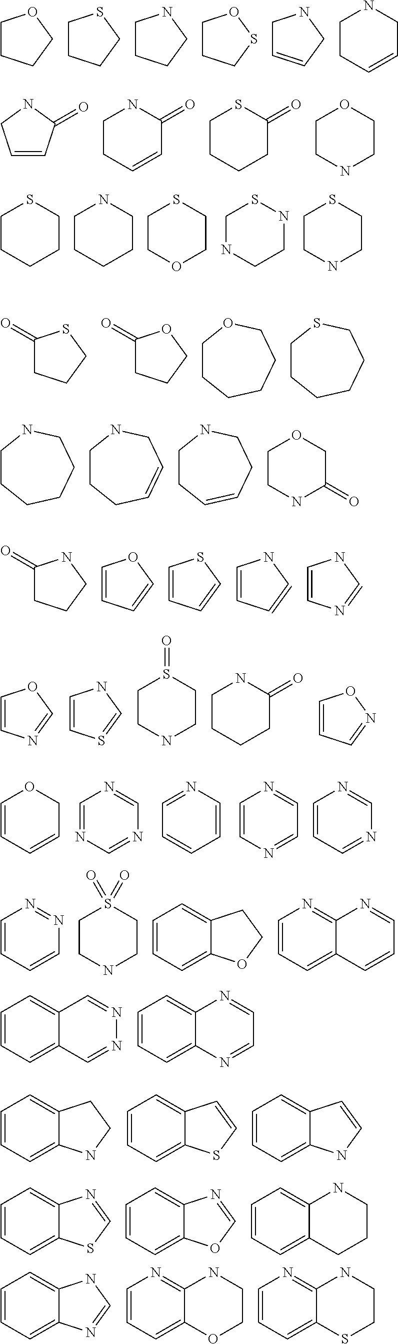

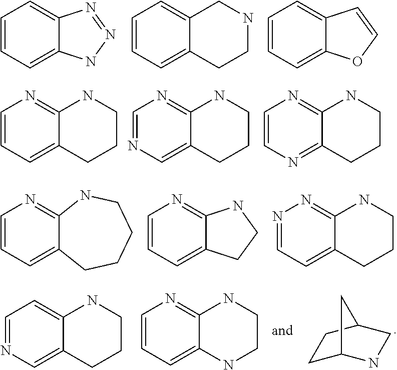

The terms “aralkyl” or “arylalkyl” embraces aryl-substituted alkyl radicals. Preferable aralkyl radicals are “lower aralkyl” radicals having aryl radicals attached to alkyl radicals having one to six carbon atoms. Even more preferred are “phenylalkylenyl” attached to alkyl portions having one to three carbon atoms. Examples of such radicals include benzyl, diphenylmethyl and phenylethyl. The aryl in said aralkyl may be additionally substituted with halo, alkyl, alkoxy, halkoalkyl and haloalkoxy. The term “optionally substituted phenylalkylenyl” when used in a linker may be divalent on either the alkyl portion or the phenyl ring and the alkyl portion.

The term “halo” means halogens such as fluorine, chlorine, bromine or iodine atoms.

The term “haloalkyl” embraces radicals wherein any one or more of the alkyl carbon atoms is substituted with halo as defined above. Specifically embraced are monohaloalkyl, dihaloalkyl and polyhaloalkyl radicals including perhaloalkyl. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Even more preferred are lower haloalkyl radicals having one to three carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perfluoroalkyl” means alkyl radicals having all hydrogen atoms replaced with fluoro atoms. Examples include trifluoromethyl and pentafluoroethyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl. Even more preferred are lower hydroxyalkyl radicals having one to three carbon atoms.

The term “alkoxy” embrace linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. Even more preferred are lower alkoxy radicals having one to three carbon atoms. Alkoxy radicals may be further substituted with one or more halo atoms, such as fluoro, chloro or bromo, to provide “haloalkoxy” radicals. Even more preferred are lower haloalkoxy radicals having one to three carbon atoms. Examples of such radicals include fluoromethoxy, chloromethoxy, trifluoromethoxy, trifluoroethoxy, fluoroethoxy and fluoropropoxy.

The terms “polymorph” and “polymorphic form” refer to different crystalline forms of a single compound. That is, polymorphs are distinct solids sharing the same molecular formula, yet each polymorph may have distinct solid state physical properties. Therefore, a single compound may give rise to a variety of polymorphic forms where each form has different and distinct solid state physical properties, such as different solubility profiles, dissolution rates, melting point temperatures, flowability, and/or different X-ray diffraction peaks. The differences in physical properties may affect pharmaceutical parameters such as storage stability, compressibility and density (which can be important in formulation and product manufacturing), and dissolution rate (which can be an important factor in bioavailability). Techniques for characterizing polymorphic forms include, but are not limited to, X-ray powder diffractometry (XRPD), differential scanning calorimetry (DSC), thermal gravimetric analysis (TGA), single-crystal X-ray diffractometry (XRD), vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility measurements, dissolution measurements, elemental analysis and Karl Fischer analysis.

The term “about” preceding one or more peak positions in an X-ray powder diffraction pattern means that all of the peaks of the group which it precedes are reported in terms of angular positions (two theta) with an allowable variability of ±0.3°. The variability of ±0.3° is intended to be used when comparing two powder X-ray diffraction patterns. In practice, if a diffraction pattern peak from one pattern is assigned a range of angular positions (two theta) which is the measured peak position ±0.3° and if those ranges of peak positions overlap, then the two peaks are considered to have the same angular position. For example, if a peak from one pattern is determined to have a position of 11.0°, for comparison purposes the allowable variability allows the peak to be assigned a position in the range of 10.7°-11.3°.

The term “amorphous” means a solid in a solid state that is a non-crystalline state. Amorphous solids are disordered arrangements of molecules and therefore possess no distinguishable crystal lattice or unit cell and consequently have no definable long range ordering. The solid state form of a solid may be determined by polarized light microscopy, X-ray powder diffraction (“XRPD”), differential scanning calorimetry (“DSC”), or other standard techniques known to those of skill in the art.

AMG151 is (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol.

The abbreviations in the specification correspond to units of measure, techniques, properties, or compounds as follows:

| | |
|---|---|
| Anh. | Anhydrous |
| BHT | butylhydroxytoluene |
| CBZ | Benzyloxycarbonyl |
| CH₂Cl₂, DCM | dichloromethane, methylene chloride |
| DIPEA | di-isopropylethylamine |
| DMAC | N,N-dimethylacetamide |
| DMF | dimethylformamide |
| DMSO | dimethylsulfoxide |
| EtOAc | ethyl acetate |
| EtOH | ethanol |
| h | hour(s) |
| HCl | hydrochloric acid |
| H₂O | water |
| H₂O₂ | hydrogen peroxide |
| HMDS | hexamethyldisilazane |
| HSO₃⁻ | bisulfate |
| IPA | isopropyl alcohol |
| IPAC | isopropyl acetate |
| Kg | kilogram |
| KOAc | potassium acetate. |
| KOtBu | potassium tert-butoxide |
| K₂S₂O₅ | potassium metabisulfite |
| K₂CO₃ | potassium carbonate |
| L | liter |
| L jacketed reactor | Liter jacketed reactor |
| MeTHF | 2-methyl tetrahydrofuran |
| M | molar |
| mCPBA | metachloroperbenzoic acid |
| MeCN | acetonitrile |
| MEK: | methyl ethyl ketone |
| MeOH | methanol |
| MgSO₄ | magnesium sulfate |
| Min | minutes |
| mL | milliliter(s) |
| mM | millimolar |
| mmole | millimole(s) |
| MMPP | Magnesium monoperoxyphthalate hexahydrate |
| MoO₂Cl₂ | Molybdenum dichloride dioxide |
| MoO₂(acac)₂ | Molybdenyl(VI) acetylacetonate |
| MSA | methanesulfonic acid |
| MsCl | mesyl chloride, methylsulfonyl chloride |
| MTBE | methyl tertbutyl ether |
| N₂ | nitrogen |
| NCS | N-chlorosuccinimide |
| NMO | N-Methylmorpholine-N-oxide |
| NMP | N-methyl pyrrolidone |
| NH₂OH | hydroxylamine |
| NH₂OH HCl | hydroxylamine hydrochloride |
| (NH₄)₆Mo₇O₂₄ | Ammonium molybdate |
| NaSCN | sodium thiocyanate |
| NaHCO₃ | sodium bicarbonate |
| Na₂CO₃ | sodium carbonate |
| NaIO₄ | sodium iodate |
| NaHSO₃ | sodium bisulfite |
| NaOAc | sodium acetate |
| Na₂SO₃ | sodium sulfite |
| Na₂S₂O₅ | sodium metabisulfite |
| NaWO₄ | sodium tungstate |
| Pb(OAc)₄ | Lead tetraacetate |
| RT | room temperature |
| Sat. | saturated |
| TBHP | tert-Butyl hydroperoxide |
| THF | tetrahydrofur\dot{a}n |
| TMSCl | Chlorotrimethylsilane |
| TPAP | tetrapropylammonium perruthenate |
| UHP | urea-H₂O₂ |
| μL | microliter(s) |
| XRPD | X-ray Powder Diffraction |
| DSC | Differential scanning calorimetry |
| TGA | Thermogravimetric analysis |
| FeSIF | Feed Simulated Intestinal Fluid |
| FaSIF | Fasted Simulated Intestinal Fluid |
| SGF | Simulated Gastric Fluid |

It is believed the chemical formulas and names used herein correctly and accurately reflect the underlying chemical compounds. However, the nature and value of the present invention does not depend upon the theoretical correctness of these formulas, in whole or in part. Thus it is understood that the formula used herein, as well as the chemical names attributed to the correspondingly indicated compounds, are not intended to limit the invention in any way, including restricting it to any specific tautomeric form or to any specific optical or geometric isomer.

The previous definitions are provided for the full understanding of terms and abbreviations used in this specification.

### General Procedures

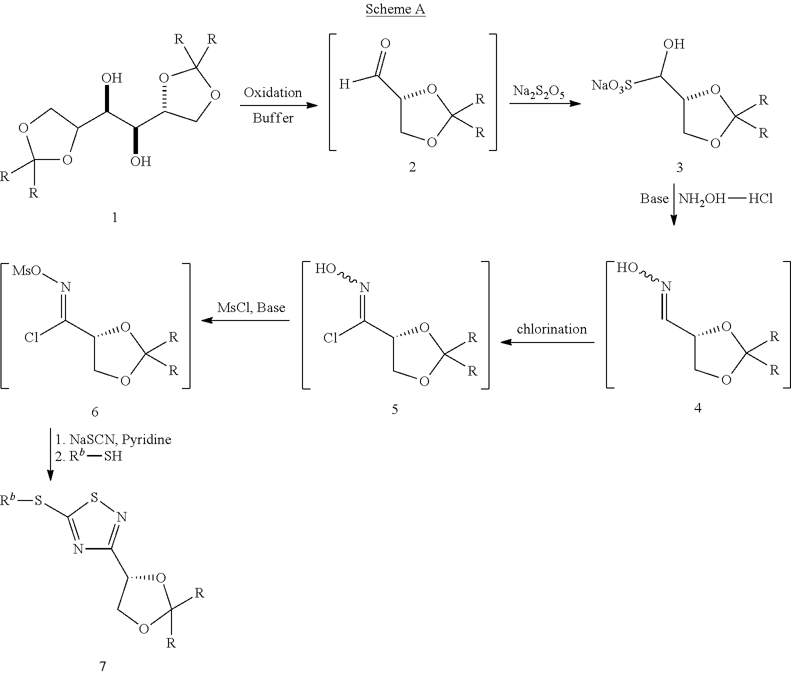

The present invention, as shown in Scheme A, involves formation of protected glyceraldehydes 3 as well as formation of the thiadiazole sulfides 7. The invention also relates to compounds where R is C1-3 alkyl, or the two R groups together form cyclohexyl and where R^{b} is aryl, alkyl, aralkyl, or 5-6 membered heterocyclyl. In another embodiment of the invention, R^{b} is C6-10 membered aryl, C1-6 alkyl, C6-10 aryl-C1-3 alkyl, 5-6 membered heteroaryl or 5-6 membered saturated or partially saturated heterocyclyl. In another embodiment of the invention, the aryl, alkyl, arylalkyl, or 5-6 membered heterocyclyl substituents are optionally substituted with one or more substituents selected from lower alkyl, halo, haloalkyl, and the like.

Embodiments of the process include oxidative cleavage of a substituted diol 1 to give aldehyde 2. Embodiments of the process include oxidizing cleavage agent such as NaIO₄, chromic acid, or Pb(OAc)₄. In certain embodiments of the invention, NaIO₄ is used for the oxidative cleavage. Embodiments of the process include NaIO₄ in an amount of at least about 1 equivalent per mole of the diol employed. The invention also relates to the use of about 1.4-1.5 equivalents of NaIO₄.

Embodiments of the process include the oxidizing cleavage in an organic solvent such as CH₂Cl₂ or EtOAc.

Embodiments of the process include an aqueous buffer such as NaHCO₃, at about 0.3 eq., in the presence of H₂O. Preferably the pH of the oxidative cleavage is maintained higher than 0.8, preferably above 3.

Treatment of an alcoholic aldehyde solution with a salt of SO₃⁻², such as Na₂S₂O₅, NaHSO₃, or Na₂SO₃, at a temperature above RT, preferably at above 35° C., more preferably at a temperature of about 50° C., provides a diastereomeric mixture of the bisulfite adduct 3. Embodiments of the process include treatment with Na₂S₂O₅ in an amount of about 0.5-2 equivalents per mole of the aldehyde employed. The invention also relates to the use of about 0.5 equivalents of Na₂S₂O₅.

Treatment of the adduct 3 in an organic solvent, such as MeTHF, with an aqueous solution of NH₂OH—HCl and a base such as K₂CO₃ or Na₂CO₃, gives the oxime 4. To the anhydrous oxime 4 in a solvent such as a mixture of DMAC/MeTHF, is added a catalytic amount of HCl in a solvent such as dioxane followed by halogenation, such as with Cl₂ or NCS, to give the corresponding chloro-oxime 5 intermediate. The chloro-oxime 5 is reacted with MsCl in the presence of base, such as DIPEA, to give the chloro-mesylate 6, at a temperature below RT, preferably at a temperature of about 0° C. Reaction of chloro-mesylate 6 with NaSCN, in a solvent such as MeTHF, at a temperature of about RT, and in the presence of organic base, such as pyridine, gives the acyl thioisocyanate intermediate. Treatment of the acyl thioisocyanate with a substituted thiol, such as toluene sulfide, in the presence of 1-2 equivalents of organic base, such as pyridine, in a non-polar solvent such as MeTHF, at a temperature below RT, preferably at about 0° C., gives 1,2,4-thiadiazole 7.

The invention also relates to a process in an atmosphere where minimal oxygen is present, such as in a N₂ environment.

The present invention also relates to a process where the bisulfite adduct 3 is isolated prior to the cyclization step. Alternatively, the bisulfite adduct 3 is not isolated prior to formation of the thiadiazole.

Embodiments of the process include reaction in a non-aqueous solvent environment. Such solvents include MeTHF, MTBE, IPAC, heptane, hexane, toluene, benzene, xylenes, IPA, dioxane, CH₂Cl₂, EtOH, MeCN and THF. The present invention also relates to a process where a mixture of solvents is utilized. In certain embodiments of the invention, MeTHF is used as the solvent. Where the term “non-aqueous” is used, it is not to intend that water is not generated by a reaction step.

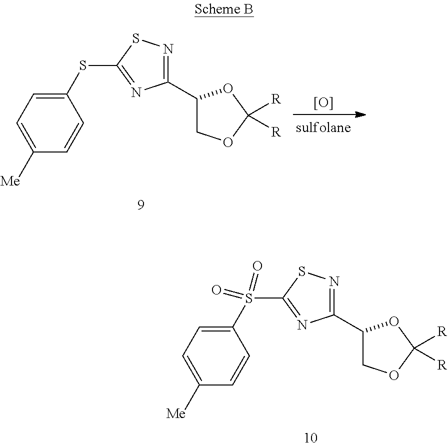

The present invention, as shown in Scheme B, describes oxidation of 3-[(4S)-2,2-dimethyl-1,3-dioxolan-4-yl]-5-[(4-methylphenyl)sulfanyl]-1,2,4-thiadiazole 9. For example, 3-[(4S)-2,2-dimethyl-1,3-dioxolan-4-yl]-5-[(4-methylphenyl)sulfanyl]-1,2,4-thiadiazole is treated with an oxidizing agent, to provide the corresponding sulfones 10 [where R is methyl].

Embodiments of the process include an oxidizing agent selected from peroxide related agents such as urea-H₂O₂, aqueous H₂O₂, and peracid based reagents such as mCPBA; peracetic acid or MMPP. The invention also relates to the use of urea-H₂O₂.

Embodiments of the process include a peroxide related oxidizing agent in the presence of a catalyst, for example (NH₄)₆Mo₇O₂₄. Embodiments of the process include a peroxide related oxidizing agent in the presence of a catalyst, where less than about 10 wt % of catalyst is used. Embodiments of the process include a peroxide related oxidizing agent in the presence of a catalyst, where less than about 5 wt % of catalyst is used.

Embodiments of the process include oxidation in the presence of a solvent such as acetonitrile or sulfolane.

Embodiments of the process include an oxidation carried out at a temperature of above about −15° C. and the temperature of reflux of the solution. Embodiments of the process include an oxidation carried out at a temperature of above about −15° C. and about 50° C. The invention also relates to an oxidation carried out at a temperature of above about RT.

Embodiments of the process include oxidizing agent in an amount of more than about 1 equivalent per mole of the sulfide employed. The invention also relates to the use of about 2.5 equivalents of oxidizing agent.

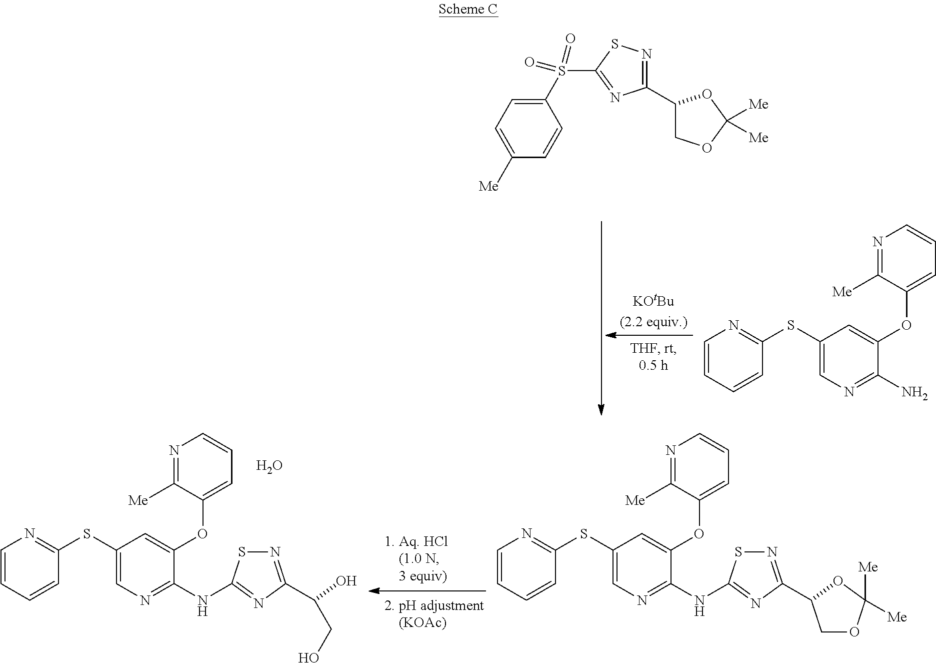

The present invention, as shown in Scheme C, involves a process for the formation of the hydrate form of AMG151 comprising treating 3-((2-methylpyridin-3-yl)oxy)-5-(pyridin-2-ylthio)pyridin-2-amine with a base and (S)-3-(2,2-dimethyl-1,3-dioxolan-4-yl)-5-tosyl-1,2,4-thiadiazole to form the protected diol, (S)-3-(2,2-dimethyl-1,3-dioxolan-4-yl)-N-(3-((2-methylpyridin-3-yl)oxy)-5-(pyridin-2-ylthio)pyridin-2-yl)-1,2,4-thiadiazol-5-amine. The process can be performed in a solvent, such as THF, at a temperature of about −20° C. to about RT. Examples of base used to deprotonate, includes potassium tert-butoxide. Preferably an excess of base is used. Preferably, the base is added to the pyridine-2-amine prior to addition of the sulfone

Deprotection of the protected diol, (S)-3-(2,2-dimethyl-1,3-dioxolan-4-yl)-N-(3-((2-methylpyridin-3-yl)oxy)-5-(pyridin-2-ylthio)pyridin-2-yl)-1,2,4-thiadiazol-5-amine with acid forms the diol. Examples of acids include HCl, sulfuric acid, methanesulfonic acids, e.g. aqueous HCl, such as 1N HCl. Adjustment of pH, such as with base, e.g. KOAc, phosphates, carbonates, or alkylamines, allows one to isolate the desired compound (the hydrate of AMG151) as a crystalline material. Preferably, the deprotection is achieved in an excess of acid, e.g. 3 equivalents of aqueous HCl (1.0 N). Preferably, the deprotection is achieved at about ambient temperature.

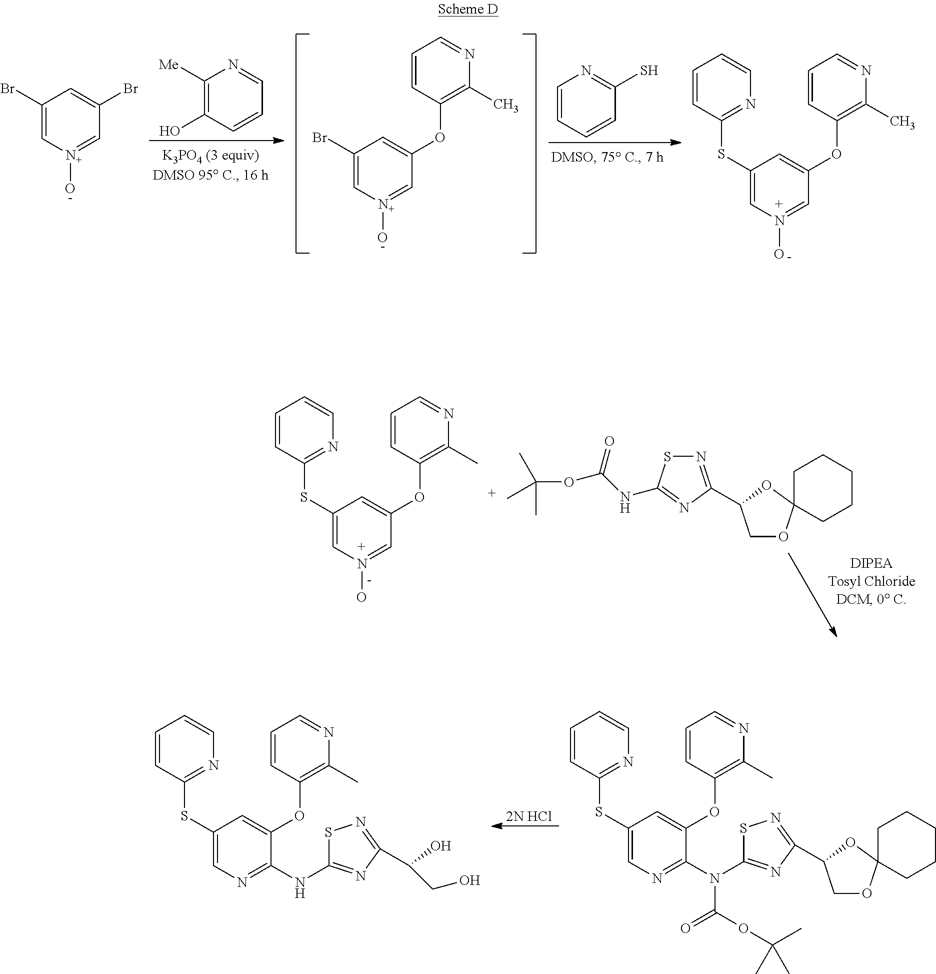

Scheme D describes a direct amination route to AMG151 via a pyridine N-oxide. 3-((2-Methylpyridin-3-yl)oxy)-5-(pyridin-2-ylthio)pyridine 1-oxide is combined with a protected 2-aminothiadiazole

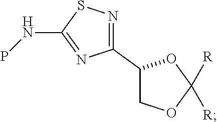

where R is aryl, alkyl or the two R groups together form cycloalkyl, and wherein P is a protecting group, preferably P is Boc, in a solvent, such as DCM. Coupling of the pyridine oxide with a protected amino-thiadiazole, such as in the presence of a base and TsCl in an appropriate solvent yields the protected pyridine-2-aminothiadiazole. An example of a base is DIPEA. The process can be performed in a solvent, such as THF or DCM, at a temperature of about 0° C. to about RT. Deprotection, such as with treatment with acid, provides the desired compound. Examples of the acid include HCl, e.g. aqueous HCl, such as 2N HCl. The deprotection can be performed at a temperature over RT, preferably above about 50° C. and more preferably at about 65° C.

The coupling reaction described could utilize alternative protecting groups for the diol function other than the cyclohexyl acetal shown above.

In general, any amino protecting group of the thiadiazole which increases the acidity of the NH proton and can be conveniently cleaved after the coupling reaction would be amenable to the approach described here. Other examples include but are not limited to benzyl, CBZ, and t-butyl. Additionally, other amino-thiadiazole nucleophiles could likely perform well in this coupling such as structures shown below:

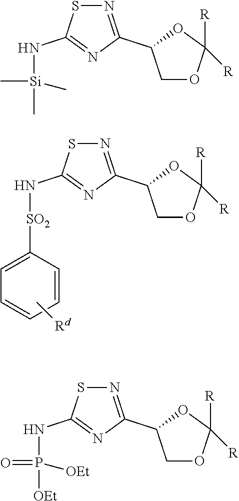

Alternative activators for the coupling reaction describe here may include any reagent known in the chemical literature to activate pyridine N-oxides towards nucleophilic attack by N, C, and O based nucleophiles. Examples include POCl₃, sulfonyl chlorides, sulfonyl anhydrides, oxalyl chloride, acetic anhydride and triflic anhydride.

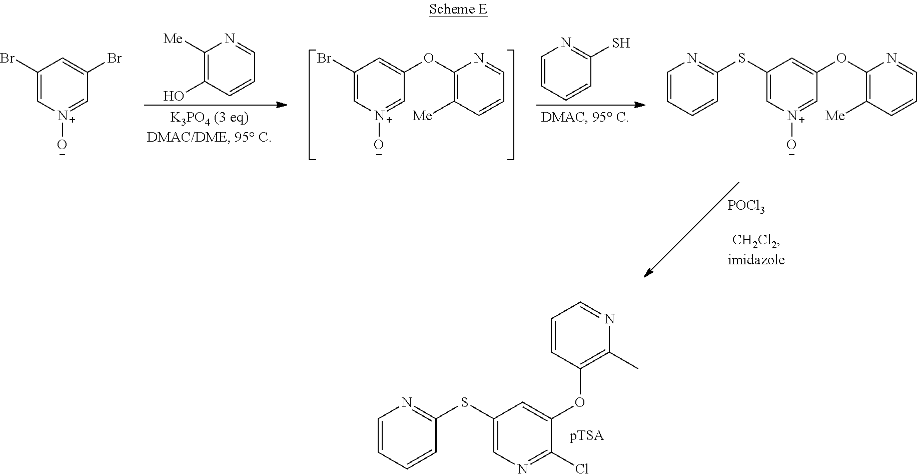

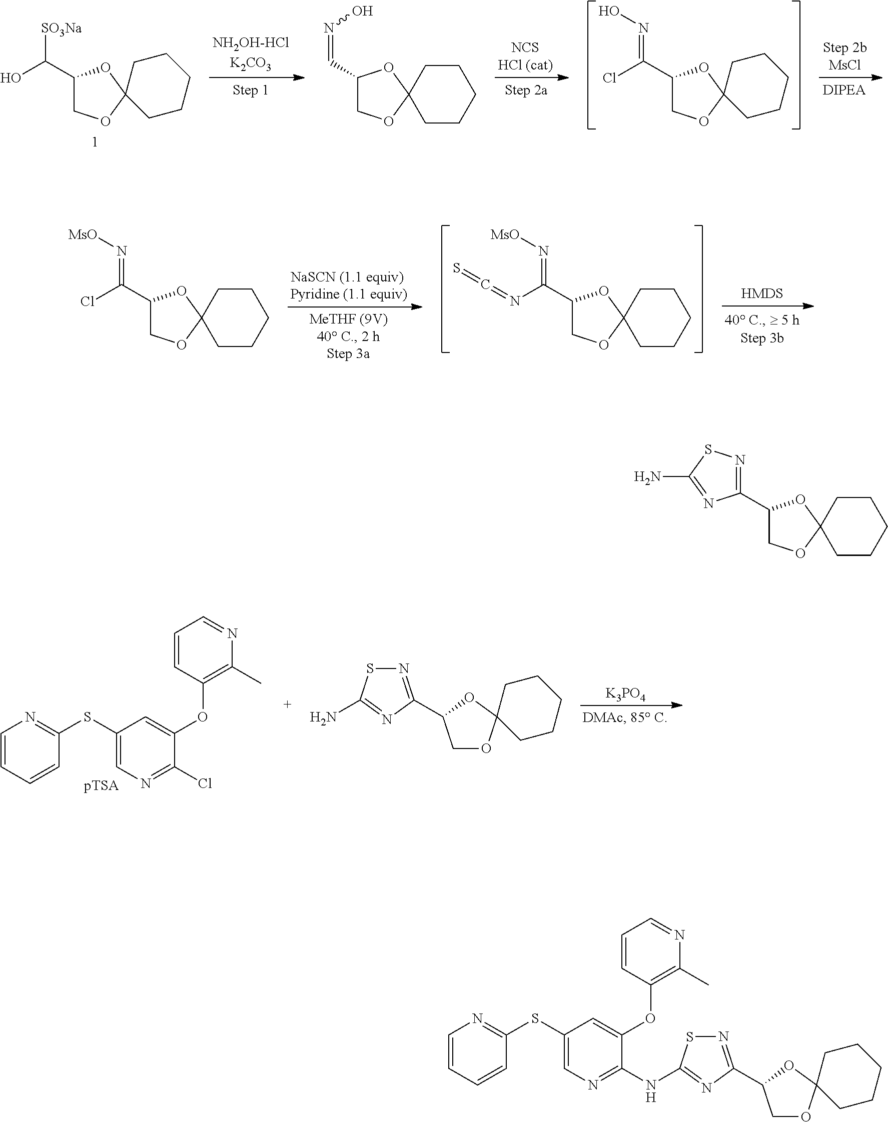

The present invention, as shown in Scheme E, involves a process for the formation of AMG151 comprising coupling 2-chloro-3-((2-methylpyridin-3-yl)oxy)-5-(pyridin-2-ylthio)pyridine and (S)-3-(1,4-dioxaspiro[4.5]decan-2-yl)-1,2,4-thiadiazol-5-amine in the presence of base such as carbonates or K₃PO₄ and a solvent such as DMAC, DMSO, NMP and the like. Subsequent treatment with acid, such as HCl, as shown in Scheme C, yields AMG151.

The 2-chloro-3-((2-methylpyridin-3-yl)oxy)-5-(pyridin-2-ylthio)pyridine is prepared by coupling of 3,5-dibromopyridine N-oxide with 2-methylpyridin-3-ol in the presence of base such as carbonates or K₃PO₄ and a solvent such as DMAC, DMSO, NMP and the like, followed by coupling with 2-mercaptopyridine and a solvent such as DMAC. The couplings can be performed at a temperature over RT, preferably above about 75° C. and more preferably at about 95° C. Subsequent halogenation, such as with POCl₃, provides the desired 2-chloropyridine. The halogenation can be performed at a temperature below RT, preferably at about 10° C.

The (S)-3-(1,4-dioxaspiro[4.5]decan-2-yl)-1,2,4-thiadiazol-5-amine is prepared reacting the bisulfite adduct with hydroxyamine to give oxime intermediate, which was chlorinated with NCS in the presence of catalytic amount of HCl to form chloro-oxime. Mesylation of the chloro-oxime gives the cyclohexyl chloromesylate which is converted to acylthiocyanate by the addition of NaSCN, pyridine, and MeTHF. Cyclization is accomplished with an ammonia surrogate (HMDS) which is activated with catalytic water in the batch to form the 2-amino-thiadiazole.

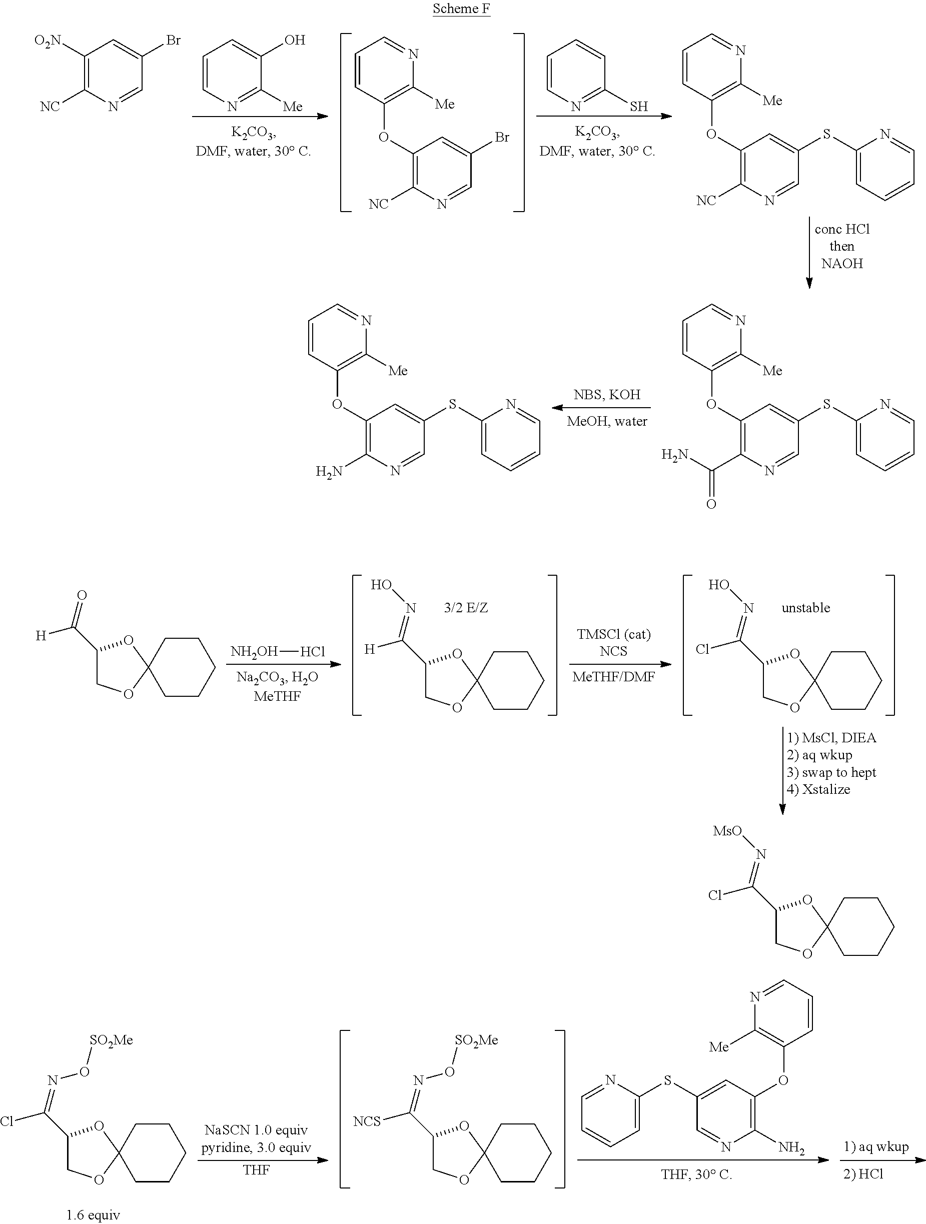

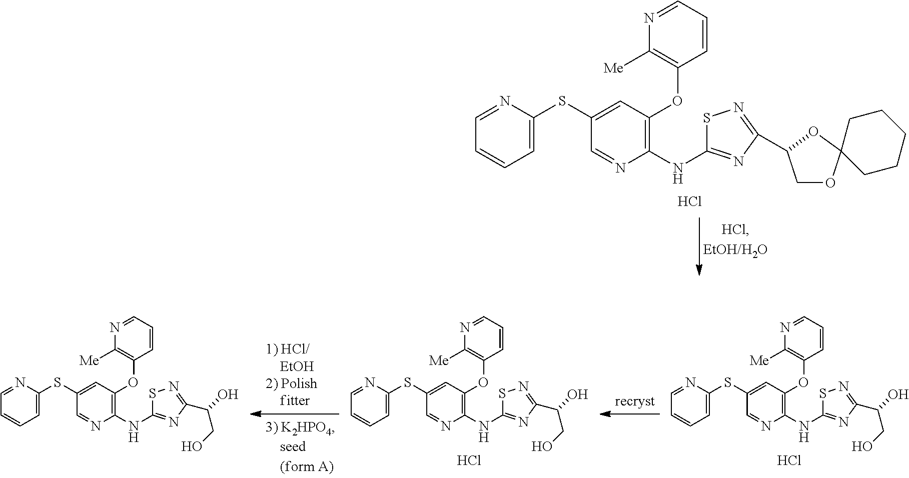

The present invention, as shown in Scheme F, involves a process for the formation of AMG151 comprising coupling 2-amino-3-((2-methylpyridin-3-yl)oxy)-5-(pyridin-2-ylthio)pyridine and (R, E)-N-((methylsulfonyl)oxy)-1,4-dioxaspiro[4.5]decane-2-carbimidoyl chloride in the presence of sodium isothiocyanate and pyridine. Subsequent treatment with acid, such as HCl, yields AMG151.

The 2-amino-3-((2-methylpyridin-3-yl)oxy)-5-(pyridin-2-ylthio)pyridine is prepared from sequentially coupling 5-bromo-3-nitropicolinonitrile and 2-methylpyridin-3-ol then pyridin-2-thiol, in the presence of base, e.g. K₂CO₃, to yield the 3-((2-methylpyridin-3-yl)oxy)-5-(pyridin-2-ylthio) picolinonitrile. The couplings can be performed at a temperature about RT, preferably at about 30° C. Hydrolysis of the nitrile to the amide through, followed by treatment bromination and base, yields the amine.

The (R, E)-N-((methylsulfonyl)oxy)-1,4-dioxaspiro[4.5]decane-2-carbimidoyl chloride is prepared from (R)-1,4-dioxaspiro[4.5]decane-2-carbaldehyde via sequential treatment with hydroxylamine, TMSCl and NCS, and MsCl.

Also provided is a method of treating a medical condition or disorder in a subject where treatment with a glucokinase activator is indicated, comprising administering, for example orally, a composition of the invention in a therapeutically effective amount. Such method is particularly useful where the medical condition or disorder is diabetes.

The invention provides novel hydrate forms of AMG151, preferably the monohydrate form. In addition to monohydrate form of AMG151 per se, the invention provides AMG151 drug substance that comprises the monohydrate form of AMG151. At least a detectable amount of the monohydrate form of AMG151 is present. Preferably, about 10% to about 100%, more preferably about 25% to about 100%, still more preferably about 60% to about 100%, and even more preferably about 80% to about 100%, by weight of the AMG151 in an AMG151 drug substance of the invention is the hydrate form of AMG151, preferably the monohydrate form. In a particular embodiment, substantially all of the AMG151 is hydrate form, i.e., the AMG151 drug substance is substantially pure monohydrate form of AMG151.

The invention provides novel forms of free base AMG151, specifically the Form A or Form F. In addition to Forms A and F of AMG151 free base per se, the invention provides AMG151 drug substance that comprises Forms A and F of AMG151 free base. At least a detectable amount of either Form A or F of AMG151 free base of AMG151 is present. In one embodiment, 100% by weight of the AMG151 in an AMG151 drug substance of the invention is Form A. In one embodiment, about 0.1% by weight of the AMG151 in an AMG151 drug substance of the invention is Form A and 99.9% is Form F. In one embodiment, about 10% by weight of the AMG151 in an AMG151 drug substance of the invention is Form A and 90% is Form F. In one embodiment, about 20% by weight of the AMG151 in an AMG151 drug substance of the invention is Form A and 80% is Form F. In one embodiment, about 25% by weight of the AMG151 in an AMG151 drug substance of the invention is Form A and 75% is Form F. In one embodiment, about 30% by weight of the AMG151 in an AMG151 drug substance of the invention is Form A and 70% is Form F. In one embodiment, about 40% by weight of the AMG151 in an AMG151 drug substance of the invention is Form A and 60% is Form F. In one embodiment, about 50% by weight of the AMG151 in an AMG151 drug substance of the invention is Form A and 50% is Form F. In one embodiment, about 60% by weight of the AMG151 in an AMG151 drug substance of the invention is Form A and 40% is Form F. In one embodiment, about 75% by weight of the AMG151 in an AMG151 drug substance of the invention is Form A and 25% is Form F. In one embodiment, about 80% by weight of the AMG151 in an AMG151 drug substance of the invention is Form A and 20% is Form F. In one embodiment, about 90% by weight of the AMG151 in an AMG151 drug substance of the invention is Form A and 10% is Form F. In one embodiment, about 99.9% by weight of the AMG151 in an AMG151 drug substance of the invention is Form A and 0.1% is Form F. In one embodiment, 100% by weight of the AMG151 in an AMG151 drug substance of the invention is Form F.

Usually, about 10% to about 100%, more often about 25% to about 100%, still more often about 60% to about 100%, and even more often about 80% to about 100%, by weight of the AMG151 in an AMG151 drug substance of the invention is either Form A or F of AMG151 free base. In a particular embodiment, substantially all of the AMG151 is either Form A or Form F of AMG151 free base, i.e., the AMG151 drug substance is substantially AMG151 Form A or substantially AMG151 Form F.

In one embodiment, provided herein is crystalline polymorph of AMG151 freebase, substantially in the form of Form A. As used herein, the term “substantially in the form of Form A” means the polymorphic form includes less than about 15% by weight of other forms, including other polymorphic forms and amorphous forms. In certain embodiments, the substantially pure polymorphic form includes less than about 10% by weight of other forms, including other polymorphic forms and amorphous forms. In certain embodiments, the substantially pure polymorphic form includes less than about 5% by weight of other forms, including other polymorphic forms and amorphous forms. In certain embodiments, the substantially pure polymorphic form includes less than about 1% by weight of other forms, including other polymorphic forms and amorphous forms.

Also provided herein is a process for preparing the crystalline polymorph of AMG151 freebase, substantially in the form of Form A, comprising: combining AMG151 hydrochloride with water, ethyl alcohol and 37% aqueous hydrochloric acid to provide AMG151 hydrate, Form C; adding K₂HPO₄, water and ethyl alcohol to the acidic solution to adjust the pH; adding ethanol to the solution containing AMG151 hydrate, Form C until the ratio of water:ethanol is 60:40; and stirring the solution at about 50° C. to provide AMG151 freebase, substantially in the form of Form A. Form A may be isolated by standard methods.

Also provided herein is a process for preparing the crystalline polymorph of AMG151 hydrochloride, substantially in the form of Form C, comprising: combining AMG151 hydrochloride with water, ethyl alcohol and 37% aqueous hydrochloric acid; and adding K₂HPO₄, water and ethyl alcohol to the acidic solution to provide the crystalline polymorph of AMG151 hydrate, substantially in the form of Form C. Form C may be isolated by standard methods. Crystalline polymorph of AMG151 hydrate, substantially in the form of Form C is useful for preparing AMG151 freebase, substantially in the form of Form A as described herein.

The invention provides novel solvate forms of AMG151, preferably the fumarate solvate form. In addition to fumarate solvate form of AMG151 per se, the invention provides AMG151 drug substance that comprises the fumarate solvate form of AMG151. At least a detectable amount of the fumarate solvate form of AMG151 is present. Preferably, about 10% to about 100%, more preferably about 25% to about 100%, still more preferably about 60% to about 100%, and even more preferably about 80% to about 100%, by weight of the AMG151 in an AMG151 drug substance of the invention is the fumarate solvate salt form. In a particular embodiment, substantially all of the AMG151 is fumarate solvate form, i.e., the AMG151 drug substance is substantially pure fumarate solvate form of AMG151.

In one embodiment, the amount of fumarate solvate form of AMG151 in an AMG151 drug substance is sufficient to treat diabetes, wherein all, or a substantial portion of, the AMG151 is substantially fumarate solvate form.

In one embodiment, the amount of either Form A or Form F of AMG151 free base in an AMG151 drug substance is sufficient to treat diabetes, wherein all, or a substantial portion of, the AMG151 is substantially either Form A or Form F of AMG151 free base.

In one embodiment, the amount of monohydrate form of AMG151 in an AMG151 drug substance is sufficient to treat diabetes, wherein all, or a substantial portion of, the AMG151 is substantially monohydrate form of AMG151.

The hydrate form of AMG151 or AMG151 drug substance of the invention can be prepared by any suitable process, not limited to processes described herein. AMG151 can be added to an aqueous solution, including alcoholic solutions, e.g. solutions with ethanol. For example solutions can be used with less than about 25% ethanol. Preferably the AMG151 is dissolved in the aqueous solution. The aqueous solution is preferably heated to a temperature above room temperature, such as above about 50° C., preferably at boiling. Hydrate material in a solid form can be collected upon cooling. Preferably the cooling occurs over three hours.

Alternatively, the crystalline polymorphic form of AMG151 monohydrate, Form C, or AMG151 hydrate, Form C drug substance of the invention can be prepared by the general process described below, such as in Examples 2 and 3, and Examples 22 and 23. AMG151 can be added to an aqueous acidic solution. In one embodiment, the aqueous acidic solution is at a pH below 2. The aqueous solution is maintained at a temperature at about 25±5° C. Modification of the pH, such as treatment with base, for example an aqueous base, such as aqueous KOAc, results in crystallization of the AMG151 hydrate. In one embodiment the pH is adjusted with a base to maintain the pH at above 3. The crystalline polymorphic form of AMG151 monohydrate, Form C or AMG151 monohydrate, Form C drug substance in a solid form can be isolated by standard methods.

Also provided herein is a process for the preparation of the crystalline polymorph of AMG151 monohydrate, Form C, with consistent particle size distribution. It was discovered that control of particle size distribution of AMG151 monohydrate, Form C could be obtained by identifying the seed point by pH and growing large particles by controlled crystallization. The target particle size distribution could be obtained by pin milling. However, there is a risk of dehydration of the monohydrated Form C during the milling step.

Accordingly, also provided herein is a process of preparation of the crystalline polymorph of AMG151 monohydrate, Form C, wherein consistent particle size distribution is achieved without milling. It was unexpectedly discovered that maintaining a pH of 1.73-2.15 at seed point resulted in robust and reproducible crystallization of AMG151 monohydrate, Form C with more control over particle size distribution. The use of an appropriate buffer system was discovered to widen the seeding window with respect to pH and improved the reproducibility of the particle size distribution across multiple reaction scales. It was further unexpectedly discovered that use of sulfuric acid rather than other acids (such as hydrochloric acid) resulted in a more consistent particle size distribution. It was further unexpectedly discovered that the particle size of the seed and the seed load have a strong influence on the particle size distribution. It was observed that seeding with pin-milled AMG151 monohydrate, Form C, and a load of the pin-milled seed of about 2-3 weight percent resulted in more consistent particle size distribution.

In one embodiment, provided herein is a process for preparing AMG151 monohydrate, Form C with consistent particle size distribution comprising: combining AMG151 with a 1N aqueous sulfuric acid solution at ambient temperature; adding water to said acidic solution; adding a 1.0 N aqueous potassium acetate solution to said acidic solution at ambient temperature to adjust the pH of the acidic solution to between 1.7 and 2.1; seeding said acidic solution with AMG151 monohydrate, Form C when the pH of the acidic solution is between 1.7 and 2.1; and allowing said Form C to crystallize from said solution. In one embodiment, about 2-3 weight percent of the seed is added. In one embodiment, the seed is pin-milled AMG151 monohydrate, Form C. In one embodiment, the particle size of pin milled material is d₉₀≦5-50 μm. In one embodiment, the particle size of pin-milled seed material is d₉₀≦100 μm. In one embodiment, said process provides AMG151 monohydrate, Form C having a particle size d₉₀ less than 20 μm. In one embodiment, said process provides AMG151 monohydrate, Form C having a particle size d₉₀ less than 50 μm. In one embodiment, said process provides AMG151 monohydrate, Form C having a particle size d₉₀ less than 100 μm. In one embodiment, said process provides AMG151 monohydrate, Form C having a particle size d₉₀ less than 200 μm. In one embodiment, said process provides AMG151 monohydrate, Form C having a particle size distribution profile of: d₁₀ between about 3-6 μm, d₅₀ between about 15 and 21 μm, and d₉₀ between about 42 and 61 μm.

Administration and Pharmaceutical Compositions

In general, the forms of this invention can be administered in a therapeutically effective amount by any of the accepted modes of administration for agents that serve similar utilities. The actual amount of a compound of this invention, i.e., the active ingredient, depends upon numerous factors, such as the severity of the disease to be treated, the age and relative health of the subject, the potency of the compound used, the route and form of administration, and other factors.

Therapeutically effective amounts of AMG151 may range from approximately 0.1-1000 mg per day.

In general, forms of this invention can be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

AMG151 drug substance or drug powder prepared according to the above process or any other process can be administered orally, rectally or parenterally without further formulation, or in simple suspension in water or another pharmaceutically acceptable liquid. Alternatively, the AMG151 drug substance or drug powder can be directly filled into capsules for oral administration. Preferably, however, AMG151 drug substance or drug powder is subjected to further processing, typically with one or more excipients, to prepare a pharmaceutical composition, for example an oral dosage form, as described herein below.

The forms of AMG151 or AMG151 drug substance as provided herein can be further formulated together with one or more pharmaceutically acceptable excipients to produce a pharmaceutical composition. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients include, by way of illustration and not limitation, diluents, disintegrants, binding agents, adhesives, wetting agents, lubricants, glidants, crystallization inhibitors, surface-modifying agents, substances added to mask or counteract a disagreeable taste or odor, flavors, dyes, fragrances, and substances added to improve appearance of the composition.

Excipients employed in compositions of the invention can be solids, semi-solids, liquids or combinations thereof. Compositions of the invention containing excipients can be prepared by any known technique of pharmacy that comprises admixing an excipient with a drug or therapeutic agent. A composition of the invention contains a desired amount of AMG151 crystalline form per dose unit and, if intended for oral administration, can be in the form, for example, of a tablet, a caplet, a pill, a hard or soft capsule, a lozenge, a cachet, a dispensable powder, granules, a suspension, an elixir, a liquid, or any other form reasonably adapted for such administration. If intended for parenteral administration, it can be in the form, for example, of a suspension. If intended for rectal administration, it can be in the form, for example, of a suppository. Presently preferred are oral dosage forms that are discrete dose units each containing a predetermined amount of the drug, such as tablets or capsules.

Non-limiting examples follow of excipients that can be used to prepare pharmaceutical compositions of the invention. Compositions of the invention optionally comprise one or more pharmaceutically acceptable diluents as excipients. Suitable diluents illustratively include, either individually or in combination, lactose, including anhydrous lactose and lactose monohydrate; starches, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, food grade sources of α- and amorphous cellulose (e.g., Rexcel™) and powdered cellulose; calcium carbonate; glycine; bentonite; polyvinylpyrrolidone; and the like. Such diluents, if present, constitute in total about 5% to about 99%, preferably about 10% to about 85%, and more preferably about 20% to about 80%, of the total weight of the composition. The diluent or diluents selected preferably exhibit suitable flow properties and, where tablets are desired, compressibility. Lactose and microcrystalline cellulose, either individually or in combination, are preferred diluents. Both diluents are chemically compatible with AMG151. The use of extragranular microcrystalline cellulose (that is, microcrystalline cellulose added to a wet granulated composition after a drying step) can be used to improve hardness (for tablets) and/or disintegration time. Lactose, especially lactose monohydrate, is particularly preferred. Lactose typically provides compositions having suitable release rates of AMG151, stability, pre-compression flowability, and/or drying properties at a relatively low diluent cost. It provides a high density substrate that aids densification during granulation (where wet granulation is employed) and therefore improves blend flow properties.

Suspensions can be prepared similar to that described in Formulation technology: emulsions, suspensions, solid forms; Hans Mollet and Arnold Grubenmann; Pharmaceutical Emulsions and Suspensions; Fransiose Nielloud, CRC; 1st ed. (2000), or Pharmaceutical Dosage Forms: Disperse Systems, Vol 1 and Vol 2, Ed.: Herbert Lieberman, Martin Rieger, Gilbert Banker, Marcel Dekker, NY.

Compositions of the invention optionally comprise one or more pharmaceutically acceptable disintegrants as excipients, particularly for tablet formulations. Suitable disintegrants include, either individually or in combination, starches, including sodium starch glycolate (e.g., Explotab™ of PenWest) and pregelatinized corn starches (e.g., National™ 1551, National™ 1550, and Colorcon™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, locust bean, karaya, pectin and tragacanth gums. Disintegrants may be added at any suitable step during the preparation of the composition, particularly prior to granulation or during a lubrication step prior to compression. Such disintegrants, if present, constitute in total about 0.2% to about 30%, preferably about 0.2% to about 10%, and more preferably about 0.2% to about 5%, of the total weight of the composition. Croscarmellose sodium is a preferred disintegrant for tablet or capsule disintegration, and, if present, preferably constitutes about 0.2% to about 10%, more preferably about 0.2% to about 7%, and still more preferably about 0.2% to about 5%, of the total weight of the composition. Croscarmellose sodium confers superior intragranular disintegration capabilities to granulated compositions of the present invention.

The choice of formulation depends on various factors, such as the mode of drug administration (e.g., for oral administration, formulations in the form of tablets, pills or capsules are preferred) and the bioavailability of the drug substance. Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Other suitable pharmaceutical excipients and their formulations are described in Remington's Pharmaceutical Sciences, Gennaro, A. R. (Mack Publishing Company, 18th ed., 1995).

The level of the compound in a formulation can vary within the full range employed by those skilled in the art. Typically, the formulation contains, on a weight percent (wt %) basis, from about 0.01-99.99 wt % of a compound of the present invention based on the total formulation, with the balance being one or more suitable pharmaceutical excipients. Preferably, the compound is present at a level of about 1-80 wt %.

Compositions of the invention optionally comprise one or more pharmaceutically acceptable binding agents or adhesives as excipients, particularly for tablet formulations. Such binding agents and adhesives preferably impart sufficient cohesion to the powder being tableted to allow for normal processing operations such as sizing, lubrication, compression and packaging, but still allow the tablet to disintegrate and the composition to be absorbed upon ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™). Such binding agents and/or adhesives, if present, constitute in total about 0.5% to about 25%, preferably about 0.75% to about 15%, and more preferably about 1% to about 10%, of the total weight of the composition.

Compositions of the invention optionally comprise one or more pharmaceutically acceptable wetting agents as excipients. Such wetting agents are preferably selected to maintain the AMG151 crystalline forms in close association with water, a condition that is believed to improve bioavailability of the composition. Non-limiting examples of surfactants that can be used as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefosse), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% to about 15%, preferably about 0.4% to about 10%, and more preferably about 0.5% to about 5%, of the total weight of the composition. Wetting agents that are anionic surfactants are preferred. Sodium lauryl sulfate is a particularly preferred wetting agent. Sodium lauryl sulfate, if present, constitutes about 0.25% to about 7%, more preferably about 0.4% to about 4%, and still more preferably about 0.5% to about 2%, of the total weight of the composition.

Compositions of the invention optionally comprise one or more pharmaceutically acceptable lubricants (including anti-adherents and/or glidants) as excipients. Suitable lubricants include, either individually or in combination, glyceryl behapate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; sodium chloride; DL-leucine; PEG (e.g., Carbowax™ 4000 and Carbowax™ 6000); sodium oleate; sodium lauryl sulfate; and magnesium lauryl sulfate. Such lubricants, if present, constitute in total about 0.1% to about 10%, preferably about 0.2% to about 8%, and more preferably about 0.25% to about 5%, of the total weight of the composition. Magnesium stearate is a preferred lubricant used, for example, to reduce friction between the equipment and granulated mixture during compression of tablet formulations.

Suitable anti-adherents include talc, cornstarch, DL-leucine, sodium lauryl sulfate and metallic stearates. Talc is a preferred anti-adherent or glidant used, for example, to reduce formulation sticking to equipment surfaces and also to reduce static in the blend. Talc, if present, constitutes about 0.1% to about 10%, more preferably about 0.25% to about 5%, and still more preferably about 0.5% to about 2%, of the total weight of the composition.

Glidants can be used to promote powder flow of a solid formulation. Suitable glidants include colloidal silicon dioxide, starch, talc, tribasic calcium phosphate, powdered cellulose and magnesium trisilicate. Colloidal silicon dioxide is particularly preferred. Other excipients such as colorants, flavors and sweeteners are known in the pharmaceutical art and can be used in compositions of the present invention. Tablets can be coated, for example with an enteric coating, or uncoated. Compositions of the invention can further comprise, for example, buffering agents. Optionally, one or more effervescent agents can be used as disintegrants and/or to enhance organoleptic properties of compositions of the invention. When present in compositions of the invention to promote dosage form disintegration, one or more effervescent agents are preferably present in a total amount of about 30% to about 75%, and preferably about 45% to about 70%, for example about 60%, by weight of the composition.

An effervescent agent, present in a solid dosage form in an amount less than that effective to promote disintegration of the dosage form, provides improved dispersion of the AMG151 in an aqueous medium. When present in a pharmaceutical composition of the invention to promote intragastrointestinal dispersion but not to enhance disintegration, an effervescent agent is preferably present in an amount of about 1% to about 20%, more preferably about 2.5% to about 15%, and still more preferably about 5% to about 10%, by weight of the composition. An “effervescent agent” herein is an agent comprising one or more compounds which, acting together or individually, evolve a gas on contact with water. The gas evolved is generally oxygen or, most commonly, carbon dioxide. Preferred effervescent agents comprise an acid and a base that react in the presence of water to generate carbon dioxide gas. Preferably, the base comprises an alkali metal or alkaline earth metal carbonate or bicarbonate and the acid comprises an aliphatic carboxylic acid. Non-limiting examples of suitable bases as components of effervescent agents useful in the invention include carbonate salts (e.g., calcium carbonate), bicarbonate salts (e.g., sodium bicarbonate), sesquicarbonate salts, and mixtures thereof. Calcium carbonate is a preferred base. Non-limiting examples of suitable acids as components of effervescent agents useful in the invention include citric acid, tartaric acid, malic acid, adipic acid, succinic acid, acid anhydrides of such acids, acid salts of such acids, and mixtures thereof. Citric acid is a preferred acid. In a preferred embodiment of the invention, where the effervescent agent comprises an acid and a base, the weight ratio of the acid to the base is about 1:100 to about 100:1, more preferably about 1:50 to about 50:1, and still more preferably about 1:10 to about 10:1. In a further preferred embodiment of the invention, where the effervescent agent comprises an acid and a base, the ratio of the acid to the base is approximately stoichiometric.

For administration, the compounds of this invention are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulfuric acids, acacia, gelatin, sodium alginate, polyvinyl-pyrrolidine, and/or polyvinyl alcohol, and tableted or encapsulated for conventional administration. Alternatively, the compounds of this invention may be dissolved in saline, water, polyethylene glycol, propylene glycol, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The pharmaceutical compositions may be made up in a solid form (including granules, powders or suppositories) or in a liquid form (e.g., solutions, suspensions, or emulsions). The pharmaceutical compositions may be subjected to conventional pharmaceutical operations such as sterilization and/or may contain conventional adjuvants, such as preservatives, stabilizers, wetting agents, emulsifiers, buffers etc.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise, as in normal practice, additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting, sweetening, flavoring, and perfuming agents.

Solid dosage forms of the invention can be prepared by any suitable process, not limited to processes described herein. An illustrative process comprises (a) a step of blending crystalline forms of AMG151, or AMG151 drug substance, of the invention with one or more excipients to form a blend, and (b) a step of tableting or encapsulating the blend to form tablets or capsules respectively. In a preferred process, solid dosage forms are prepared by a process comprising (a) a step of blending a form of AMG151, or AMG151 drug substance, of the invention with one or more excipients to form a blend, (b) a step of granulating the blend to form a granulate, and (c) a step of tableting or encapsulating the blend to form tablets or capsules respectively. Step (b) can be accomplished by any dry or wet granulation technique known in the art, but is preferably a wet granulation step followed by a step of drying the resulting granulate prior to tableting or encapsulating. One or more diluents, one or more disintegrants and one or more binding agents are preferably added, for example in the blending step, a wetting agent can optionally be added, for example in the granulating step, and one or more disintegrants are preferably added after granulating but before tableting or encapsulating. A lubricant is preferably added before tableting. Blending and granulating can be performed independently under low or high shear. A process is preferably selected that forms a granulate that is uniform in drug content, that readily disintegrates, that flows with sufficient ease so that weight variation can be reliably controlled during capsule filling or tableting, and that is dense enough in bulk so that a batch can be processed in the selected equipment and individual doses fit into the specified capsules or tablet dies.

Conventional tableting and encapsulation techniques known in the art can be employed. Where coated tablets are desired, conventional coating techniques are suitable. Any tablet hardness convenient with respect to handling, manufacture, storage and ingestion can be employed. The material to be tableted, however, should not be compressed to such a degree that there is subsequent difficulty in achieving hydration when exposed to gastric fluid.

AMG151 dosage forms of the invention preferably comprise AMG151 in a daily dosage amount of about 0.1 mg to about 500 mg, more preferably about 1 mg to about 250 mg, and most preferably about 10 mg to about 175 mg. Compositions of the invention comprise one or more orally deliverable dose units. Each dose unit comprises AMG151 in a therapeutically effective amount that is preferably about 10 mg to about 500 mg. The term “dose unit” herein means a portion of a pharmaceutical composition that contains an amount of a therapeutic or prophylactic agent, in the present case AMG151, suitable for a single oral administration to provide a therapeutic effect. Typically one dose unit, or a small plurality (up to about 4) of dose units, in a single administration provides a dose comprising a sufficient amount of the agent to result in the desired effect. Administration of such doses can be repeated as required, typically at a dosage frequency of 1 to about 4 times per day. It will be understood that a therapeutically effective amount of AMG151 for a subject is dependent inter alia on the body weight of the subject. A “subject” herein to which a therapeutic agent or composition thereof can be administered includes a human patient of either sex and of any age, and also includes any nonhuman animal, particularly a warm-blooded animal, more particularly a domestic or companion animal, illustratively a cat, dog or horse. When the subject is a child or a small animal (e.g., a dog), for example, an amount of AMG151 relatively low in the preferred range of about 10 mg to about 500 mg is likely to provide blood serum concentrations consistent with therapeutic effectiveness. Where the subject is an adult human or a large animal (e.g., a horse), achievement of such blood serum concentrations of AMG151 are likely to require dose units containing a relatively greater amount of AMG151. Typical dose units in a composition of the invention contain about 10, 20, 25, 37.5, 50, 75, 100, 125, 150, 175, 200, 250, 300, 350, 400, 450 or 500 mg of AMG151. For an adult human, a therapeutically effective amount of AMG151 per dose unit in a composition of the present invention is typically about 50 mg to about 400 mg. Especially preferred amounts of AMG151 per dose unit are about 200 mg to about 500 mg, for example about 350, about 400 or about 450 mg. A dose unit containing a particular amount of AMG151 can be selected to accommodate any desired frequency of administration used to achieve a desired daily dosage. The daily dosage and frequency of administration, and therefore the selection of appropriate dose unit, depends on a variety of factors, including the age, weight, sex and medical condition of the subject, and the nature and severity of the condition or disorder, and thus may vary widely. The term “oral administration” herein includes any form of delivery of a therapeutic agent or a composition thereof to a subject wherein the agent or composition is placed in the mouth of the subject, whether or not the agent or composition is immediately swallowed. Thus “oral administration” includes buccal and sublingual as well as esophageal administration. Absorption of the agent can occur in any part or parts of the gastrointestinal tract including the mouth, esophagus, stomach, duodenum, ileum and colon. The term “orally deliverable” herein means suitable for oral administration.

Such compositions are useful in treatment of diabetes in a subject.

The present invention is further directed to a therapeutic method of treating a condition or disorder where treatment with an anti-diabetes drug is indicated, the method comprising oral administration of a composition of the invention to a subject in need thereof. The dosage regimen to prevent, give relief from, or ameliorate the condition or disorder preferably corresponds to once-a-day or twice-a-day treatment, but can be modified in accordance with a variety of factors. These include the type, age, weight, sex, diet and medical condition of the subject and the nature and severity of the disorder. Thus, the dosage regimen actually employed can vary widely and can therefore deviate from the preferred dosage regimens set forth above.

Initial treatment can begin with a dose regimen as indicated above. Treatment is generally continued as necessary over a period of several weeks to several months or years until the condition or disorder has been controlled or eliminated. Subjects undergoing treatment with a composition of the invention can be routinely monitored by any of the methods well known in the art to determine effectiveness of therapy. Continuous analysis of data from such monitoring permits modification of the treatment regimen during therapy so that optimally effective doses are administered at any point in time, and so that the duration of treatment can be determined. In this way, the treatment regimen and dosing schedule can be rationally modified over the course of therapy so that the lowest amount of the composition exhibiting satisfactory effectiveness is administered, and so that administration is continued only for so long as is necessary to successfully treat the condition or disorder.

The forms of the present invention can be used as prophylactics or therapeutic agents for treating diseases or disorders mediated by deficient levels of glucokinase activity or which can be treated by activating glucokinase including, but not limited to, diabetes mellitus, impaired glucose tolerance, IFG (impaired fasting glucose) and IFG (impaired fasting glycemia), as well as other diseases and disorders such as those discussed below. Furthermore, the compounds of the present invention can be also used to prevent the progression of the borderline type, impaired glucose tolerance, IFG (impaired fasting glucose) or IFG (impaired fasting glycemia) to diabetes mellitus.

In one embodiment, the forms of the present invention are useful for the therapeutic treatment of diseases or disorders mediated by deficient levels of glucokinase activity or which can be treated by activating glucokinase including, but not limited to, diabetes mellitus, impaired glucose tolerance, IFG (impaired fasting glucose) and IFG (impaired fasting glycemia), as well as other diseases and disorders such as those discussed below.

Accordingly, another aspect of the invention provides methods of treating or preventing diseases or conditions described herein by administering to a mammal, such as a human, a therapeutically effective amount of a crystalline form of AMG151.

The phrase “therapeutically effective amount” means an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The amount of AMG151 that will correspond to such an amount will vary depending upon factors such as the particular compound, disease condition and its severity, the identity (e.g., weight) of the mammal in need of treatment, but can nevertheless be routinely determined by one skilled in the art.

The terms “treat” and “treatment” refer to both therapeutic treatment and prophylactic measures, wherein the object is to slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be slowed down or alleviated.

In one embodiment, the terms “treat” and “treatment” refer to therapeutic treatment, wherein the object is to slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder or those in which the condition or disorder is to be slowed down or alleviated.

The terms prevent” or “preventing” refer to prophylactic treatment. As used herein the terms “prevent” or “preventing” means the prevention of the onset, recurrence or spread, in whole or in part, of the disease or condition as described herein, or a symptom thereof.

As used herein, the term “mammal” refers to a warm-blooded animal that has or is at risk of developing a disease described herein and includes, but is not limited to, guinea pigs, dogs, cats, rats, mice, hamsters, and primates, including humans.

In certain embodiments, the methods of this invention are useful for treating diabetes mellitus. Diabetes mellitus is a condition where the fasting plasma glucose level (glucose concentration in venous plasma) is greater than or equal to 126 mg/dL (tested on two occasions) and the 2-hour plasma glucose level of a 75 g oral glucose tolerance test (OGTT) is greater than or equal to 200 mg/dL. Additional classic symptoms include polydipsia, polyphagia and polyuria. In one embodiment, the treatment is therapeutic treatment.

In certain embodiments, the methods of this invention are useful for treating the syndrome of impaired glucose tolerance (IGT). IGT is diagnosed by the presentation of a fasting plasma glucose level of less than 126 mg/dL and a 2-hour post-oral glucose challenge level greater than 140 mg/dL. In one embodiment, the treatment is therapeutic treatment.

The forms of the present invention can be also used as prophylactics or therapeutic agents of diabetic complications such as, but not limited to, neuropathy, nephropathy, retinopathy, cataract, macroangiopathy, osteopenia, diabetic hyperosmolar coma, infectious diseases (e.g., respiratory infection, urinary tract infection, gastrointestinal tract infection, dermal soft tissue infection, lower limb infection etc.), diabetic gangrene, xerostomia, decreased sense of hearing, cerebrovascular disease, peripheral circulatory disturbance, etc.

The forms of the present invention can be also used as prophylactics or therapeutic agents in the treatment of diseases and disorders such as, but not limited to, obesity, metabolic syndrome (syndrome X), hyperinsulinemia, hyperinsulinemia-induced sensory disorder, dyslipoproteinemia (abnormal lipoproteins in the blood) including diabetic dyslipidemia, hyperlipidemia, hyperlipoproteinemia (excess of lipoproteins in the blood) including type I, II-a (hypercholesterolemia), II-b, III, IV (hypertriglyceridemia) and V (hypertriglyceridemia), low HDL levels, high LDL levels, atherosclerosis and its sequelae, vascular restenosis, neurodegenerative disease, depression, CNS disorders, liver steatosis, osteoporosis, hypertension, renal diseases (e.g., diabetic nephropathy, glomerular nephritis, glomerulosclerosis, nephrotic syndrome, hypertensive nephrosclerosis, terminal renal disorder etc.), myocardiac infarction, angina pectoris, and cerebrovascular disease (e.g., cerebral infarction, cerebral apoplexy).

Treatment of diseases and disorders herein is intended to also include the prophylactic administration of a form of the invention, a pharmaceutical salt thereof, or a pharmaceutical composition of either to a subject (i.e., an animal, preferably a mammal, most preferably a human) believed to be in need of preventative treatment.

The dosage regimen for treating diabetes, and/or hyperglycemia with the forms of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods. Dosage levels of the order from about 0.01 mg to 30 mg per kilogram of body weight per day, preferably from about 0.1 mg to 10 mg/kg, more preferably from about 0.25 mg to 1 mg/kg are useful for all methods of use disclosed herein.

The pharmaceutically active forms of this invention can be processed in accordance with conventional methods of pharmacy to produce medicinal agents for administration to patients, including humans and other mammals.

For oral administration, the pharmaceutical composition may be in the form of, for example, a capsule, a tablet, a suspension, or liquid. The pharmaceutical composition is preferably made in the form of a dosage unit containing a given amount of the active ingredient. For example, these may contain an amount of active ingredient from about 1 to 2000 mg, preferably from about 10 to 1000 mg, more preferably from about 50 to 500 mg. A suitable daily dose for a human or other mammal may vary widely depending on the condition of the patient and other factors, but, once again, can be determined using routine methods.

Combinations

While the forms of the invention can be administered as the sole active pharmaceutical agent, they can also be used in combination with one or more forms of the invention or other agents. When administered as a combination, the therapeutic agents can be formulated as separate compositions that are administered at the same time or sequentially at different times, or the therapeutic agents can be given as a single composition.

The phrase “co-therapy” (or “combination-therapy”), in defining use of a form of the present invention and another pharmaceutical agent, is intended to embrace administration of each agent in a sequential manner in a regimen that will provide beneficial effects of the drug combination, and is intended as well to embrace co-administration of these agents in a substantially simultaneous manner, such as in a single capsule having a fixed ratio of these active agents or in multiple, separate capsules for each agent.

Specifically, the administration of forms of the present invention may be in conjunction with additional therapies known to those skilled in the art in the treatment of diabetes. The forms of the present invention can be used in combination with one or more additional drugs, for example a compound that works by the same or a different mechanism of action, such as insulin preparations, agents for improving insulin resistance, alpha-glucosidase inhibitors, biguanides, insulin secretagogues, dipeptidylpeptidase IV (DPP IV) inhibitors, beta-3 agonists, amylin agonists, phosphotyrosine phosphatase inhibitors, gluconeogenesis inhibitors, sodium-glucose co-transporter inhibitors, known therapeutic agents for diabetic complications, antihyperlipidemic agents, hypotensive agents, and antiobesity agents. An example of an agent for improving insulin resistance is an agonist for peroxisome proliferator-activated receptor-gamma (PPAR gamma).

If formulated as a fixed dose, such combination products employ the forms of this invention within the accepted dosage ranges. The forms of the invention may also be administered sequentially with known anti-diabetes agents when a combination formulation is inappropriate. The invention is not limited in the sequence of administration; forms of the invention may be administered either prior to, simultaneous with or subsequent to administration of the known anti-diabetes agent.

If the patient is to receive or is receiving multiple pharmaceutically active compounds, the compounds can be administered simultaneously, or sequentially. For example, in the case of tablets, the active compounds may be found in one tablet or in separate tablets, which can be administered at once or sequentially in any order. In addition, it should be recognized that the compositions may be different forms. For example, one or more compounds may be delivered via a tablet, while another is administered via injection or orally as a syrup. All combinations, delivery methods and administration sequences are contemplated. The forms of the present invention may be used in the manufacture of a medicament for the treatment of a disease and/or condition mediated by GKA, such as type 2 diabetes.

The forms of the present invention may be used in combination with other pharmaceutically active compounds. It is noted that the term “pharmaceutically active compounds” can include biologics, such as proteins, antibodies and peptibodies. Examples of other pharmaceutically active compounds include, but are not limited to: (a) dipeptidyl peptidase IV (DPP-IV) inhibitors such as Vildagliptin (Novartis), Sitagliptin (Merck&Co.), Saxagliptin (BMS) Allogliptin (Takeda); (b) insulin sensitizers including (i) PPARγ agonists such as the glitazones (e.g., troglitazone, pioglitazone, edaglitazone, rosiglitazone, and the like) and other PPAR ligands, including PPARα/γ dual agonists such as muraglitazar (BMS) and tesaglitazar (AstraZeneca), and PPARα agonists such as fenofibric acid derivatives (gemfibrozil, clofibrate, fenofibrate and bezafibrate), (ii) biguanides such as metformin and phenformin, and (iii) protein tyrosine phosphatase-1B (PTP-1B) inhibitors; (c) insulin or insulin mimetics; (d) incretin and incretin mimetics such as (i) Exenatide available from Amylin Pharmaceuticals, (i) amylin and amylin mimetics such as pramlintide acetate, available as Symlin®, (iii) GLP-1, GLP-1 mimetics, and GLP-1 receptor agonists, (iv) GIP, GIP mimetics and GIP receptor agonists; (e) sulfonylureas and other insulin secretagogues, such as tolbutamide, glyburide, gliclazide, glipizide, glimepiride, meglitinides, and repaglinide; (f) α-glucosidase inhibitors (such as acarbose and miglitol); (g) glucagon receptor antagonists; (h) PACAP, PACAP mimetics, and PACAP receptor agonists; (i) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) bile acid sequestrants such as cholestyramine, colestipol and dialkylaminoalkyl derivatives of a cross-linked dextran, (iii) nicotinyl alcohol, nicotinic acid or a salt thereof, (iv) PPARα agonists such as fenofibric acid derivatives (gemfibrozil, clofibrate, fenofibrate and bezafibrate), (v) PPARα/γ dual agonists such as muraglitazar (BMS) and tesaglitazar (AstraZeneca), (vi) inhibitors of cholesterol absorption, such as beta-sitosterol and ezetimibe, (vii) acyl CoA: cholesterol acyltransferase inhibitors such as avasimibe, and (viii) anti-oxidants such as probucol; (j) PPARγ agonists such as GW-501516 from GSK; (k) anti-obesity compounds such as fenfluramine, dexfenfluramine, phentemine, sibutramine, orlistat, neuropeptide Y1 or Y5 antagonists, MTP inhibitors, squalene synthase inhibitor, lipoxygenase inhibitor, ACAT inhibitor, Neuropeptide Cannabinoid CB-1 receptor antagonists, CB-1 receptor inverse agonists and antagonists, fatty acid oxidation inhibitors, appetite suppressants (l) adrenergic receptor agonists, melanocortin receptor agonists, in particular—melanocortin-4 receptor agonists, ghrelin antagonists, and melanin-concentrating hormone (MCH) receptor antagonists; (m) ileal bile acid transporter inhibitors; (n) agents intended for use in inflammatory conditions such as aspirin, non-steroidal anti-inflammatory drugs, glucocorticoids, azalfidine, and selective cyclooxygenase-2 inhibitors; (o) antihypertensive agents such as ACE inhibitors (enalapril, lisinopril, captopril, quinapril, fosinoprol, ramipril, spirapril, tandolapril), angiotensin-II (AT-1) receptor blockers (losartan, candesartan, irbesartan, valsartan, telmisartan, eprosartan), beta blockers and calcium channel blockers; and (p) glucokinase activators (GKAs); (q) agents which can be used for the prevention, delay of progression or treatment of neurodegenerative disorders, cognitive disorders or a drug for improving memory such as anti-inflammatory drugs, antioxidants, neuroprotective agents, glutamate receptor antagonists, acetylcholine esterase inhibitors, butyrylcholinesterase inhibitors, MAO inhibitors, dopamine agonists or antagonists, inhibitors of gamma and beta secretases, inhibitors of amyloid aggregation, amyloid beta peptide, antibodies to amyloid beta peptide, inhibitors of acetylcholinesterase, glucokinase activators, agents directed at modulating GABA, NMDA, cannabinoid, AMPA, kainate, phosphodiesterase (PDE), PKA, PKC, CREB or nootropic systems; (r) leukocyte growth promotors intended for the treatment and prevention of reduced bone marrow production, infectious diseases, hormone dependent disorders, inflammatory diseases, HIV, allergies, leukocytopenia, and rheumatism; (s) SGLT2 inhibitor; (t) glycogen phosphorylase inhibitor; (u) aP2 inhibitors; (v) aminopeptidase N inhibitor (w) vasopeptidase inhibitors like neprilysin inhibitors and/or ACE inhibitors or dual NEP/ACE inhibitor; (x) growth hormone secretagogue for enhancing growth hormone levels and for treating growth retardation/dwarfism or metabolic disorders or where the disorder is an injury, or a wound in need of healing, or a mammalian patient recovering from surgery; (y) 5-HT 3 or 5-HT 4 receptor modulators (tegaserod, cisapride, nor-cisapride, renzapride, zacopride, mosapride, prucalopride, buspirone, norcisapride, cilansetron, ramosetron, azasetron, ondansetron, etc.); (Za) aldose reductase inhibitors; (Zb) sorbitol dehydrogenase inhibitors; (Zc) AGE inhibitors; (Zd) erythropoietin agonist such as EPO, EPO mimetics, and EPO receptor agonists. The forms of the present invention may also be used in combination with GPR40 agonists.

The compound to be administered in combination with AMG151 can be formulated separately from the AMG151 or co-formulated with the AMG151 in a composition of the invention. Where AMG151 is co-formulated with a second drug, the second drug can be formulated in immediate-release, rapid-onset, sustained-release or dual-release form.

Processes for preparing AMG151 are set forth herein as well as in U.S. Pat. Nos. 8,022,223 and 8,212,045, incorporated herein by reference.

X-ray Powder Diffraction

X-ray powder diffraction data was obtained using the Phillips x-ray automated powder diffractometer (X'Pert) equipped with a fixed slit. The radiation was CuKa (1.541837A) and the voltage and current were 45 kV and 40 mA, respectively. Data was collected at room temperature from 3.000 to 40.000 degree 2-theta; step size was 0.008 degrees; counting time was 15.240 seconds. Samples ranging from 5-40 mg were prepared on the sample holder and the stage was rotated at a revolution time of 2.000 seconds. An average error/standard deviation is 0.2-0.5 2-theta.

Thermal Analysis

The thermal properties of AMG151 samples were characterized using a DSC Q1000 or DSC Q 100, TA Instruments, differential scanning calorimetry, and a TGA Q 500, TA Instruments, thermogravimetric analyzer. Data analysis was performed utilizing Universal Analysis 2000, TA Instruments. Heating rates of 10° C./min were used over a variety of temperature ranges for differential scanning calorimetry and thermogravimetric analysis. Samples ranging from <1-5 mg were prepared in crimped or open aluminum pans for DSC analysis.

Moisture Balance

Moisture balance data was collected using a VTI SGA 100 or SGA CX symmetrical vapor sorption analyzer. Relative humidity was varied in increments or 5%, starting at 5% relative humidity thereby increasing to 95% relative humidity, and then undergoing a drying cycle back to 5% relative humidity. Equilibrium criteria was set at 0.01% weight change in 1 minute with a max equilibrium time of 180 minutes. Approximately 1-15 mg of sample was used.

The following examples illustrate aspects of the present invention but are not to be construed as limitations.

### Example 1

### Preparation of (S)-1-(5-((3 ((2-methylpyridin-3-yl)oxy)-5-(pyridin-2-ylthio)pyridin-2-yl)amino)-1,2,4-thiadiazol-3-yl)ethane-1,2-diol

### Reactants:

| | Amount | Vol | | Moles | Molarity |
|---|---|---|---|---|---|
| Name | (mg) | (mL) | Eq | (mmol) | (molar) |
| 3-((2-methylpyridin-3-yl)oxy)-5- | 106 | | 1.000 | 0.340 | |
| (pyridin-2-ylthio)pyridine 1-oxide | | | | | |
| DIPEA | 88 | 0.119 | 2 | 0.680 | |
| Tosyl-Cl | 78 | | 1.2 | 0.408 | |
| hydrochloric acid | 1200 | 1 mL | 5.89 | 2.000 | 2 |
| (S)-tert-butyl (3-(1,4- | 116 | | 1.000 | 0.340 | |
| dioxaspiro[4.5]decan-2-yl)-1,2,4- | | | | | |
| thiadiazol-5-yl)carbamate | | | | | |

| | | |
|---|---|---|
| | mL/g | Solvent Volume |
| Solvent Name | (mL/g) | (mL) |

| DCM | 10 | | 1.160 | | | |
|---|---|---|---|---|---|---|
| | Theo | Actual | | | Actual | |
| | Mass | Mass | Yield | | Mol | |
| Name | (mg) | (mg) | (%) | MW | (mmol) | Eq. |
| (S)-1-(5-((3-((2-methylpyridin-3- | 154 | 67 | 43.4 | 454.525 | 0.147 | 1.000 |
| yl)oxy)-5-(pridin-2-ylthio)pyridin- | | | | | | |
| 2-yl)amino)-1,2,4-thiadiazol-3- | | | | | | |
| yl)ethane-1,2-diol | | | | | | |

3-((2-methylpyridin-3-yl)oxy)-5-(pyridin-2-ylthio)pyridine 1-oxide and (S)-tert-butyl (3-(1,4-dioxaspiro[4.5]decan-2-yl)-1,2,4-thiadiazol-5-yl)carbamate in 1 mL of DCM. DIPEA was added then cooled in an ice-bath to 0° C. Added tosyl chloride as a solid in one portion. Removed reaction from ice-bath. Evaporated off DCM using a stream of N₂ then added 2N HCl (10 Vol, 1 mL) and heated to 60° C. Added 2 mL of 1N KOAc. White amorphous suspension formed. Added 1 mL n-propanol which dissolved the solid. Heated to 60° C. to dissolve the solid then seeded with (AMG151 Form F). A white slurry forms. Adjusted pH to 5.0 by adding 0.1 mL of 10 N NaOH. Filtered and washed with 1 ml of water. Dried solid on frit using vacuum. Obtained 67 mg of the product.

### Example 2

### Synthesis of AMG151 Free Base

| Material | Supplier | Equiv./Volume | | Moles | Theoretical | |
|---|---|---|---|---|---|---|
| 3-((2-methylpyridin-3-yl)oxy)-5- | | 1.0 | | 29.0 | 9.0 | kg |
| (pyridin-2-ylthio)pyridin-2-amine | | | | | | |
| THF (anhydrous) | Sigma Aldrich | 8.0 | V | — | 72.0 | L |
| potassium tert-butoxide | BASF | 2.30 | | 66.7 | 7.48 | kg |
| THF (anhydrous) | Sigma Aldrich | 1.5 | V | — | 13.5 | L |
| 3-[(4S)-2,2-dimethyl-1,3- | | 1.20 | | 34.8 | 11.8 | kg |
| dioxolan-4-yl]-5-[(4- | | | | | | |
| methylphenyl)sulfonyl]- | | | | | | |
| 1,2,4-thiadiazole | | | | | | |
| THF (anhydrous) | Sigma Aldrich | 0.5 | V | — | 4.5 | L |
| Water | | 15 | V | — | 135 | L |
| Ammonium chloride | JT Baker | 32 | | — | 928 | kg |
| Water | | 1.0 | V | — | 9.0 | L |
| Toluene | Sigma Aldrich | 15.5 | V | — | 139.5 | L |
| (S)-3-(2,2-dimethyl-1,3-dioxolan- | | 0.001 | | 0.029 | 14 | g |
| 4-yl)-N-(3-((2-methylpyridin-3- | | | | | | |
| yl)oxy)-5-(pyridin-2- | | | | | | |
| ylthio)pyridin-2-yl)-1,2,4- | | | | | | |
| thiadiazol-5-amine (seed) | | | | | | |
| Heptane | Sigma Aldrich | 5.0 | | — | 45.0 | L |
| 1:1 Toluene:Heptane (rinse) | — | 5.0 | V | — | 45.0 | L |

Set jacket of 400 L jacketed reactor to 25±5° C.

Charge 3-((2-methylpyridin-3-yl)oxy)-5-(pyridin-2-ylthio)pyridin-2-amine to 400 L jacketed reactor as a solid.

Inert the contents of 400 L jacketed reactor by vacuum-nitrogen refill (3 cycles).

Initiate slow agitation in the 400 L jacketed reactor.

Charge 8.0 vol of anhydrous THF to 400 L jacketed reactor while maintaining batch temperature less than 35° C.

Charge potassium tert-butoxide to 400 L jacketed reactor as a solid maintaining a batch temperature of less than 30° C. Dissolution is mildly exothermic.

Agitate at 25±5° C. for at least 15 min. Ensure homogenous mixture formed.

Adjust the contents of 400 L jacketed reactor to 0±5° C.

Charge 3-[(4S)-2,2-dimethyl-1,3-dioxolan-4-yl]-5-[(4-methylphenyl)sulfonyl]-1,2,4-thiadiazole to 250 L jacketed reactor.

Charge 1.5 vol of anhydrous THF to 250 L jacketed reactor.

Initiate agitation in 250 L jacketed reactor: Dissolution is fast and endothermic.

Inert the contents of 250 L jacketed reactor by vacuum-nitrogen refill (3 cycles).

Transfer the sulfone solution from 250 L jacketed reactor to 400 L jacketed reactor maintaining internal temperature of 400 L jacketed reactor at 0±5° C. Addition is exothermic.

Charge 0.5 vol of anhydrous THF to 250 L jacketed reactor vessel.

Stir the rinse in 250 L jacketed reactor at least 1 minutes at 25±5° C.

Transfer contents of 250 L jacketed reactor to 400 L jacketed reactor maintaining internal temperature of 400 L jacketed reactor at 0±5° C.

Charge 5.0 vol of saturated aqueous ammonium chloride to 400 L jacketed reactor.

Charge 1.0 vol of DI water to 400 L jacketed reactor.

Adjust contents of 400 L jacketed reactor to 25±5° C.

Agitate at 25±5° C. for about 5 min. Allow the contents of 400 L jacketed reactor to settle.

Drain the lower (aqueous) layer from 400 L jacketed reactor.

Charge 5.0 vol of saturated aqueous ammonium chloride to 400 L jacketed reactor at 25±5° C.

Agitate at 25±5° C. for approximately 5 min.

Allow the contents of 400 L jacketed reactor to settle.

Drain the lower (aqueous) layer from 400 L jacketed reactor.

Charge 5.0 vol of saturated aqueous ammonium chloride to 400 L jacketed reactor at 25±5° C.

Agitate at 25±5° C. for about 5 min. Allow the contents of 400 L jacketed reactor to settle.

Drain the lower (aqueous) layer from 400 L jacketed reactor.

Charge 5.0 vol of toluene to 400 L jacketed reactor.

Agitate at 25±5° C. for about 5 min. Allow the contents of 400 L jacketed reactor to settle.

Drain and discard the lower (aqueous) layer from 400 L jacketed reactor.

Set jacket in 400 L jacketed reactor to 50-80° C.

Distill contents of 400 L jacketed reactor under reduced pressure (≦100 Torr). Target: 5-6 vol remaining in 400 L jacketed reactor. Add BHT to distillate.

Charge 5 vol toluene to 400 L jacketed reactor.

Distill contents of 400 L jacketed reactor under reduced pressure (≦100 Torr). Target: 5-6 vol remaining in 400 L jacketed reactor. Add BHT to distillate.

Charge 5 vol toluene to 400 L jacketed reactor.

Adjust contents of 400 L jacketed reactor to 50±5° C.

Set jacket of 250 L jacketed reactor to 50±5° C.

Transfer contents of 400 L jacketed reactor through an inline 5 μM filter to clean, dry 250 L jacketed reactor.

Charge toluene rinse to 400 L jacketed reactor.

Stir rinse in 400 L jacketed reactor until 50±5° C.

Transfer rinse in 400 L jacketed reactor through inline 5 μM filter to 250 L jacketed reactor.

Distill contents of 250 L jacketed reactor under reduced pressure (≦100 Torr). Target: 5-6 vol remaining in 250 L jacketed reactor.

Crystallization

Adjust contents of 250 L jacketed reactor to 50±3° C.

Dissolve (S)-3-(2,2-dimethyl-1,3-dioxolan-4-yl)-N-(3-((2-methylpyridin-3-yl)oxy)-5-(pyridin-2-ylthio)pyridin-2-yl)-1,2,4-thiadiazol-5-amine (seed slurry) in heptane.

Add the “seed slurry” to the 250 L jacketed reactor.

Adjust internal temperature of 250 L jacketed reactor from 50±3° C. to 20±3° C. over ≧5 hours.

Agitate contents of 250 L jacketed reactor at 20±3° C. for ≧1 hour.

Charge heptane to 250 L jacketed reactor at 20±3° C. over ≧3 hours until 50:50 v/v toluene:heptane is obtained. 50:50 v/v toluene:heptane is desired.

Age contents of 250 L jacketed reactor at 20±3° C. for ≧1 hour.

Filter contents of 250 L jacketed reactor on Aurora filter (10-15 μm PTFE cloth), collecting the filtrate in a suitable vessel.

Charge 2.5 vol toluene to a clean vessel.

Charge 2.5 vol heptane to the same vessel.

Charge the toluene:heptane 1:1 v/v rinse to 250 L jacketed reactor.

Stir contents of 250 L jacketed reactor at 20±5° C. for ≧5 min.

Transfer contents of 250 L jacketed reactor to filter cake.

Dry filter cake for at least 4 hours under N₂ and vacuum.

| Material | Equivalents/Volumes | | Moles | Mass | Volume |
|---|---|---|---|---|---|
| (S)-3-(2,2-dimethyl-1,3- | 1.0 | equiv | 21.67 | 10.72 kg | — |
| dioxolan-4-yl)-N-(3-((2-methyl- | | | | | |
| pyridin-3-yl)oxy)-5-(pyridin- | | | | | |
| 2-ylthio)pyridin-2-yl)-1,2,4- | | | | | |
| thiadiazol-5-amine | | | | | |
| DI Water | 3.0 | Vol | — | 32.3 kg | 32.2 L |
| 1.0N Hydrochloric acid | 3.0 | equiv | — | 66.1 kg | 65.0 L |
| 1.0N Potassium Acetate Solution | 1.5 | equiv | 32.5 | — | 32.5 L |
| AMG151•H₂O (seed) | 0.03 | equiv | — | 0.32 kg | — |
| DI water | 0.12 | Vol | — | 1.3 kg | 1.3 L |
| 1.0N Potassium Acetate Solution | 1.6 | equiv | 34.7 | — | 34.7 L |
| DI Water | 0.010 | Vol | — | 21.4 kg | 21.4 L |
| DI Water | 11.0 | Vol | — | 21.4 kg | 21.4 L |

Set the jacket temperature of 250 L jacketed reactor to 25° C.

Charge 1N HCl (66.1 kg) to the 250 L jacketed reactor.

Initiate agitation in the 250 L jacketed reactor.

Charge (S)-3-(2,2-dimethyl-1,3-dioxolan-4-yl)-N-(3-((2-methylpyridin-3-yl)oxy)-5-(pyridin-2-ylthio)pyridin-2-yl)-1,2,4-thiadiazol-5-amine (10.718 kg) to the 250 L jacketed reactor as a solid. Dissolution is mildly exothermic.

Initiate N₂ sweep in the 250 L jacketed reactor.

Agitate batch in 250 L jacketed reactor at 25±5° C. for ≧5 h to form (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol.

The (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol is used in the next step (Example 3) without further processing.

### Example 3

### Preparation of AMG151 Hydrate

Set the jacket temperature of the 400 L jacketed reactor to 25° C.

Transfer the solution of (15)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol from Example 2 through a Poly Ethylene, Poly Propylene, or PTFE inline 5 μM filter to the clean, dry 400 L jacketed reactor.

Charge water (32 L) to the 250 L jacketed reactor as a rinse.

Stir the rinse in the 250 L jacketed reactor for at least 1 minute.

Transfer contents of 250 L jacketed reactor through the inline 5 μM filter to the 400 L jacketed reactor.

Initiate N₂ sweep and agitation in the 400 L jacketed reactor.

Adjust batch temperature in 400 L jacketed reactor to 25±5° C.

Polish filter the 1.0N Aq KOAc (67 L) through a PE, PTFE, or Nylon line filter (5 μm).

Add 1.5±0.05 equivalent of the filtered 1.0N Aq KOAc solution (32.5 L) to the 400 L jacketed reactor over ≧40 minutes while maintaining a batch temperature of 25±5° C.

Charge AMG151 hydrate (0.32 kg) to an appropriate container labeled “Seed Slurry”.

Charge DI water (1 L) to “Seed Slurry”.

Charge “Seed Slurry” to the 400 L jacketed reactor as a slurry.

Agitate batch at 25±5° C. for ≧120 min.

Charge 1.6±0.1 equivalent of the filtered 1.0N Aq KOAc solution (35 L) to the 400 L jacketed reactor over ≧8 h while maintaining a batch temperature of 25±5° C. The pH is typically around 3.1.

Agitate batch at 25±5° C. for >2 hours.

Filter contents of the 400 L jacketed reactor on a 20″ Aurora filter fitted with a 10-15 μm PTFE cloth, collecting the filtrate in an appropriate vessel.

Charge DI Water (21 L) to the 400 L jacketed reactor.

Stir batch in the 400 L jacketed reactor at 20±5° C. for ≧5 min.

Transfer contents of 400 L jacketed reactor to filter cake, collecting the wash in a suitable vessel.

Charge DI Water (21 L) to the 400 L jacketed reactor.

Stir the batch in the 400 L jacketed reactor at 20±5° C. for ≧5 min.

Transfer contents of 400 L jacketed reactor to filter cake, collecting the wash in a suitable vessel.

Dewater the filter cake using N₂ and vacuum

Transfer the wetcake to the Double Cone Dryer.

Set the Double Cone Dryer k to 40° C., and dry the cake at reduced pressure.

### Example 4

### Preparation of 3-(2-methylpyridin-3-yloxy)-5-(pyridin-2-ylthio)pyridine 1-oxide

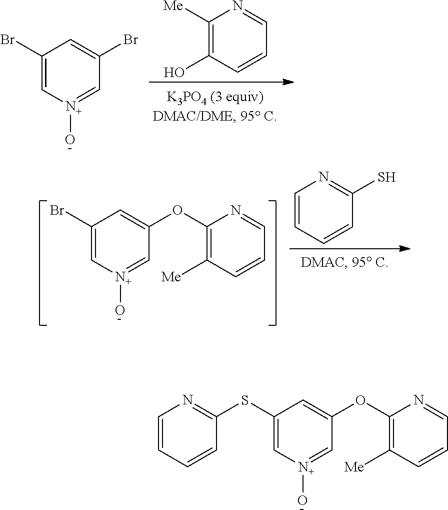

Reagents:

3,5-dibromopyridine (MW 252.89): 3.496 kg (13.82 mol, 1.0 equivalents), purchased from Astatech (CAS #625-92-3);

2-methylpyridin-3-ol (MW 109.13): 1.509 kg (13.83 mol, 1.0 equivalents), purchased from Irix (CAS #1121-25-1);

pyrine-2-thiol (MW 111.16): 1.690 kg (15.20 mol, 1.40 equivalents), purchased from Irix (CAS #2637-34-5);

DMAc: 4.929 kg (5.24 mL, 1.5 equivalents), purchased from Aldrich (CAS #127-19-5);

DME: 13.690 kg (15.74 mL, 4.50 equivalents), purchased from Aldrich (CAS #110-71-4; K₃PO₄ (MW 212.27): 3.521 kg (16.59 mol; 1.30 equivalents), purchased from Stern (CAS #7778-53-2);

K₃PO₄ (MW 212.27): 3.521 kg (16.59 mol; 1.40 equivalents), purchased from Stern (CAS #7778-53-2).

Process

Clean and Inspect: 60 L jacketed reactor. Dry under nitrogen.

Cool the condenser to 5° C.

Charge 3493 g (13.82 mol, 1.0 equivalent) of 3,5-dibromopyridine N-oxide to the 60 L reactor.

Charge 1505 g (13.82 mol, 1.0 equivalent) of 2-methylpyridin-3-ol to the 60 L reactor.

Charge 13,630 g (4.5V) of DME to the 60 L reactor.

Initiate agitation.

Cool batch to 0±5° C.

Charge 3509 g (16.59 mol, 1.2 equiv) of potassium phosphate to the 60 L reactor.

Charge 4.98 kg (1.5 V) of DMAc to the 60 L reactor.

Degas/inert the 60 L reactor (3 cycles of vacuum followed by nitrogen purge).

Heat the mixture to an internal temperature of 95±5° C. (reflux).

Hold the reaction at this temperature for 18 h ±6 h.

Adjust the batch to 20±5° C.

Cool to 0±5° C.

Charge 1688 g (15.21 mol, 1.1 equivalents) of 2-mercaptopyridine to the 60 L reactor.

Charge 3513 g (16.59 mol, 1.2 equivalents) of potassium phosphate to the 60 L reactor.

Degas/inert the 60 L reactor (3 cycles of vacuum followed by nitrogen purge).

Heat the mixture back up to an internal temperature of 95±5° C. (reflux).

Hold the reaction at this temperature for ≧6 hours.

Prepare a solution of 14.3 g of BHT to the distillate receiver.

Batch concentrate to reduce the reaction volumes by 12.3 L (˜3.5V) while maintaining the temperature <100° C. (use lower pressure when necessary).

Adjust the batch down to 50±5° C.

Charge 19.3 kg of water (5.5V) to the 60 L reactor.

Adjust the batch to 50±5° C.

Agitate for ≧15 min.

Stop agitation and allow layers to separate for ≧5 min.

Remove the bottom aqueous layer and discard into waste carboys.

Charge 10.4 kg of water (3.0 V) to the 60 L reactor.

Charge 11.7 kg of toluene (3.85V) to the 60 L reactor.

Charge 4.72 kg of 2-methylpropanol 1.65V) to the 60 L reactor.

Initiate agitation.

Adjust the batch to 50±5° C. internal temperature.

Agitate for ≧15 min.

Stop agitation and allow layers to separate for ≧5 min.

Remove the bottom aqueous layer from the 60 L reactor and transfer it to clean carboys.

Transfer the upper organic layer from the 60 L reactor to the 50 L receiver.

Transfer the aqueous layers from the carboys into the 60 L reactor.

Charge 11.7 kg of toluene (3.85V) to the 60 L reactor.

Charge 4.60 kg of 2-methylpropanol (1.65 V) to the 60 L reactor.

Initiate agitation.

Adjust the batch to 50±5° C. internal temperature.

Agitate for ≧15 min.

Stop agitation and allow layers to separate for ≧5 min.

Remove the bottom aqueous layer and transfer it to waste carboys.

Transfer the organic layer from the 60 L reactor to the 50 L receiver.

Clean the 60 L reactor by rinsing with water.

Transfer the organic mixture from the 50 L receiver to the 60 L through an inline 5 μm cartridge filter.

Charge 1748 mL of toluene to the 50 L receiver

Transfer rinse from the 50 L receiver to the 60 L through an inline 5 μm cartridge filter.

Batch concentrate the contents of the 60 L reactor to reduce the reaction volumes by 33 L (8.5-9V) while maintaining the temperature <100° C. (use lower pressure). The usual product concentration at this point is 220 mg/mL.

Adjust the batch to 40±5° C.

Prepare a slurry of seed in an appropriate container: 34.9 g 3-(2-methylpyridin-3-yloxy)-5-(pyridin-2-ylthio)pyridine 1-oxide (2648716) in 70 mL toluene+70 mL heptane.

Charge the seed slurry to the 60 L reactor.

Rinse the seed container with 70 mL toluene+70 mL heptanes and charge to the 60 L reactor.

Age 1 h at 40±5° C.

Add 10.74 kg of heptane (4.5V) over 2±1 h.

Cool to 20±5° C.

Agitate for ≧6 h.

Filter 60 L reactor contents using an Aurora filter equipped with a 12 μm PTFE cloth.

Charge 3.5 L of Heptane to an appropriate container.

Charge 3.5 L of Toluene to the same container.

Transfer container contents into the 60 L reactor as a rinse

Wash the wetcake with the contents of the 60 L reactor.

Charge 4.8 L of Heptane to an appropriate container.

Wash the wetcake with the contents of the 60 L reactor.

Dry product cake on filter under nitrogen stream at ambient temperature until LOD is ≦1.0% by TGA.

A total of 2.583 kg of 3-(2-methylpyridin-3-yloxy)-5-(pyridin-2-ylthio)pyridine 1-oxide was isolated. The product was found to be 97.8 wt %. The adjusted isolated yield=57.9%. The total yield of the reaction was 68.8%.

Analytical Data:

mp: 130° C. (DSC); ¹H NMR (CDCl₃, 400 MHz) δ 8.46 (dd, J=4.9 Hz, J=1.0 Hz, 1H), 8.42 (dd, J=4.7 Hz, J=1.2 Hz, 1H), 8.07 (dd, J=1.4 Hz, J=1.4 Hz, 1H), 7.85 (dd, J=1.8 Hz, J=1.8 Hz, 1H), 7.63 (ddd, J=7.8 Hz, J=7.8 Hz, J=2.0 Hz, 1H), 7.33 (dd, J=8.2 Hz, J=1.2 Hz, 1H), 7.30 (d, J=7.8 Hz, 1H), 7.23-7.17 (m, 2H), 6.94 (dd, J=1.7 Hz, J=1.7 Hz, 1H), 2.48 (s, 3H); ¹³C (CDCl₃, 100 MHz) δ 155.49, 155.00, 151.65, 150.42, 148.59, 146.59, 137.41, 136.45, 133.53, 129.11, 127.74, 124.40, 122.53, 122.20, 118.09, 19.33.

### Example 5

### Preparation of 2-chloro-3-((2-methylpyridin-3-yl)oxy)-5-(pyridin-2-ylthio)pyridine p-toluenesulfonate

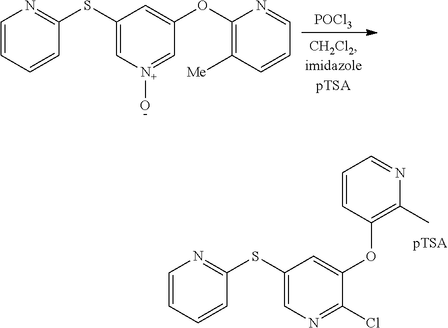

| | | | | Vol. | | | | |
|---|---|---|---|---|---|---|---|---|
| Step | Material | Wt % | Eq. | (relative to SM) | Mass | | Vol | Mol |
| 1 | 5-(2-methylpyridin- | 96.9 | 1 | — | 1.0 | kg | — | 3.21M |

| | 3-yloxy)-3-(pyridin- | | | | | | | |
|---|---|---|---|---|---|---|---|---|
| | 2-ylthio)pyridine 1- | | | | | | | |
| | oxide (2648716) | | | | | | | |
| 2 | Dichloromethane | — | — | 8.0 V | — | 8.0 | L | — |

| | | | | | | | | |
|---|---|---|---|---|---|---|---|---|
| 4 | Imidazole | — | 1.5 | — | 327.8 | g | — | 4.82 |

| 6 | Dichloromethane | — | — | 2.0 V | — | 2.0 | L | — |
|---|---|---|---|---|---|---|---|---|
| 10 | Phosphorus | — | 1.5 | — | — | 449 | mL | 4.82 |
| | Oxychloride | | | | | | | |
| 14 | Methanol | — | 7.7 | 1.0 V | | 1.0 | L | |

| | | | | | | | |
|---|---|---|---|---|---|---|---|
| 17 | Potassium Phosphate | — | 3.11 | — | | — | |
| | Dibasic | | | | | | |
| 18 | Water | — | — | 10.0 V | | | — |
| 20 | 1.0M K₂HPO₄ solution | — | — | As Needed | — | — | — |
| | (aqueous) | | | | | | |
| 25 | Water | — | — | 3.0 V | | | — |
| 30 | Water | — | — | As Needed | — | — | — |
| 31 | 2-Propanol (IPA) | — | — | As Needed | — | — | — |
| 35 | 2-Propanol (IPA) | — | — | 3.0 V | | | — |
| 36 | 6-chloro-5-(2- | — | — | — | | — | |
| | methylpyridin-3- | | | | | | |
| | yloxy)-3-(pyridin- | | | | | | |
| | 2-ylthio)pyridine | | | | | | |
| | (2679823) | | | | | | |
| 38 | Water | — | — | As Needed | — | — | — |
| 40 | p-Toluenesulfonic | — | 1.1 | — | | — | |
| | Acid Monohydrate | | | | | | |
| | (pTSA) | | | | | | |
| 41 | 2-Propanol | — | — | 2.0 V | | | — |
| | [IPA] | | | | | | |
| 43 | p-TSA/IPA solution | — | — | 0.2-0.4 v | | | — |
| 44 | Seed 6-chloro-5-(2- | — | 1% wt | — | | — | |
| | methylpyridin-3- | | | | | | |
| | yloxy)-3-(pyridin-2- | | | | | | |
| | ylthio)pyridinium 4- | | | | | | |
| | methylbenzenesulfonate | | | | | | |
| 45 | 2-Propanol (IPA) | — | — | As Needed | — | — | — |
| 48 | p-TSA/IPA solution | — | — | 1.6-1.8 v | | | — |
| 55 | 2-Propanol (IPA) | — | — | 2.0 V | | | — |
| 56 | 2-Propanol (IPA) | — | — | 2.0 V | | | — |

1. Charge 5-(2-methylpyridin-3-yloxy)-3-(pyridine-2-ylthio)pyridine-1-oxide (Example 4, 1.0 kg, 3.21 mol) to reactor I.

2. Charge Dichloromethane (8.0 L, 8 v) to the reactor I.

3. Agitate reactor I contents until dissolution is achieved.

4. Charge Imidazole (327.8 g, 1.5 eq.) to Reactor II.

5. Transfer container contents through an inline filter (0.45 μm) into the Reactor II.

6. Charge Dichloromethane (2.0 L, 2 v) to the container as a rinse.

7. Transfer rinse from container through the same filter into Reactor II.

8. Initiate agitation and N₂ sweep in the Reactor II (a NaOH SCRUBBER is set up).

9. Adjust batch temperature in reactor II to 10±5° C.

10. Charge Phosphorus Oxychloride (449 mL, 1.5 eq.) to the Reactor II maintaining a batch temperature of 10±5° C. Charge is exothermic.

11. Agitate batch in reactor II for ≧16 hours at 10±5° C.

12. Sample batch in reactor II and record details in the Sample Log.

13. Adjust the batch at 25±5° C.

14. Charge Methanol (1.0 L, 1 v) to the Reactor maintaining a batch temperature of 25±5° C. Charge is exothermic.

15. Agitate batch in reactor II for ≧4 hours at 25±5° C.

16. Sample batch in reactor II and record details in the Sample Log.

17. Charge Potassium Phosphate Dibasic (541.7 g, 3.11 eq.) to a carboy.

18. Charge Water (10 L) to the same carboy (1.0M Potassium Phosphate Dibasic solution).

19. Agitate carboy contents until dissolution is achieved.

20. Charge Potassium Phosphate Dibasic solution to the Reactor II to adjust pH maintaining a batch temperature of 25±5° C.

21. Stop agitation in reactor and allow layers to separate for ≧5 mins.

22. Drain the bottom organic layer.

23. Drain the upper aqueous layer.

24. Charge the organic layer back to the Reactor II.

25. Charge Water (3 v) to the Reactor II.

26. Agitate batch in reactor II for ≧5 mins.

27. Stop agitation in reactor II and allow layers to separate for ≧5 mins.

28. Drain the bottom organic layer.

29. Drain the upper aqueous layer.

30. Clean the Reactor II with Water/IPA (1:1).

31. Clean the Reactor II with 2-Propanol.

32. Transfer organic layer back to the Reactor through an inline filter (5 micron)

33. Adjust the batch to 30±5° C.

34. Concentrate the batch by removing 7.0±0.5 V maintaining a batch temperature of 30±5° C.

35. Charge 2-Propanol (3 v) to the Reactor II.

36. Concentrate the batch by removing 3.0±1.0 V maintaining a batch temperature of 30±5° C.

37. Transfer the batch to the crystallization reactor.

38. Charge Water to the Reactor to reach a batch concentration of 1.0±0.5 wt % H₂O.

39. Adjust batch to 50±5° C.

40. Charge p-Toluenesulfonic Acid Monohydrate (1.1 eq. against free base) to an appropriate container.

41. Charge IPA to the same container.

42. Agitate container contents until dissolution is achieved.

43. Charge a portion of the p-TSA/IPA (10±5% v/v) solution to the Reactor maintaining a batch temperature of 50±5° C.

44. Charge Seed (1% wt) to an appropriate container.

45. Charge IPA to the same seed container.

46. Charge Seed/IPA slurry to the Reactor.

47. Agitate batch at 50±5° C. for ≧30 min.

48. Charge the remainder of the p-TSA/IPA solution to the Reactor maintaining a batch temperature of 50±5° C.

49. Agitate batch for ≧1 hr.

50. Adjust batch temperature to 20±5° C. over 6±1 hr.

51. Agitate batch for ≧10 hrs at 20±5° C.

52. Filter the batch using an Aurora filter equipped with a 12 μm PTFE filter cloth.

53. Charge the mother liquor back to the reactor II as a rinse.

54. Wash the cake with recycled mother liquor.

55. Wash the cake with IPA.

56. Wash the cake with IPA.

57. Dry wet cake on the filter under N₂/vacuum at ambient temperature until LOD is ≦2.0% by TGA.

Analytical Data:

¹H NMR (DMSO, 400 MHz): δ 8.55 (m, 1H), 8.45 (bt, 1H), 8.42 (bd, 1H), 7.88 (m, 2H), 7.74-7.68 (m, 2H), 7.51 (m, 2H), 7.31 (m, 1H), 7.26 (m, 1H), 7.14 (m, 2H), 2.62 (m, 3H), 2.31 (s, 3H) ppm; ¹³C (DMSO, 100 MHz): 156.81, 151.62, 149.71, 149.21, 147.48, 147.41, 145.38, 141.97, 138.91, 137.86, 137.81, 134.1, 131.26, 128.86, 128.11, 125.49, 125.25, 122.27, 121.51, 20.79, 15.92.

### Example 6

### Synthesis of (S)-3-(1,4-dioxaspiro[4.5]decan-2-yl)-1,2,4-thiadiazol-5-amine

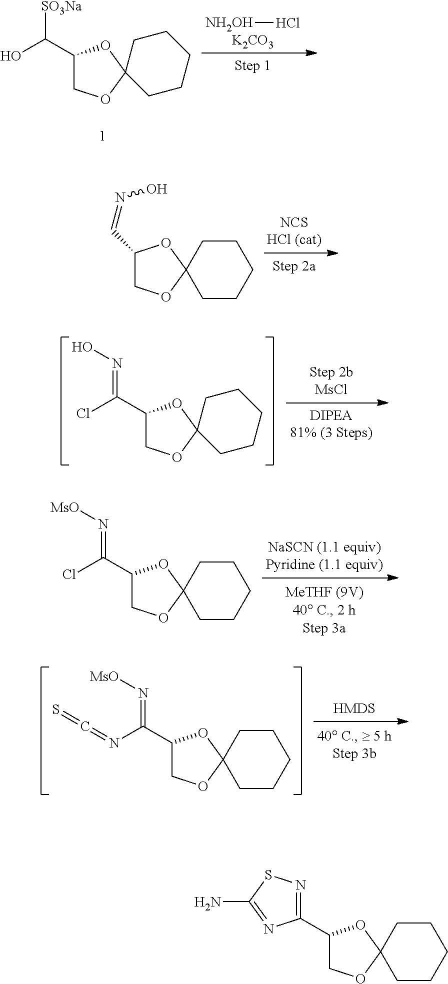

| Materials | CAS# | Supplier |
|---|---|---|
| sodium hydroxy((R)-1,4- | N/A | AMRI |
| dioxaspiro[4.5]decan-2- | | |
| yl)methanesulfonate (1) | | |
| Hydroxyamine hydrochloride | 5470-11-1 | Sigma Aldrich |
| Potassium carbonate | 584-08-7 | Sigma Aldrich |
| Water | 7732-18-5 | AMA DI |
| 2-Methyltetrahydrofuran (2-MeTHF) | 96-47-9 | Alfa Aesar |

| Material | Grams | mL | Mol | Equiv |
|---|---|---|---|---|
| sodium hydroxy((R)-1,4- | 10000 | — | 36.46 | 1.00 |
| dioxaspiro[4.5]decan-2- | | | | |
| yl)methanesulfonate (1) | | | | |
| Hydroxyamine hydrochloride | 2914 | — | 41.93 | 1.15 |
| Potassium carbonate | 6047 | — | 43.75 | 1.20 |
| Water | 16300 | 16300 | — | — |
| Water | 1800 | 1800 | — | — |
| 2-Methyltetrahydrofuran (2-MeTHF) | 34120 | 40000 | — | — |
| 2-Methyltetrahydrofuran (2-MeTHF) | | | — | |
| 2-Methyltetrahydrofuran (2-MeTHF) | 853 | 1000 | — | — |

Step 1: To a coiled 100-L reactor was charged with solid sodium hydroxy((R)-1,4-dioxaspiro[4.5]decan-2-yl)methanesulfonate (10,000 g, 36.46 mol, 1.0 equivalent), followed by 2-MeTHF (40000 mL, 4V) to the reactor and the suspension was agitated. To a 60-L jacketed reactor was charged water (16300 g, 1.63V), followed by potassium carbonate (6047 g, 43.75 mol, 1.2 equivalents), and agitated to complete dissolution of potassium carbonate. To the potassium carbonate solution in the 60-L at <25° C. was charged hydroxyamine hydrochloride (2914 g, 41.93 mol, 1.15 equivalents) in 5 portions, maintaining the batch temperature at 25±5° C. to give a homogenous hydroxyamine aqueous solution. To the 100-L was charged the hydroxyamine solution in the 60-L over no less than 30 min. Water (1800 mL, 0.18V) was charged to rinse the 60-L, and charged into the 100-L reactor. After completion of charge, the thick heterogeneous mixture in the 100-L reactor was vigorously agitated until the mixture became a clear aqueous and organic biphasic, which typically takes 3-5 h. Upon completion of the reaction, the bottom aqueous layer was discarded and the top organic stream was distilled in a 60-L reactor under reduced pressure (ca. 150 torr) at 40° C., and then azeotropically dried by distillation with 2-MeTHF (10000 mL) The oxime product solution was collected, and the 60-L reactor was rinsed with 2-MeTHF (1000 mL), combined with the oxime product solution to give a colorless solution (12.27 kg). The resulting oxime was used without further purification. ¹H NMR (with added benzyl benzoate internal standard CDCl₃, 400 MHz) δ 9.15 (0.37H, br), 8.99 (0.63H, br), 6.93 (0.37H, d, J=4.2 Hz), 5.12 (0.37H, td, A=6.9 Hz, J_{d}=4.2 Hz), 4.65 (0.63H, ddd, J=6.9, 6.5, 6.4 Hz), 4.35 (0.3711, dd, J=8.4, 7.0 Hz), 4.14 (0.64H, dd, J=8.5, 6.6 Hz).

### Steps 2a and 2b: Preparation of (R, Z)-2-(chloro(methylsulfonyloxyimino)methyl)-1,4-dioxaspiro[4.5]decane

| Material | Grams | mL | Mol | Equiv |
|---|---|---|---|---|
| (S)-1,4-dioxaspiro[4.5]decane- | 6000 | — | 32.39 | 1.00 |
| 2-carbaldehyde oxime | | | | |
| 2-Methyltetrahydrofuran (2-MeTHF) | 23000 | 27000 | — | — |
| N,N-dimethylacetamide (DMAc) | 2900 | 3090 | — | — |
| Hydrogen chloride solution | — | 160 | 0.65 | 0.02 |
| (4.0M in dioxane) | | | | |
| N-chlorosuccinimide | 4541 | — | 34.01 | 1.05 |
| Methanesulfonyl chloride | 3896 | 2632 | 34.01 | 1.05 |
| N,N-diisopropylethylamine | 4602 | 6200 | 35.63 | 1.10 |
| water | 12000 | 12000 | — | — |
| 15 w/w % sodium chloride | | 12000 | — | |
| 15 w/w % sodium chloride | | 12000 | — | |
| 15 w/w % sodium chloride | | 6000 | — | |
| 15 w/w % sodium chloride | | 6000 | — | |
| 25 w/w % sodium chloride | | 6000 | — | |
| 2-Methyltetrahydrofuran (2-MeTHF) | | 600 | — | |
| heptane | | 81200 | — | — |
| 20:1 heptane/2-Methyltetra- | — | 12000 | — | — |
| hydrofuran (v/v) | | | | |

To a 60 L jacket reactor was charged (S)-1,4-dioxaspiro[4.5]decane-2-carbaldehyde oxime (11280 g, 53.2 wt %, 32.39 mol, 1.00 equivalent) solution in 2-MeTHF through a ≦10 μm inline filter. To the 60-L was charged 2-MeTHF (20800 mL) to adjust total 2-MeTHF to be 4.5V, followed by addition of DMAc (2900 g, 0.5 V). The reaction mixture was agitated, and cooled to <15° C. (12° C.). To the reaction mixture in 60-L was charged 4M hydrogen chloride solution in dioxane (160 mL, 0.65 mol, 0.02 eq equivalents) at 11° C. To the 60-L was charged N-chlorosuccinimide (4541 g, 34.01 mol, 1.05 equivalents) in 10 portions while maintaining the reaction mixture at 10-20° C. Caution: Exothermic. After completion of NCS charge, the reaction mixture was warmed to 20±5° C. and aged for ≧15 min. The content in the 60-L was cooled to 0±5° C. (1.4° C.) and methanesulfonyl chloride (3896 g, 2632 mL, 34.01 mol, 1.05 equivalents) was charged. To the reaction mixture in the 60-L was charged N,N-diisopropylethylamine (4602 g, 6200 mL, 35.63 mol, 1.10 equivalents) over no less than 1 hour while controlling the rate of charge so that the batch temperature ≦10° C. (Jacket temperature set −20° C., Tₘₐₓ=4.4° C.). The reaction heterogeneous mixture was age at 0±10° C. for no less than 30 min. The content in the 60-L was warmed to 15° C., and water (12000 g, 2V) was charged, and agitated for no less than 2 min. The bottom aqueous layer was discarded and the top organic layer was washed with 15% sodium chloride solution (12000 mL) twice, 15% sodium chloride (6000 mL, 1V) twice, 25% sodium chloride solution (6000 mL, 1V). The resulting crude organic stream was distilled in the 60-L under reduced pressure at <30° C. until about 15 L, and then azeotropically dried by distillation with 2-MeTHF under reduced pressure at <30° C. The crude product stream was collected and the 60-L was rinsed with 2-MeTHF (1230 mL) and combined to give the crude product. The crude product solution was filtered through a ≦10 μm inline filter into a clean 100-L coiled reactor, and rinsed with 2-MeTHF (500 mL) to bring the total 2-MeTHF to 14.44 L, based on which, total heptane used in the crystallization would be 81.2 L to a final of 15/85 (v/v) 2-MeTHF/heptane. To the crude solution in the 100-L in 2-MeTHF was charged heptane (10 L) at 20° C. over 10 min, and then seeded with (90.5 g) slurry in heptane (750 mL), and aged for no less than 5 min. Additional heptane (70500 mL) was charged over 1 h to bring the final total volume of heptane to 81.3 L. After completion of heptane charge, the slurry was aged at 20° C. for 15 min. The slurry in the 100-L was cooled to 0° C. and aged for another 30 min. The slurry was filtered with Aurora filter fitted with a 25 μm polypropylene filter cloth. To the 100-L was charged a mixed solvent of 95/5 (v/v) heptanes/2-MeTHF (12000 L, 2V) to rinse the reactor and charged into the Aurora filter to wash the cake. The filter cake was dried on frit under nitrogen stream at ambient temperature until organic solvent ≦1% by TGA analysis to give 8246 g of chlorooxime mesyalte. A white crystalline solid in 84.5% yield, 81% yield from 1. mp: 67-69° C.; ¹H NMR (CDCl₃, 400 MHz) δ 4.94 (1H, dd, J=6.8, 5.1 Hz), 4.29 (1H, dd, J=9.0, 6.8 Hz), 4.19 (1H, dd, J=9.1, 51 Hz), 1.81-1.77 (2H, m), 1.71-1.60 (6H, m), 1.48-1.42 (2H, m).

### Steps 3a and 3b: Preparation of (S)-3-(1,4-dioxaspiro[4.5]decan-2-yl)-1,2,4-thiadiazol-5-amine

Stoichiometry table*

| Material | Equiv. | Volume | Mass | Volume | Moles |
|---|---|---|---|---|---|
| Chloromesylate | 1.00 | | 4,000 g | | 13.4 |
| ((R,Z)-2- | | | | | |
| (chloro(methylsul- | | | | | |
| fonyloxyimino)methyl)-1,4- | | | | | |
| dioxaspiro[4.5]decane) | | | | | |
| Sodium Thiocyanate (NaSCN) | 1.1 | — | 1,194 g | — | 14.74 |
| 2Me-THF | — | 9.0 | 30,960 g | 36,000 mL | — |
| Pyridine | 1.05 | | l,116 g | l,150 mL | 14.1 |
| Hexamethyldisilazane | 1.1 | | 2,385 g | 3,081 mL | 14.8 |
| Water | | 2.0 | 8,000 g | 8,000 mL | |
| NaHSO₄ | 0.90 | | 1440 g | | 12.00 |
| Water | | 1.0 | 4,000 g | 4,000 mL | |
| Water | | 3.0 | 12,000 g | 12,000 mL | |
| Sodium Chloride (NaCl) | 1.98 | — | 2250 g | — | 38.5 |
| Sodium Bicarbonate | 1.33 | | 1,500 g | | 17.85 |
| Heptane | | 2.5 | 6,840 g | 10,000 mL | |
| Heptane | | 5.0 | 13,680 g | 20,000 mL | |
| (S)-3-(1,4-dioxaspiro[4.5]decan- | 2.5-5 wt % | | 100 g | | |
| 2-yl)-1,2,4-thiadiazol-5-amine | | | | | |
| 2Me-THF | — | 0.2 | 688 g | 800 mL | — |
| Heptane | | 1.8 | 4,924 g | 7,200 mL | |

*Unless otherwise noted in the procedure, equivalents and volumes are reported in reference to moles or mass of Chloromesylate.

Set the jacket temperature of a clean dry 60 L reactor (reactor 1) to 20±5° C. Charge Sodium Thiocyanate (1,195 g, 1.1 equivalents) to the 60 L reactor (reactor 1). Charge 2-MeTHF (28,000 mL, 24,080 g, 7.0 V) to the 60 L reactor (reactor 1). Initiate agitation in the 60 L reactor (reactor 1). Charge Pyridine (1, 116 mL, 1,150 g) to reactor 1. Stir the batch for no less than 0.25 h. Adjust the batch temperature to 40±5° C. Set the jacket temperature of a clean dry 15 L reactor (reactor 2) to 20±5° C. Charge Chloromesylate (4,000 g, 13.4 moles) to the 15 L reactor (reactor 2). Charge 2-MeTHF (8,000 mL, 6,880 g, 2.0 V) to the 15 L reactor (reactor 2). Initiate agitation in the 15 L reactor (reactor 2). Stir the batch no less than 0.25 h, until completely dissolved. Transfer the chloromesylate solution from the 15 L (reactor 2) to the 60 L (reactor 1) over approximately 1 h maintaining a batch temperature of 40° C. ±5° C. This addition is exothermic. Age the batch at 40° C. ±5° C. for no less than 1 h. Charge hexamethyldisilazane (2,385 g, 3,081 mL, 1.1 equivalents) to the 4 L addition funnel attached to the 60 L (reactor 1). Add hexamethyldisilazane from the 4 L addition funnel over approximately 1 h while maintaining an internal batch temperature at 40° C.±5° C. This addition is exothermic. Age the batch in the 60 L (reactor 1) at 40° C.±5° C. until Acylthiocyanate is consumed. Cool the batch in the 60 L reactor (reactor 1) to 5±10° C. Set the coil temperature of a clean dry 100 L reactor (reactor 3) to 20±5° C. Charge Water (8,000 mL, 8,000 g, 2V) to the 100 L reactor (reactor 3). Initiate agitation in the 100 L reactor (reactor 3) and set the coil temperature to 5±10° C. Transfer the batch from the 60 L (reactor 1) into the 100 L (reactor 3) over approximately 1 h maintaining internal temperature in reactor 3 below 10° C. This addition is exothermic. Adjust the internal temperature in the 100 L reactor (reactor 3) to 20±5° C. Prepare an aqueous NaHSO₄ solution by slowly adding NaHSO₄ (1,440 g, 0.90 equivalents) to Water (4,000 mL, 4,000 g, 1V) in an appropriate container. Charge the NaHSO₄ solution to the 100 L reactor (reactor 3). Agitate the 100 L reactor (reactor 3) for approximately 0.5±0.5 h. Allow the batch in reactor 3 settle for no less than 2 minutes. Isolate the (lower) aqueous layer. Prepare an aqueous solution by adding Sodium Bicarbonate (1,500 g, 1.33 equivalents) and Sodium Chloride (2,250 g, 2.87 equivalents) to Water (12,000 mL, 3V) in an appropriate container. Charge the Sodium Bicarbonate/Sodium Chloride Solution (12000 mL, 3V) to the 100 L reactor (reactor 3). Agitate the 100 L reactor (reactor 3) for 0.5±0.5 h. Allow the batch in the 100 L reactor (reactor 3) to settle for no less than 2 minutes. Isolate the (lower) aqueous layer in an appropriate container. Rinse the 60 L reactor (reactor 1) with Water and Acetone or Methanol. Rinse the 60 L reactor (reactor 1) with 2 MeTHF. Set the jacket temperature of a clean, dry 60 L reactor (reactor 1) to 20±5° C. Transfer batch from the 100 L (reactor 3) to the 60 L (reactor 1). Concentrate batch in the 60 L reactor (reactor 3) from 9V to approximately 6V, while maintaining the internal temperature of the vessel below 55° C. Distill while adding 2 MeTHF to maintain constant volume (24,000 mL, 6V) while maintaining the internal temperature of the vessel below 55° C. by adjusting the vacuum pressure until ≦0.5% water is obtained. Cool the batch in the 60 L reactor (reactor 1) to 20±5° C. Rinse the 60 L reactor (reactor 1) with Water and Acetone or Methanol. Rinse the 60 L reactor (reactor 1) with 2 MeTHF. Insert a R55 S carbon cartridge to the CUNO filter with a 5 μm inline filter attached to the CUNO outlet and rinse filter for no less than 5 min with 2-Methyltetrahydrofuran. Filter the batch through the CUNO housing into the clean and dry 60 L reactor (reactor 1) at approximately 750 mL/min. Rinse the CUNO filter with no less than 2 L MeTHF, combine with batch into the 60 L reactor. Concentrate batch in the 60 L (reactor 1) to approximately 2.0V, while maintaining the internal temperature of the vessel below 55° C. by adjusting the vacuum pressure. Cool the batch in the 60 L reactor (reactor 1) to 20±5° C. Add 2-MeTHF to the 60 L reactor (reactor 1) to adjust 2-MeTHF to 2V. Add Heptane to the 60 L reactor (reactor 1) to achieve a 60:40 MeTHF:Heptane ratio. Add finely ground Seed (100 g, 2.5 wt %) as a slurry in a minimal volume of heptanes to the batch in the 60 L reactor (reactor 1). Add Heptane to the 60 L reactor (reactor 1) to achieve a 20:80 MeTHF:Heptane ratio over no less than 3 h. Filter the crystallized product through an Aurora filter fitted with a ≦25 μm filter cloth. Prepare a 10% 2-MeTHF: heptanes by adding 2 MeTHF (800 mL, 688 g) to heptane (7200 mL, 4,924 g) in an appropriate container. Wash reactor with two portions of 10% 2-MeTHF: Heptane (1V, 4 L). Dry the product cake on the Aurora filter under a nitrogen stream at RT for at least 1 h.

| Batch | Chemical | Isolated Yield | KF water | LCAP₂₀₀/ |
|---|---|---|---|---|
| (Scale) | Yield | (mass) | (ppm) Step 8 | LCAP₂₅₄ |
| (107406-3) | 90% | 75% | 1708 | 99.6/100 |
| 5.13 kg | | (3.20 kg) | | |
| (107406-4) | 91% | 76% | 163 | 100/100 |
| 2.80 kg | | (1.75 kg) | | |

(S)-3-(1,4-dioxaspiro[4.5]decan-2-yl)-1,2,4-thiadiazol-5-amine was isolated as a pale yellow agglomerated crystalline solid: MP (DSC)=117.1° C., 121.3° C.; ¹H NMR (CDCl₃, 400 MHz) δ 6.77 (br s, 2H), 5.05 (dd, J=6.1, 0.7 Hz, 1H), 4.28 (ddd, J=20.5, 6.4, 2.0 Hz, 2H), 1.75-1.31 (m, 10H); ¹³C NMR (CDCl₃, 100 MHz) δ 184.2, 170.9, 111.2, 73.9, 67.8, 35.7, 34.8, 25.0, 23.9, 23.8.

### Example 7

### N-(3-(2-methylpyridin-3-yloxy)-5-(pyridin-2-ylthio)-3-(1,4-dioxaspiro[4.5]decan-2-yl)-1,2,4-thiadiazol-5-amine

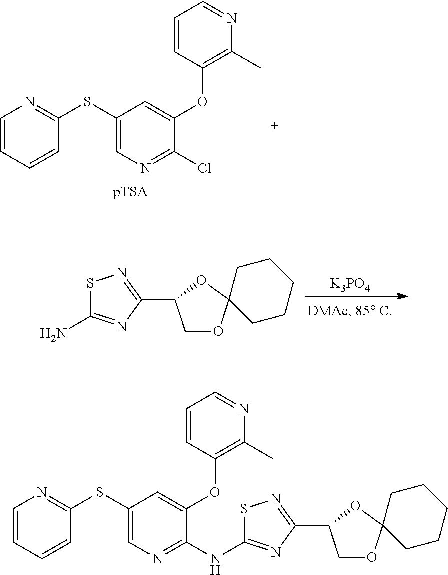

To a 1 L 3-necked RBF equipped with a condenser, a mechanical stirrer, and a temperature probe, under an atmosphere of N₂, was added 6-chloropyridine (Ex. 5 product, 50.0 g, 97.4 mmol), followed by c-Hex-5-ATDZ (Ex 6 product, 1.15 equivalents, 112.0 mmol, 27.03 g), potassium phosphate (2 equivalents, 194.8 mmol, 41.35 g) and dimethylacetamide (150 mL, 3 volumes). The flask was then degassed with stirring (3 cycles, vacuum <60 torr, backfill with Ar). The slurry was then heated to 85° C. with stirring. A second charge of potassium phosphate (2 eq, 194.8 mmol, 41.35 g) was added after 2 h and the slurry degassed as previously described. A third charge of potassium phosphate (2 eq, 194.8 mmol, 41.35 g) was added after 4 h and the slurry degassed as previously described. A fourth charge of potassium phosphate (2 eq, 194.8 mmol, 41.35 g) was added after 6 h and the slurry degassed as previously described. The slurry was stirred at 85° C. overnight. After 21 h at 85° C. complete conversion of the starting material was observed. The reaction mixture was cooled to RT. Water (250 mL, 5 volumes) was added slowly while maintaining T<40° C. Toluene (200 mL, 4 volumes) and 2-BuOH (50 mL, 1 volume) were then added. The pH was adjusted from 12 to 7.7 by the addition of 5N HCl (155 mL). The mixture was transferred to a separatory funnel and the phases were allowed to separate into 3 phases. Water (250 mL, 5 volumes) was added to the toluene phase in the separatory funnel. The mixture was vigorously shaken and the phases were allowed to separate. Brine (250 mL, 5 volumes) was added to the toluene phase in the separatory funnel. The mixture was vigorously shaken and the phases were allowed to separate. The toluene phase gave 44.4 g of the N-(3-(2-methylpyridin-3-yloxy)-5-(pyridin-2-ylthio)-3-(1,4-dioxaspiro[4.5]decan-2-yl)-1,2,4-thiadiazol-5-amine (85% solution assay yield). The solvent was then switched from toluene to EtOH. The EtOH solution was heated to 45° C. and seed (1 wt %, 444.5 mg) was added. The seed held after 30 minutes and the slurry was then allowed to cool to ambient temperature overnight. Water was then slowly added to the slurry (300 mL, 6 volumes). The supernatant concentration was 10.0 mg/g N-(3-(2-methylpyridin-3-yloxy)-5-(pyridin-2-ylthio)-3-(1,4-dioxaspiro[4.5]decan-2-yl)-1,2,4-thiadiazol-5-amine. The product was isolated by vacuum filtration and rinsed with a mixture of EtOH/water (1/1 v/v, 150 mL). The product was dried on the filter under a stream of nitrogen. N-(3-(2-methylpyridin-3-yloxy)-5-(pyridin-2-ylthio)-3-(1,4-dioxaspiro[4.5]decan-2-yl)-1,2,4-thiadiazol-5-amine was obtained as a tan solid (43.5 g, 99.6 PA %, 100 wt %, 83.5% potency adjusted yield, 6.5% loss to mother liquors, 1.7% loss to washes).

### Example 8

### Amorphous AMG151

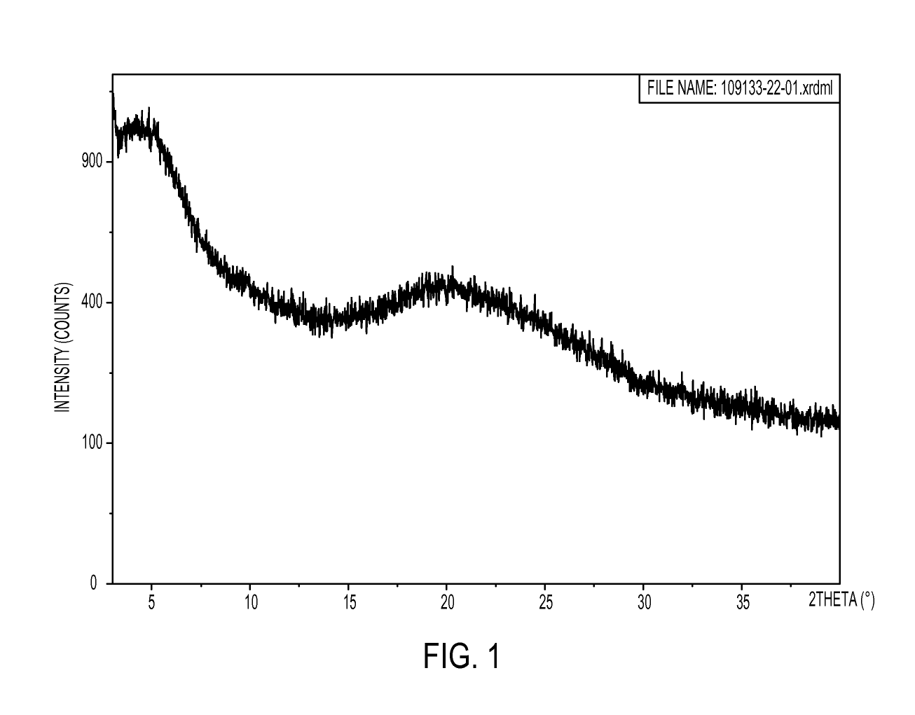

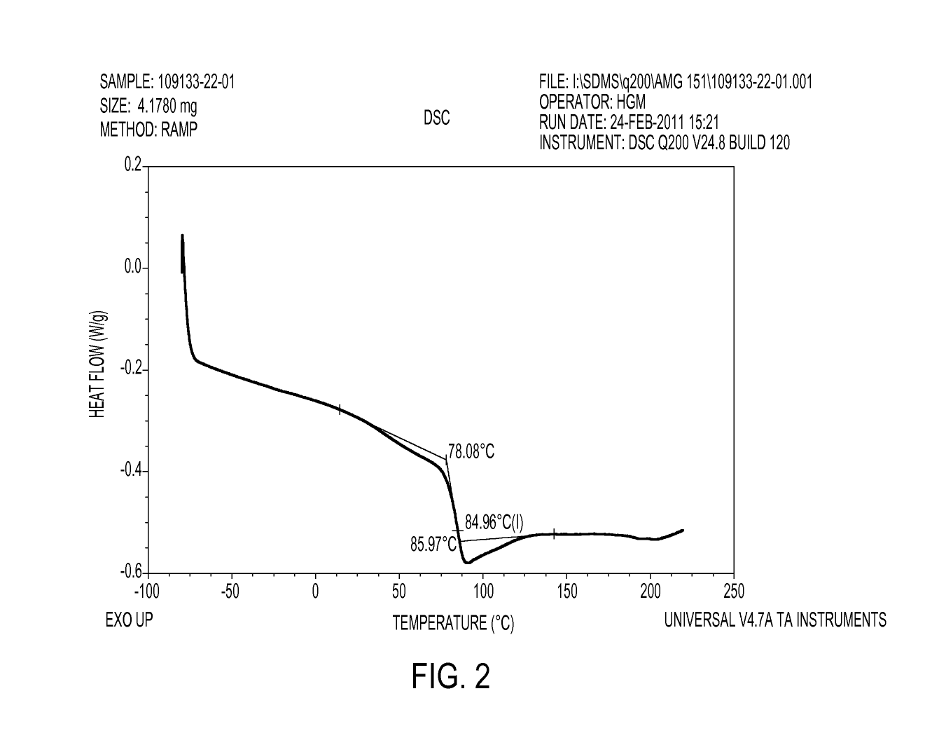

Amorphous AMG151 was generated via melt quenching and characterized. The data are shown in FIGS. 1-2. The sample was found to have a glass transition around 78° C. and to be hygroscopic via vapor adsorption (phase remained unchanged post experiment).

### Example 9

### Preparation of AMG151 DiHCl salt

1 g of 3-((2-methylpyridin-3-yl)oxy)-5-(pyridin-2-ylthio)pyridin-2-amine (3.13 mmol; 1 equiv.) was combined with 0.807 g (7.2 mmol, 2.3 equiv.) potassium t-butoxide in 10 ml of THF and the resulting slurry cooled to 0° C. In a separate flask, sulfone 3-[(4S)-2,2-dimethyl-1,3-dioxolan-4-yl]-5-[(4-methylphenyl)sulfonyl]-1,2,4-thiadiazole (1.278 g, 3.75 mmol, 1.2 equiv.) was dissolved in 2 ml THF and the resulting solution added dropwise to the reaction mixture. Quenched using 1 mL of water and neutralized with 0.24 mL AcOH. Removed aqueous layer and washed organic using 2 ml of 50% satd. Brine to provide (S)-3-(2,2-dimethyl-1,3-dioxolan-4-yl)-N-(3-((2-methylpyridin-3-yl)oxy)-5-(pyridin-2-ylthio)pyridin-2-yl)-1,2,4-thiadiazol-5-amine.

Deprotection/Isolation of DiHCl salt:

Added 3N HCl (2.5 equiv., 7.825 mmol, 2.6 ml) to the (S)-3-(2,2-dimethyl-1,3-dioxolan-4-yl)-N-(3-((2-methylpyridin-3-yl)oxy)-5-(pyridin-2-ylthio)pyridin-2-yl)-1,2,4-thiadiazol-5-amine. Added 25 mg as seed. Let stir overnight. Filtered the slurry and washed with 4 ml of THF. Dried on frit for 10 minutes before placing in a vacuum oven at 40° C. to provide the (S)-1-(5-((3-((2-methylpyridin-3-yl)oxy)-5-(pyridin-2-ylthio)pyridin-2-yl)amino)-1,2,4-thiadiazol-3-yL)ethane-1,2-diol[1.563 g (94.7% yield) calculated as DiHCl salt of AMG151].

### Example 10

### Analysis of AMG151 DiHCl salt

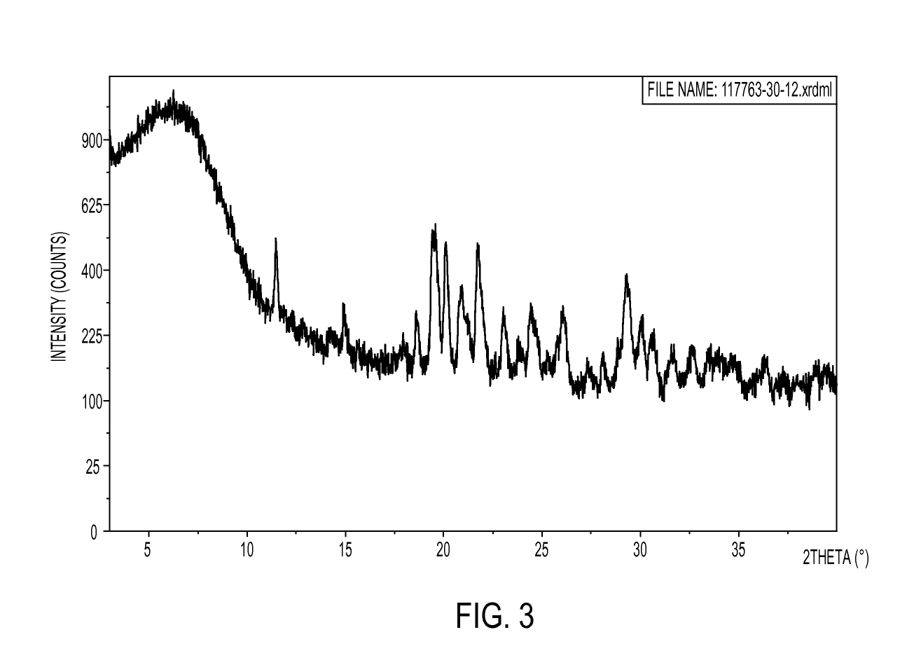

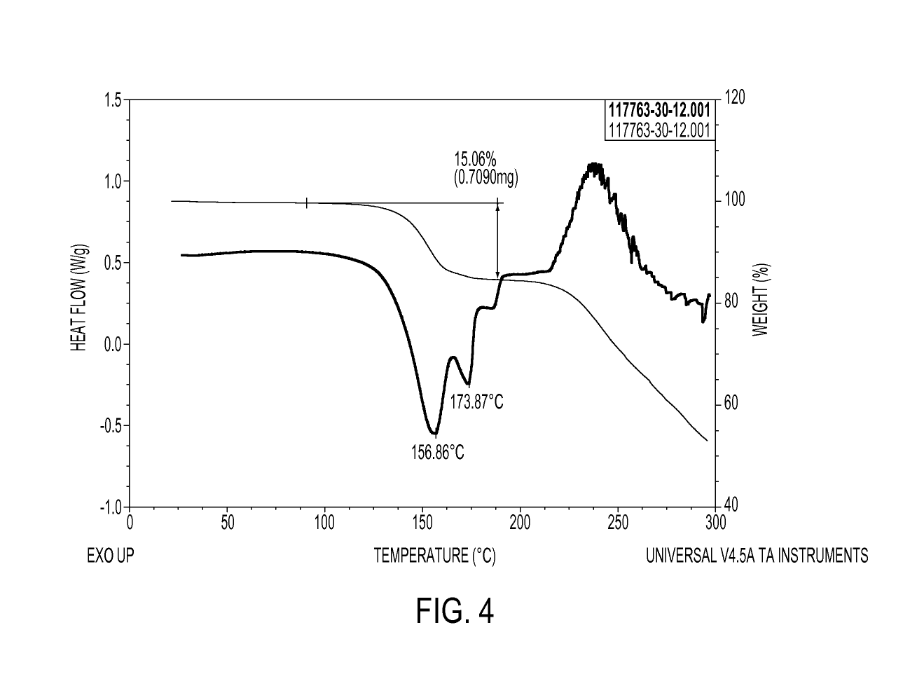

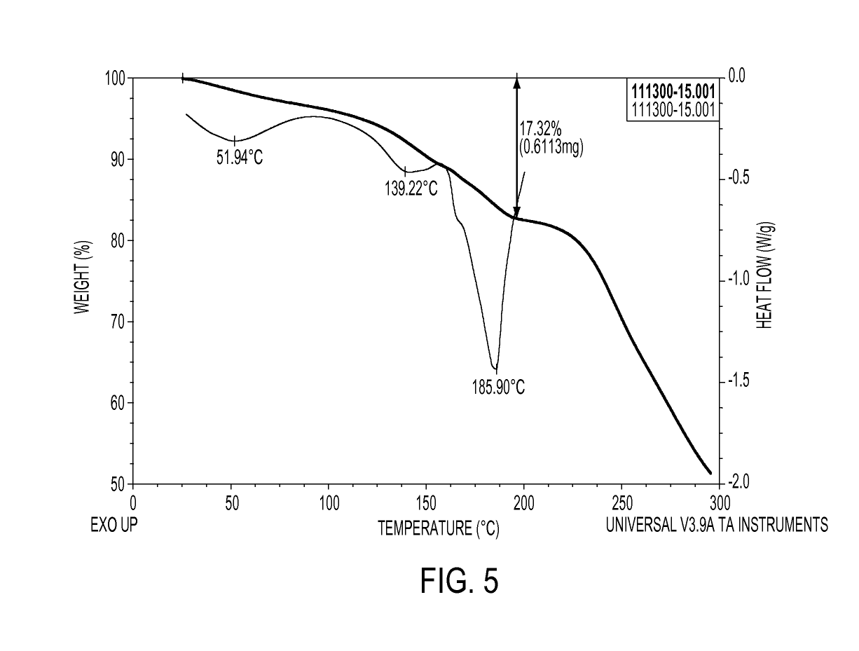

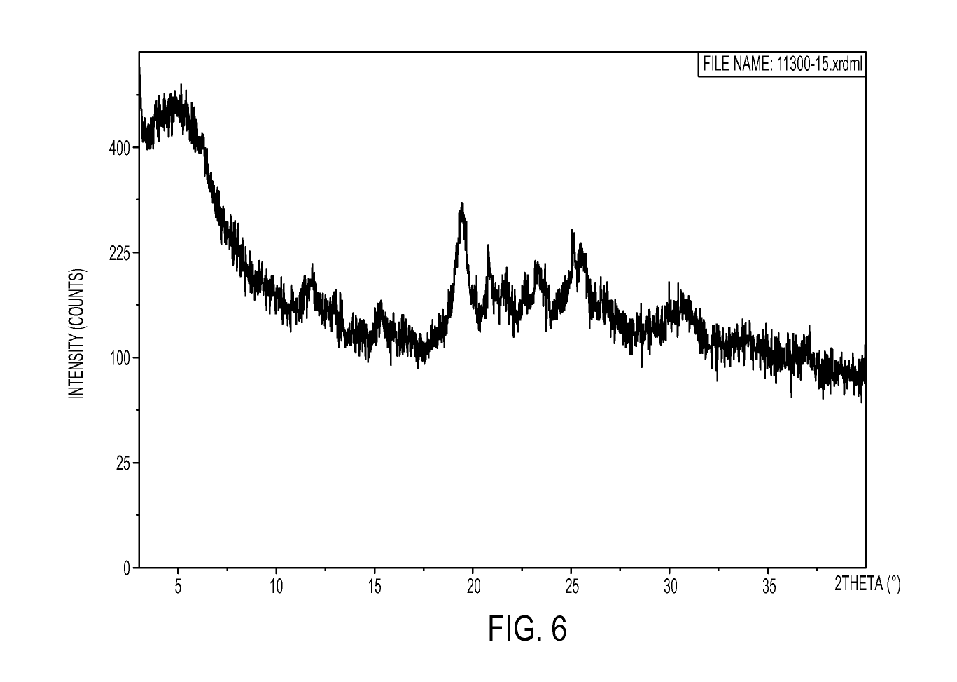

A representative XRPD pattern for Form B is shown in FIG. 3. The sample appears to be crystalline with an irregular morphology. This phase was further characterized via DSC and TGA and the data for Form B are shown in FIG. 4. The DSC curve indicates broad endotherms around 157 and 174° C. (attributed to desolvation and possibly a melt). The TG curve indicates a 15% weight loss up to 175° C. Data are shown in FIGS. 5-6 for Form A.

### Example 11

### Fumarate Solvate

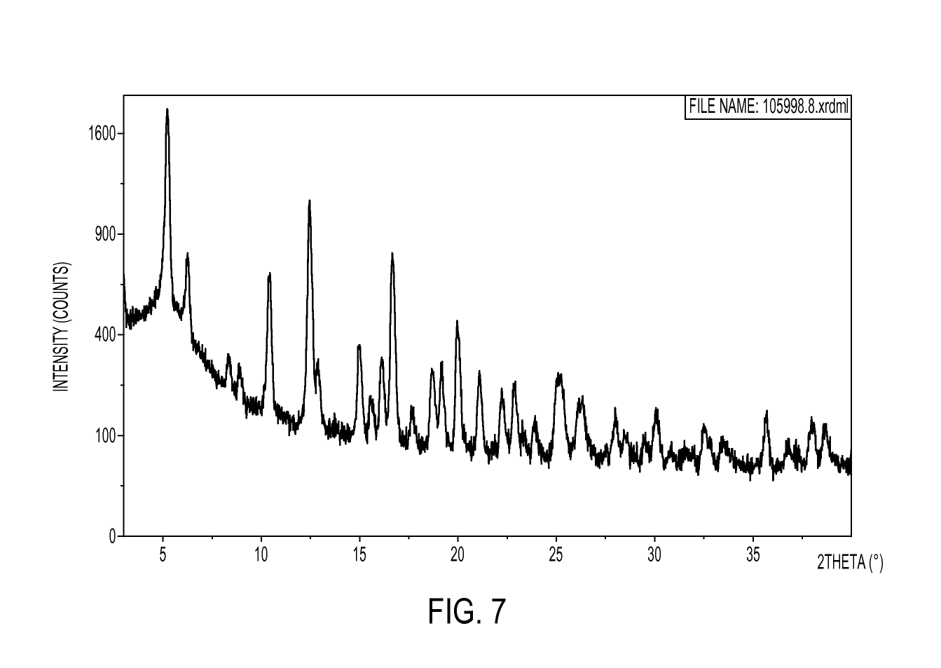

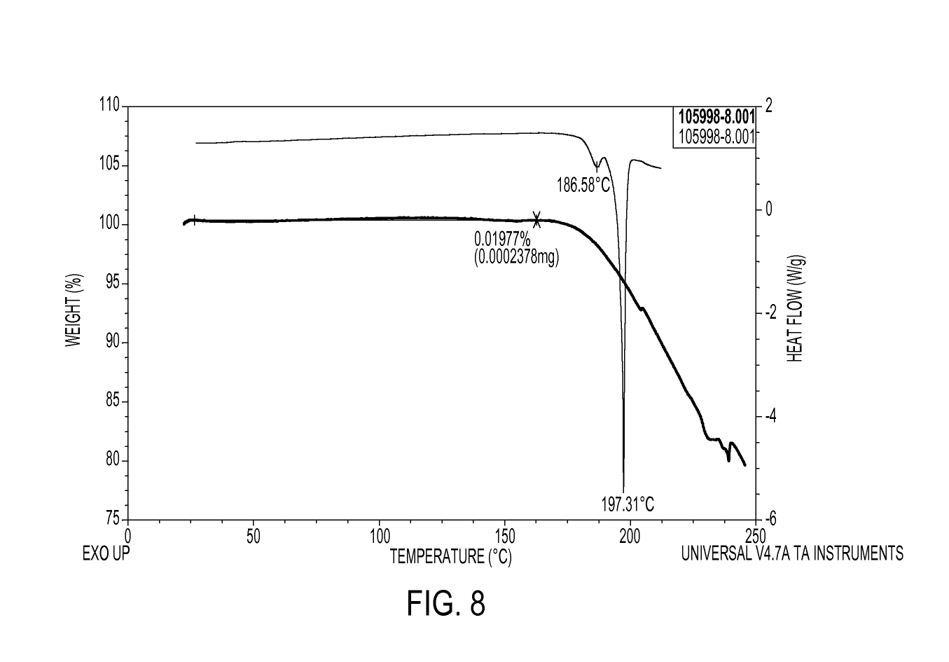

AMG151 freebase [7.5 g] and fumaric acid [2.3 g] (1.2 equivalents) was suspended in 75 mL of methanol and heated to reflux. An additional 30 mL of methanol and 5 mL of water was charged to the slurry and held at reflux for 2 hours. The reaction mixture was heat-cycled between ambient and reflux one time followed by cooling to room temperature. The slurry was filtered, washed with methanol and dried in air to obtain 7.5 g of the fumarte co-crystal of AMG151. The Fumarate solvate has a distinct XRPD pattern having unique reflections at around 5.2, 6.2, 10.4, 16.7, 19.9 degrees 2θ as shown in FIG. 7. TG data is shown in FIG. 8. Endotherms were observed at 187° C. and 197° C.

### Example 12

### Hydrate Form C Thermal Analysis

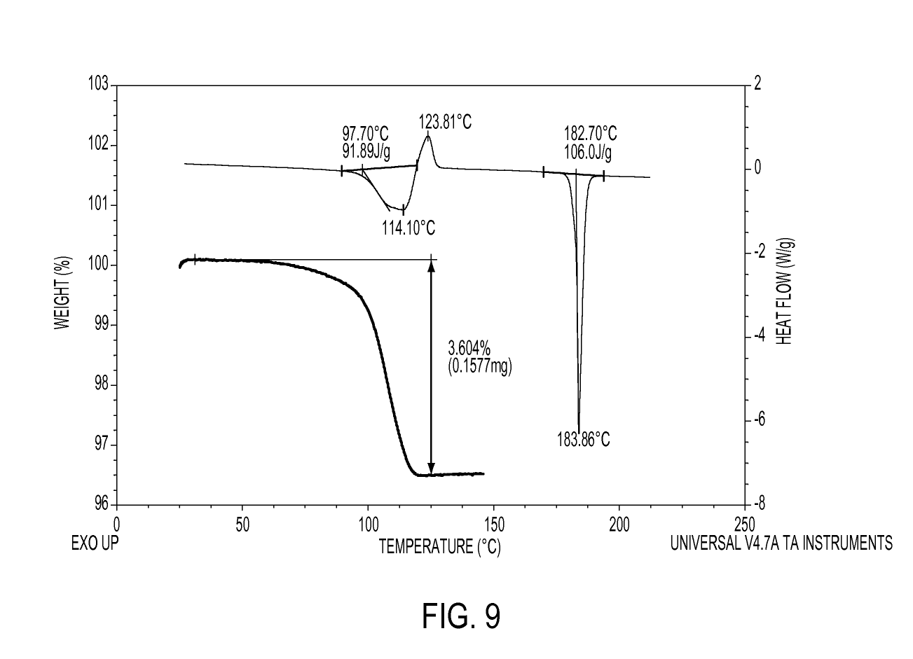

Thermal data is shown in FIG. 9. The DSC curve indicates an endothermic transition at 113° C. attributed to dehydration, an exotherm at 124° C. attributed to crystallization of a new form, and an endothermic transition [melt] at 183-184° C. attributed to a melt. AMG151. H₂O [Example 2] possesses a unit of water in the crystal matrix which begins to desolvate above 50° C. with complete desolvation above 100° C. The TGA curve shows a weight loss (3.6% up to 125° C.) indicating a solvate. Desolvation is followed by an exothermic event (recrystallization to form A). The hydrate form is a stoichiometric hydrate with a KF measured of 3.2-4.6% (3.9% theoretical) and weight loss is confirmed by TGA. Additionally, the hydrate is also non hygroscopic and shows <0.5% wt change from 5% to 95% relative humidity. Upon heating the hydrate to 100° C., the sample converts to a new form (named Form H). Further heating of the sample from 100° C. to 140° C. shows that Form H then converts to Form A.

### Example 13

### Hydrate Form C XRPD Analysis

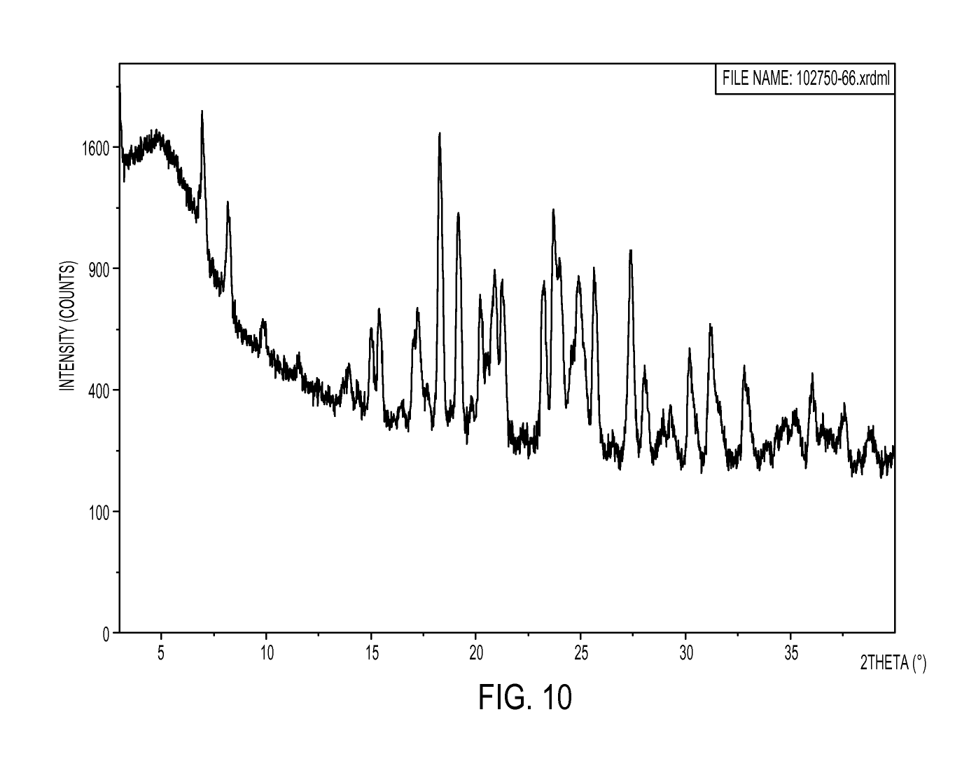

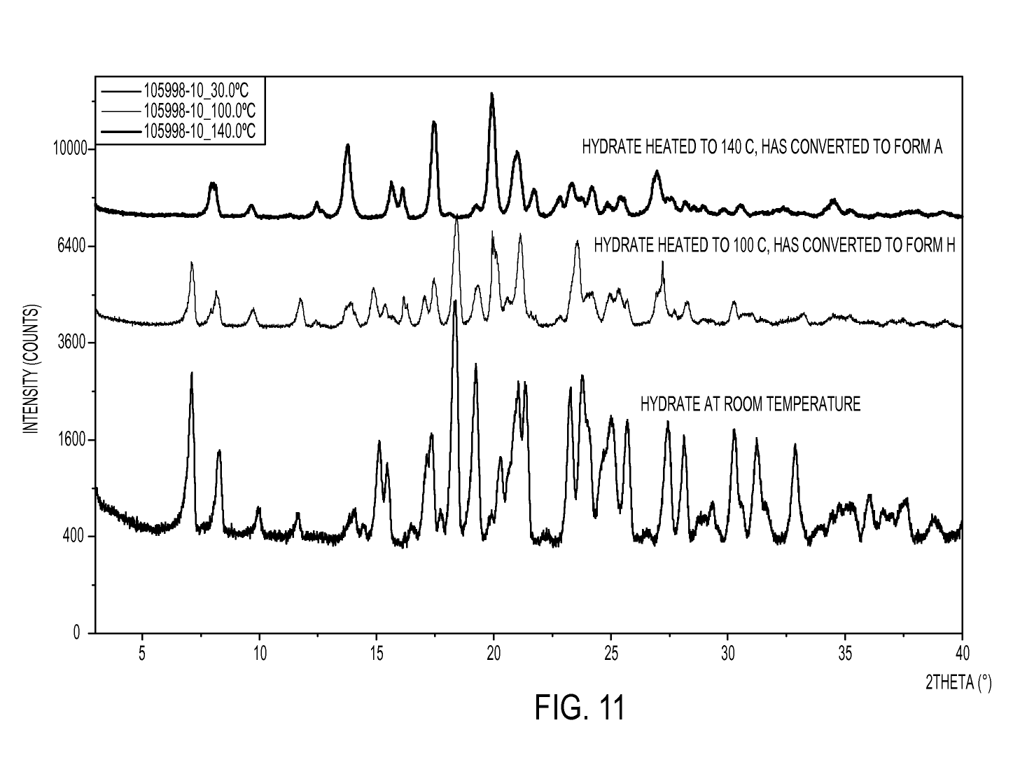

The XRPD pattern of Form C (mono-hydrate) is shown in FIG. 10. Form C has a distinct XRPD pattern having unique reflections at around 6.9, 8.2, 18.2, 19.2, 30.2 degrees 2θ. The XRPD pattern is characterized by sharp reflections, indicating crystallinity. To better understand the thermal events seen in the DSC, variable temperature XRPD was run on the hydrate. The data are shown in FIG. 11.

### Example 14

### Hydrate Form C Analysis

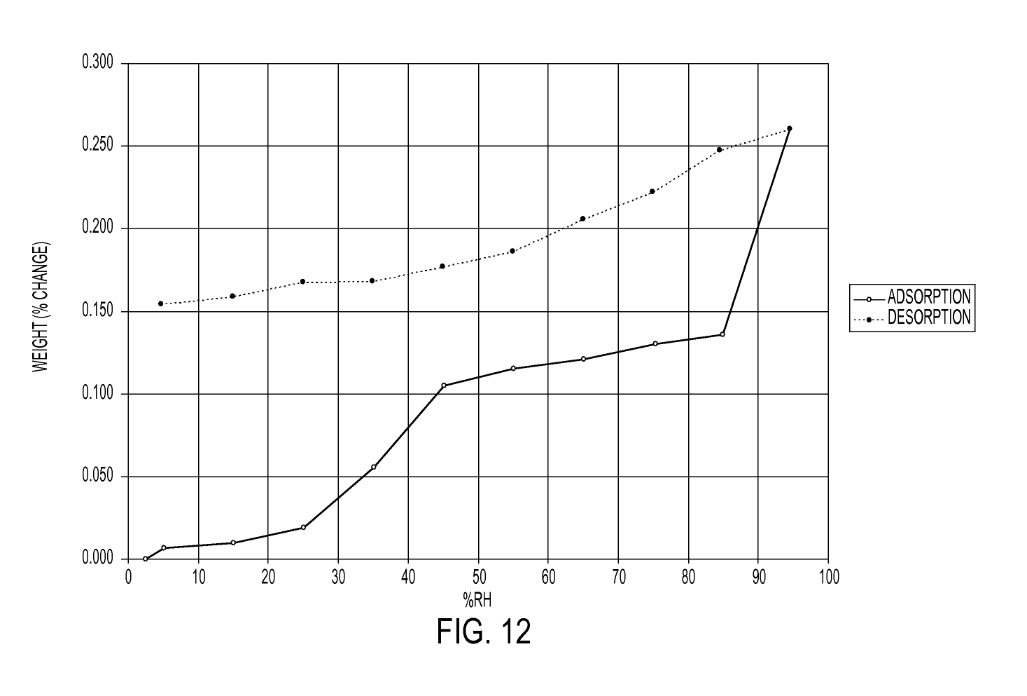

The moisture sorption curve for the hydrate is shown in FIG. 12 and indicates that the form is non-hygroscopic and only exhibits a 0.25% weight change as high as 95% relative humidity. The phase remained unchanged post experiment based on XRPD results.

### Example 15

### Free Base Form A

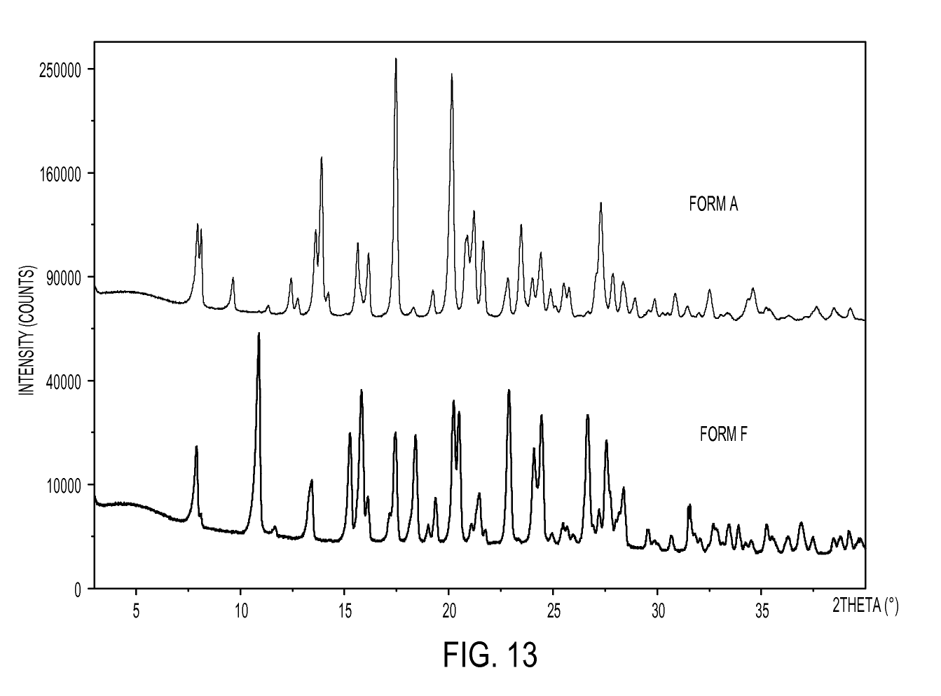

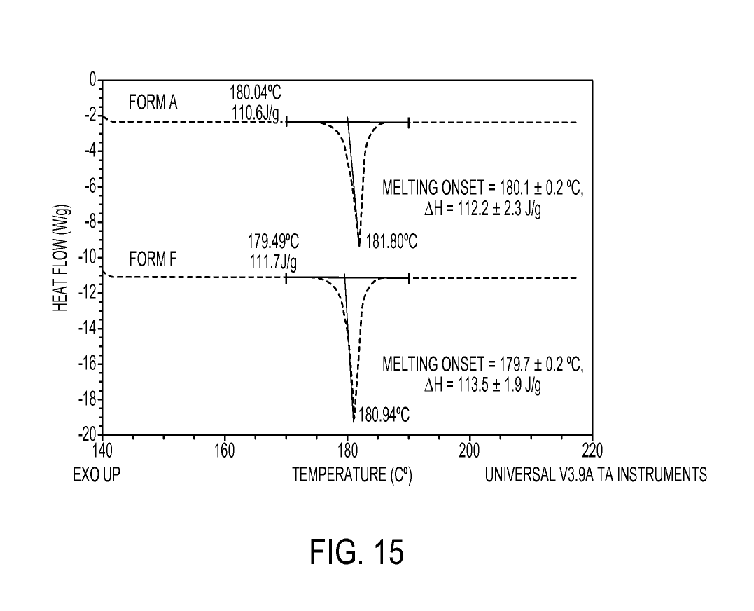

Form A was generated from a slow cooling process in 3:1 water:EtOH using seeds of pure Form A. Representative XRPD diffraction patterns for Form A are shown in FIG. 13. The XRPD patterns are characterized by sharp reflections indicating crystallinity and has an XRPD pattern distinct from Form F with Form A having unique reflections around 9.6, 12.4, 19.9, 20.1, 23.4 degrees 2θ. Form A was seen to have triangular blade-like morphology. Vapor sorption showed that both forms were non-hygroscopic and that each form remained unchanged post experiment based on XRPD results. Representative DSC curves for Form A are shown in FIG. 15. Form A was run separately five times on the DSC instrument at 10° C./min (FIG. 15) and the results were averaged. Form A was found to have melt onset temperature of 180.1±0.2° C. and a heat of melt of 112.2±2.3 J/g.

### Example 16

### Free Base Form F

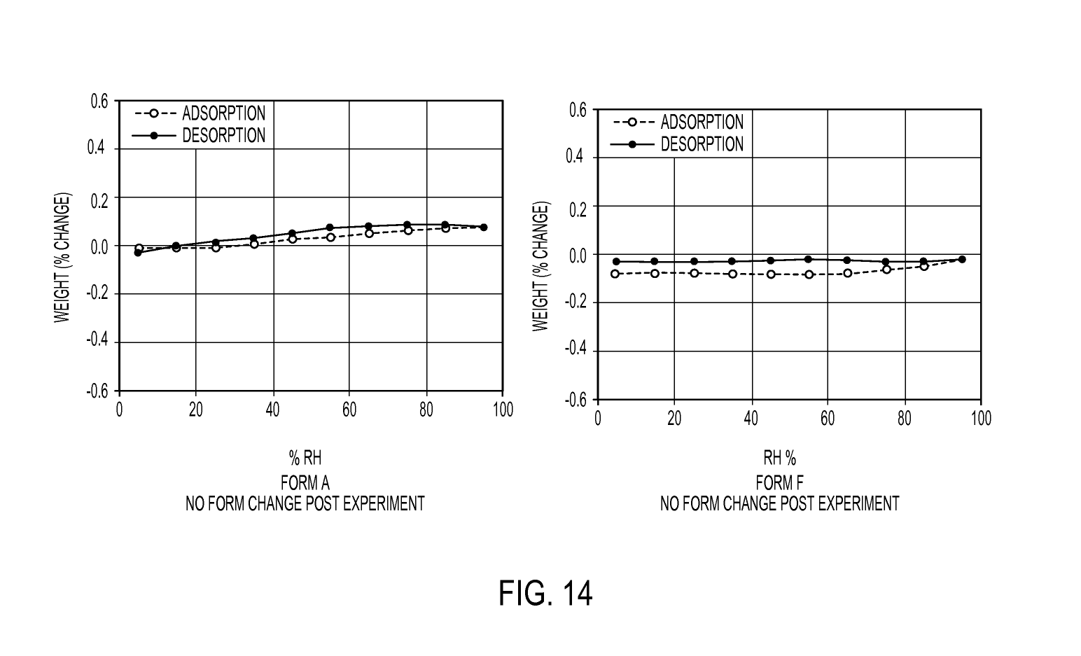

Form F was generated from a slow cooling process in MeOH using seeds of pure Form A. Representative XRPD diffraction patterns for Form F are shown in FIG. 13. The XRPD patterns are characterized by sharp reflections indicating crystallinity and has an XRPD pattern distinct from Form A with Form F having unique reflections around 10.8, 15.2, 15.8, 20.4, 26.7 degrees 2θ. Form F was found to have plate-like morphology. Vapor sorption showed that both forms were non-hygroscopic and that each form remained unchanged post experiment based on XRPD results. [FIG. 14]. Portions of a lot of Form F were run separately five times on the DSC instrument at 10° C./min (FIG. 15) and the results were averaged. Form F was found to have melt onset temperature of 179.7±0.2° C. and a heat of melt of 113.5±2.3 J/g.

Based on attempts to establish the thermodynamic relationship between Forms A and F, the two forms were found to be very similar and we were unable to determine the room temperature thermodynamic stable phase. Furthermore, attempts to scale up either phase by process chemistry through seeding resulted in mixtures or the wrong phase. It therefore was concluded that we could not control the forms.

### Example 17

### Salt Selection

A high through put and bench top salt and co-crystal screen were conducted on AMG151 and a summary of the experiments are listed in Tables 1-3 and in the results below. The solutions of H₂SO₄, H₃PO₄, L-Tartaric, Fumaric, Citric, and Maleic acid were prepared in methanol. A solution of HCl was prepared in IPA. Solutions of MeS03H, Benzenesulfonic and p-Toluenesulfonic acid were prepared in MeCN. The solution of succinic acid was prepared in water. The solid compound was dispensed into each well on a 96-well plate and the actual dispensed weights were recorded (see the table below for individual sample weight in milligram in each well). The calculated volume of acid standard solutions, except HCl, were dispensed into the 96-well source plate in a molar ratio of 1.10 (H₂SO₄, H₃SO₄, and methanesulfonic acid), and 1.05 (TSA, BSA, maleic, L-tartaric, fumaric, citric, and succinic) to the compound in the corresponding well, followed by evaporation of the solvent (MeOH, MeCN and water) under N2 stream using a 96-channel blower. Subsequently, the calculated volume of the HCl standard solution was manually dispensed into the source plate in a molar ratio of 1.50 to the compound in the corresponding wells. A stir disc was added to each well, and the source plate was sealed. For each experiment in the bench top screen, a portion of AMG151 was weighed into a vial and approximately 5-20 mL of a solvent was added. A counter ion was then added as a stoichiometric portion of an aqueous solution or straight solid and then the sample was sonicated for about 90 minutes and then the solvent was allowed to evaporate. Per library design, crystallization solvents were dispensed into the source plate (960 μL/well). After solvent addition, the source plate was sonicated for 30 minutes, then stirred and heated at 50° C. for 30 minutes. Upon continuous heating, the solvents in the source plate were aspirated, filtered at 50° C. into a filtration plate. The filtrate was subsequently aspirated and dispensed into three crystallization plates (evaporation, precipitation, cooling). After completion of 96-well filtration, the source plate was opened and kept stirring at 50° C. for 8 hours. The evaporation plate (200 μL/well filtrate) was left open at ambient for 24 hours. The sealed precipitation plate (150 μL/well filtrate injected into pre-filled 150 μL of n-butyl ether as anti-solvent) was cooled linearly from 25° C. to 5° C. in 8 hours and held at 5° C. for 8 hours. The sealed cooling plate (300 μL well filtrate) was started at 50° C., cubic cooled to 5° C. in 8 hours, and held at 5° C. for additional 8 hours. At the end of crystallization, the precipitation and cooling plates were centrifuged at 5° C. for 10 min at 1500 rpm, and the supernatant in each well of both plates was aspirated and discarded. Prior to dissembling each of 4 plates to collect the crystal samples on its 96-well glass substrates, wick paper was used to dip into each well to ensure the dryness. For the bench top sonic bath co-crystal screen, stoichiometric mixtures of AMG151 and co-crystal former were weighed into a centrifuge tube, a small amount of solvent was added and the wet powders were sonicated in a sonic bath for approximately 90 minutes.

TABLE 1
Salt and co-crystal screen of select counter ions for AMG151

| Counter Ion | Solvent | Observation |
|---|---|---|
| HCl | Acetone | Salt, Form A |
| | MeOH | Glass |
| | THF | Salt, Form B |
| | EtOH | Salt, Form C |
| | MeCN | Salt, Form D |
| H₃PO₄ | Acetone | Salt, Form A |
| | | Salt |
| | MeOH | Glass |
| | THF | Salt, Form B |
| H₂SO₄ | Acetone | Glass |
| | MeOH | Salt, Form A |
| | THF | Salt, Form B |
| Mandelic | Acetone | Form F of Free Form |
| | MeOH | Glass |
| | THF | Amorphous |
| Tartaric | Acetone | Amorphous |
| | MeOH | Glass |
| | THF | Amorphous |
| Citric | Acetone | Form F of Free Form |
| | MeOH | Glass |
| | THF | Glass |
| Maleic | Acetone | Glass |
| | MeOH | Glass |
| | THF | Amorphous |
| MSA | Acetone | Glass |
| | MeOH | Glass |
| | THF | Salt, Form A |
| | THF | Glass |
| BSA | Acetone | Glass |
| | MeOH | Glass |
| | THF | Glass |
| | THF | Glass |
| p-toluenesulfonic | Acetone | Glass |
| | MeOH | Glass |
| | THF | Amorphous |
| Fumarate | Acetone | Salt, Form A |
| | MeOH | Salt, Form A |
| | THF | Salt, Form A |
| Succinic | Acetone | Salt, Form A |
| | MeOH | Amorphous |
| | THF | Amorphous |
| Acetic | THF | Amorphous |
| Lactic | THF | Amorphous |
| Malic | THF | Amorphous |
| Malonic | THF | Amorphous |
| SLS | THF | Amorphous |
| Oxalic | THF | Amorphous |
| Stearic | THF | Amorphous |

TABLE 2
Summary of salt screening slurry experiments

| Counter Ion | Solvent | Observation |
|---|---|---|
| Mandelic | Water Slurry | Hydrate of Free Form |
| Tartaric | Water Slurry | Hydrate of Free Form |
| Maleic | Water Slurry | Hydrate of Free Form |
| H₂PO₄ | Water Slurry | From F of Free Form |
| H₂PO₄ | Water Slurry | Form A of Free Form |
| HCl | Water Slurry | Form A of Free Form |
| Fumarate | Water Slurry | Salt, no form change |
| H₂PO₄ | EtOH | Form A of Free Form |
| H₂PO₄ | MeCN | MeCN solvate of Free Form |
| HCl | EtOH | Solvate of Salt |
| HCl | MeCN | Solvate of Salt |

TABLE 3
Summary of sonic bath co-crystal screening experiments

| Counter Ion | Solvent | Co Crystal Result |
|---|---|---|
| Tartaric | THF | No |
| | MeOH | No |
| Citric | THF | No |
| | MeOH | No |
| Maleic | THF | No |
| | MeOH | No |
| Mandelic | THF | No |
| | MeOH | No |
| Stearic | THF | No |
| | MeOH | No |
| Succinic | THF | No |
| | MeOH | No |

A. HCl Salt

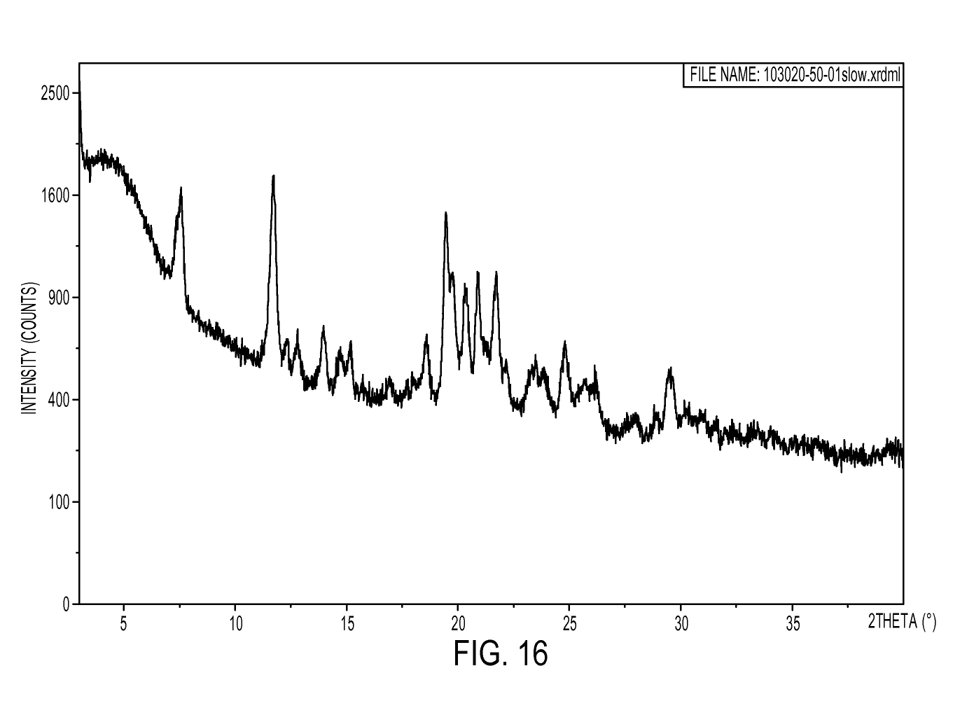

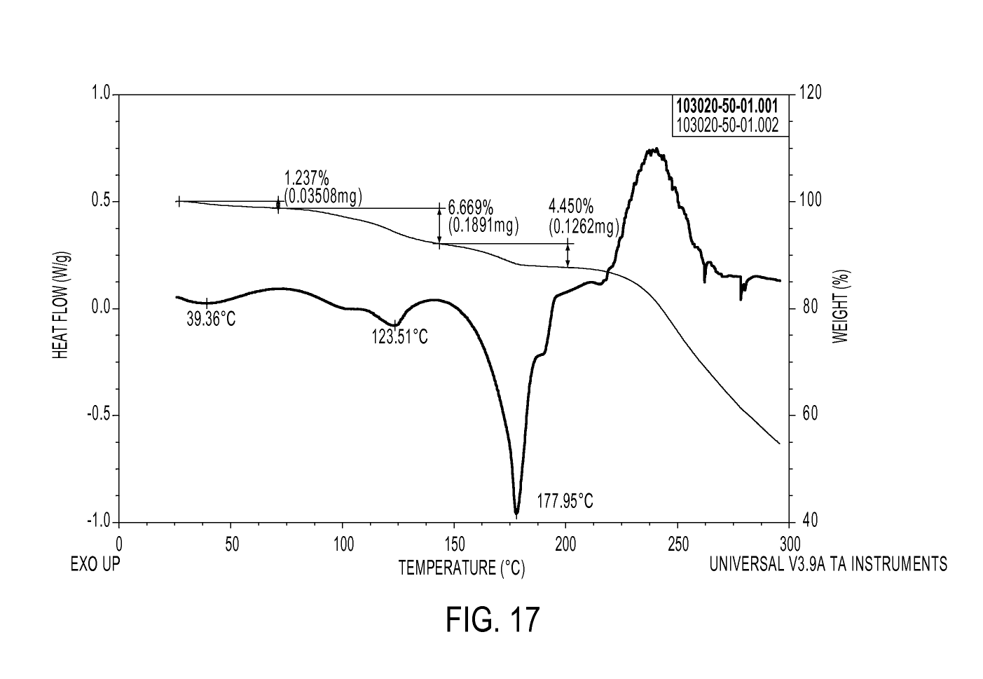

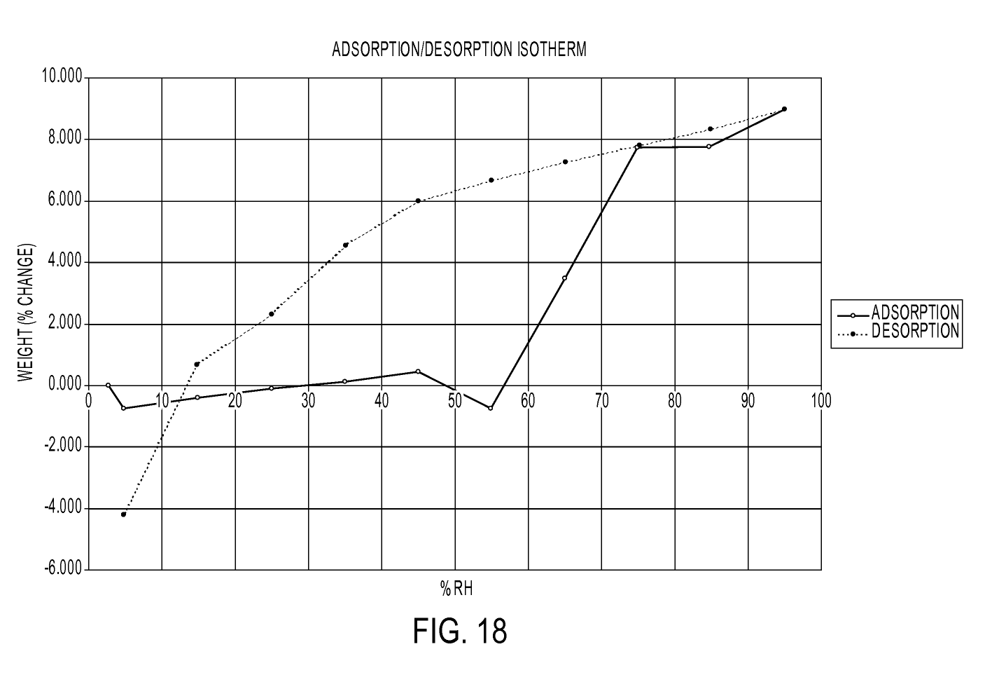

The HCl salt Form A material was generated from acetone, and a representative XRPD pattern is shown in FIG. 16. The sample appears to be crystalline with an irregular morphology. This phase was further characterized via DSC and TGA and the data are shown in FIG. 17. The DSC curve indicates broad endotherms around 39 and 123° C. (attributed to desolvation), and one sharp endotherm around 178° C. (attributed to a melt). The TG curve indicates a 12.2% weight loss up to 200° C. The vapor sorption curve is shown in FIG. 18 and indicates that the material is hygroscopic above 55% relative humidity and exhibits a 9% weight change as high as 95% relative humidity. The material loses this weight upon drying down to 5% relative humidity and the sample was shown to have converted to an amorphous phase post experiment based on XRPD results. This phase had a complicated thermal behavior.

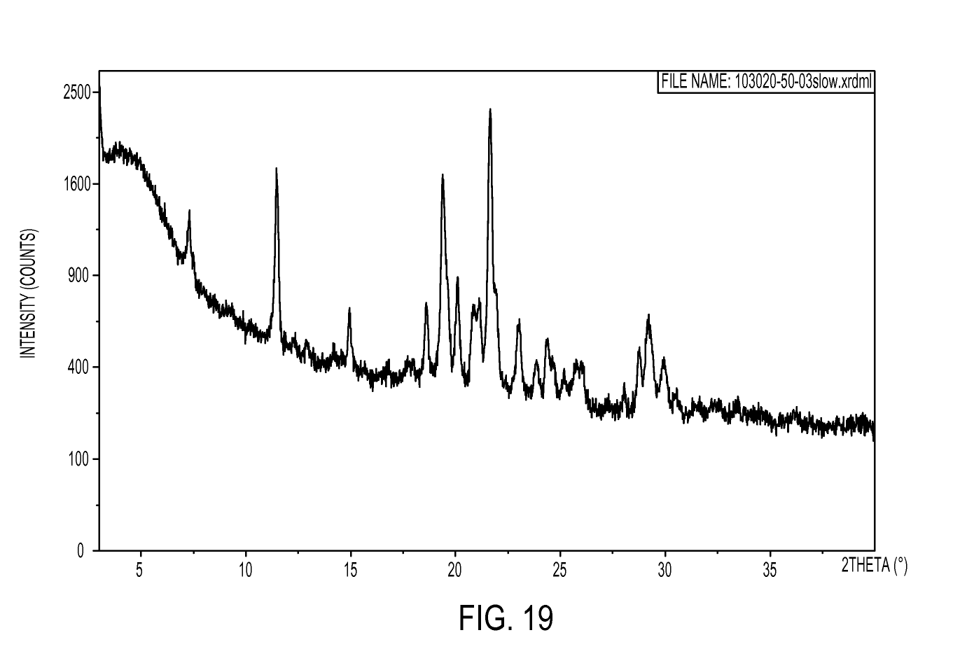

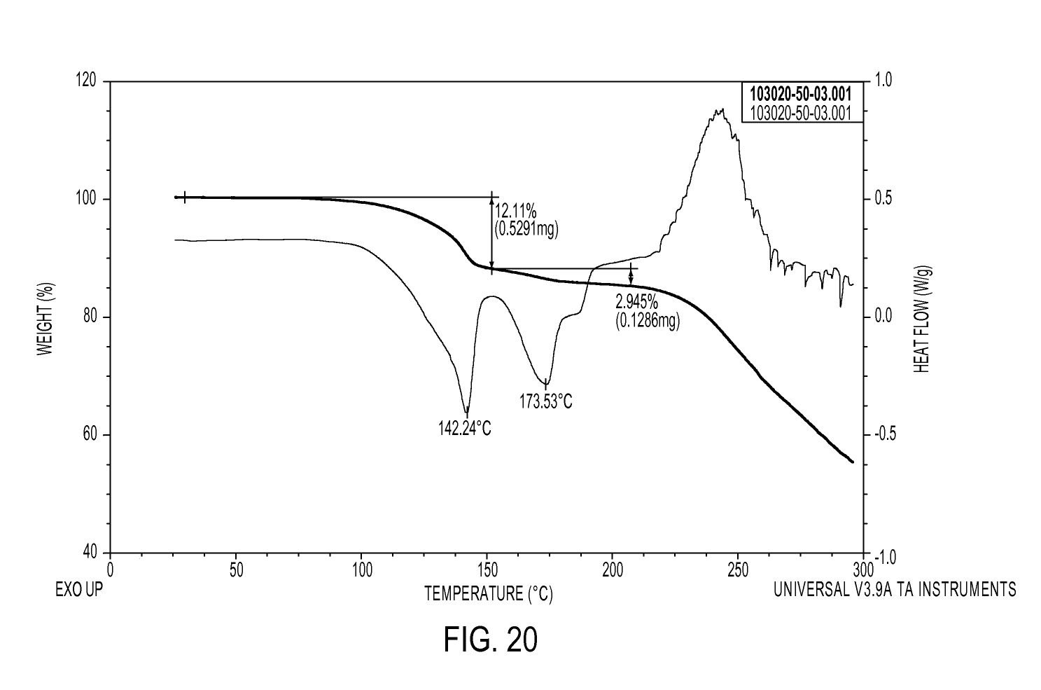

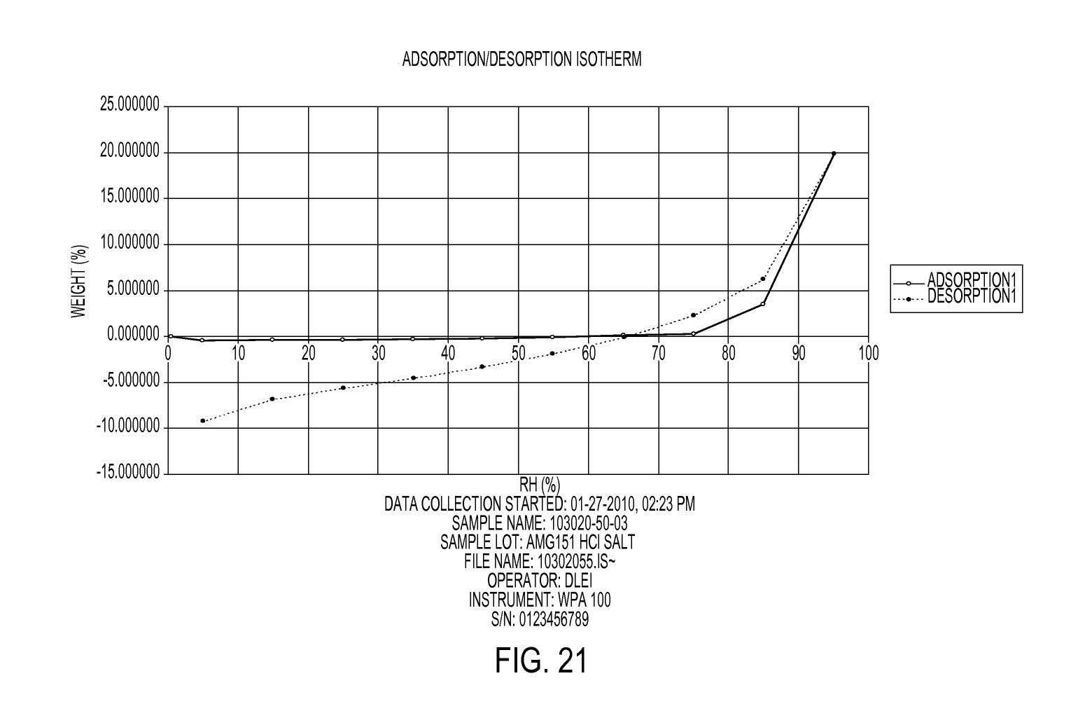

The HCl salt Form B material was generated from THF, and a representative XRPD pattern is shown in FIG. 19. The sample appears to be crystalline with an irregular morphology. This phase was further characterized via DSC and TGA and the data are shown in FIG. 20. The DSC curve indicates broad endotherms around 142 (attributed to desolvation) and 174° C. (attributed to a melt). The TG curve indicates a 15% weight loss up to 215° C. The vapor sorption curve is shown in FIG. 21 and indicates that the material is hygroscopic above 75% relative humidity and exhibits a 20% weight change as high as 95% relative humidity. The material loses this weight upon drying down to 5% relative humidity and the sample was shown to have converted to an amorphous phase post experiment based on XRPD results. This phase had a complicated thermal behavior.

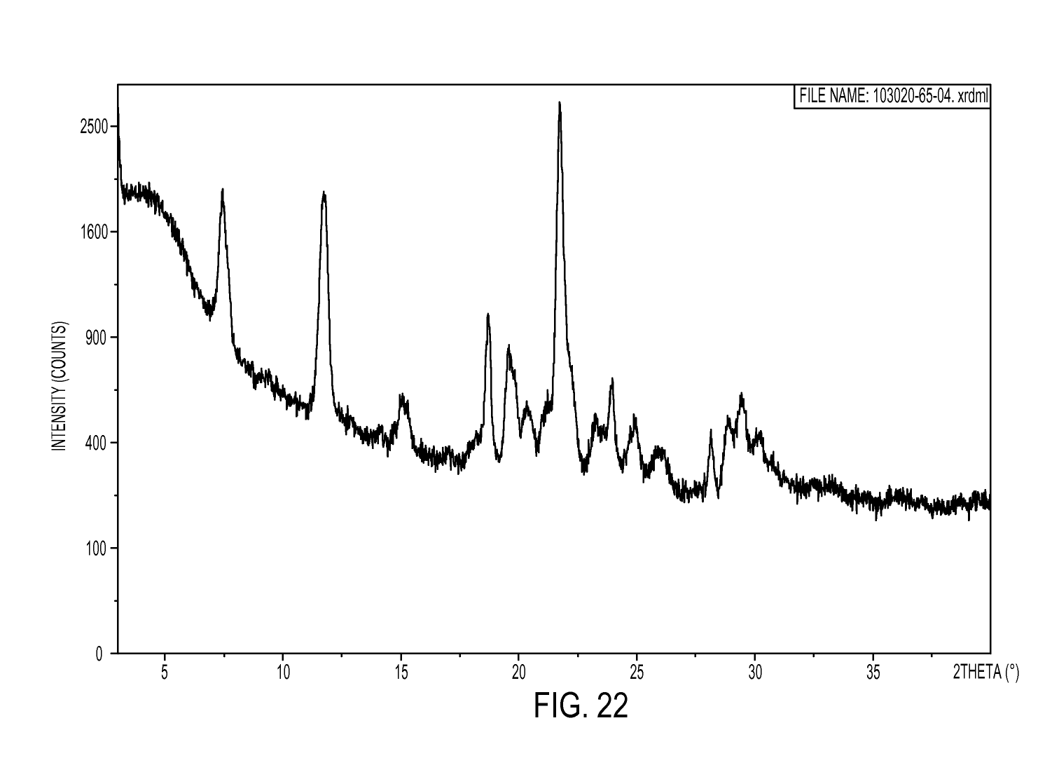

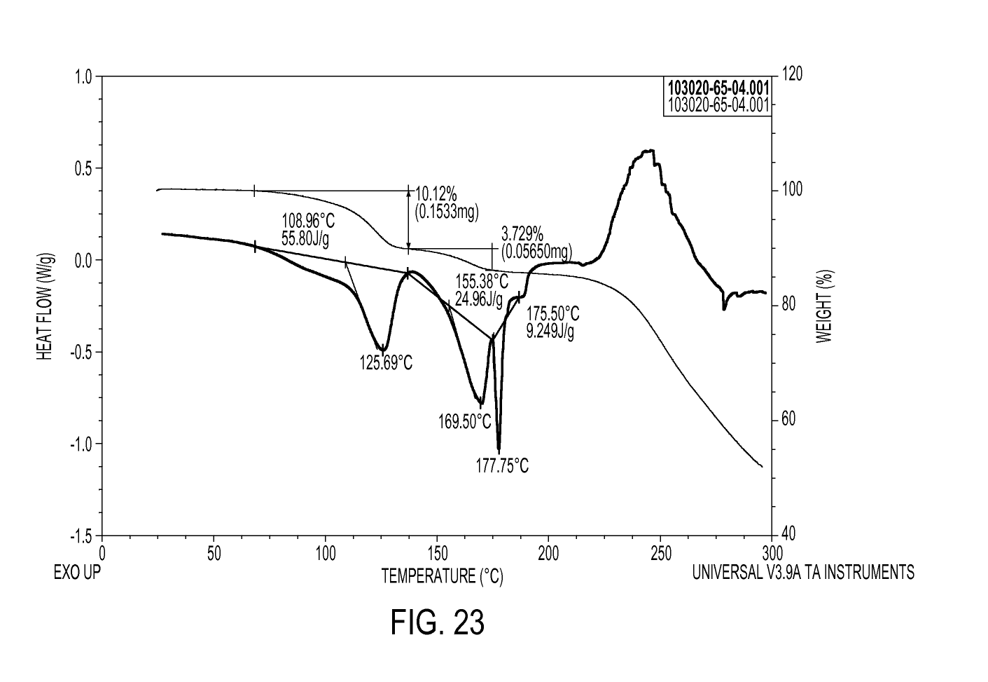

The HCl salt Form C material was generated from EtOH, and a representative XRPD pattern indicates that the material is crystalline and is shown in FIG. 22. This phase was further characterized via DSC and TGA and the data are shown in FIG. 23. The DSC curve indicates broad endotherms around 126 and 170° C. (attributed to desolvation), and one sharp endotherm around 177° C. (attributed to a melt). The TG curve indicates a 13.8% weight loss up to 175° C. This phase had a complicated thermal behavior.

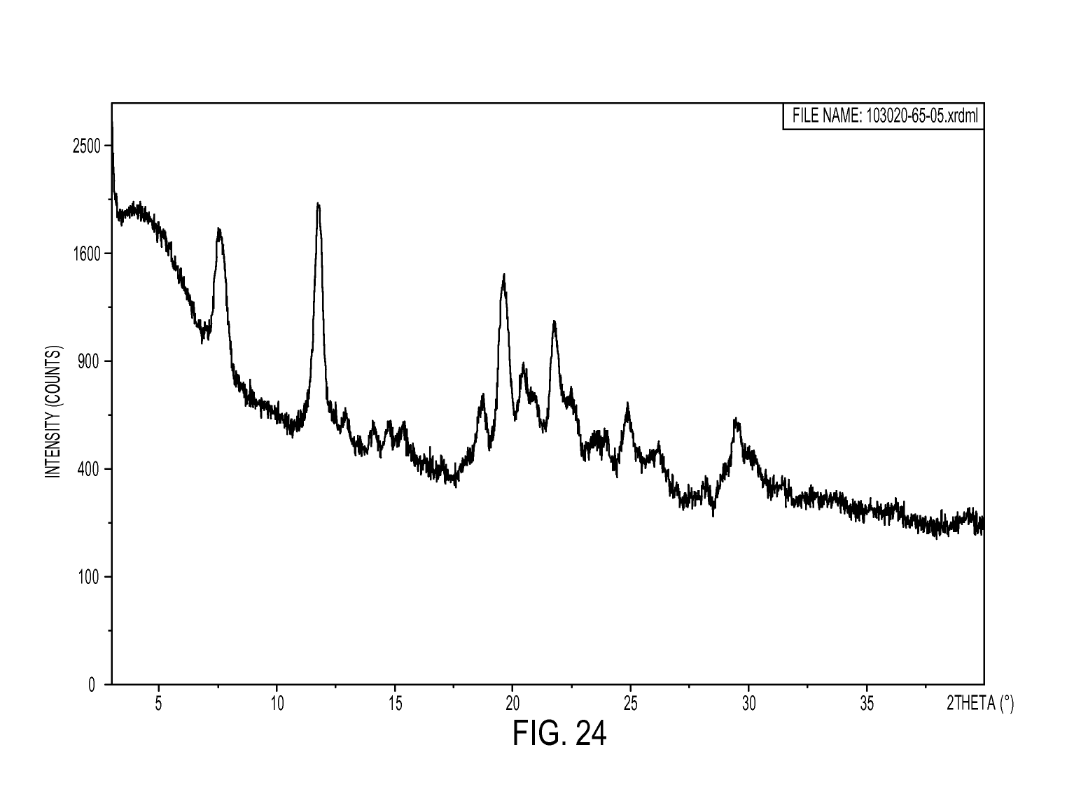

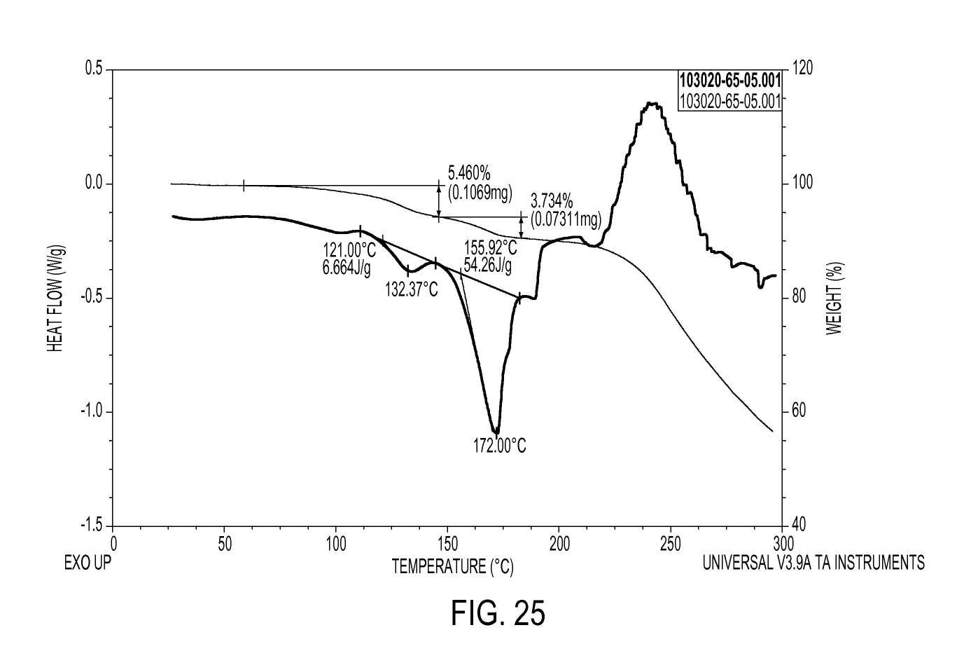

The HCl salt Form D material was generated from MeCN, and a representative XRPD pattern indicates that the material is crystalline and is shown in FIG. 24. This phase was further characterized via DSC and TGA and the data are shown in FIG. 25. The DSC curve indicates broad endotherms around 132 (attributed to desolvation) and 172° C. (attributed to desolvation with a melt). The TG curve indicates a 9.1% weight loss up to 175° C. Due to the complicated thermal behavior of this phase it was not pursued.

B. Phosphate Salt

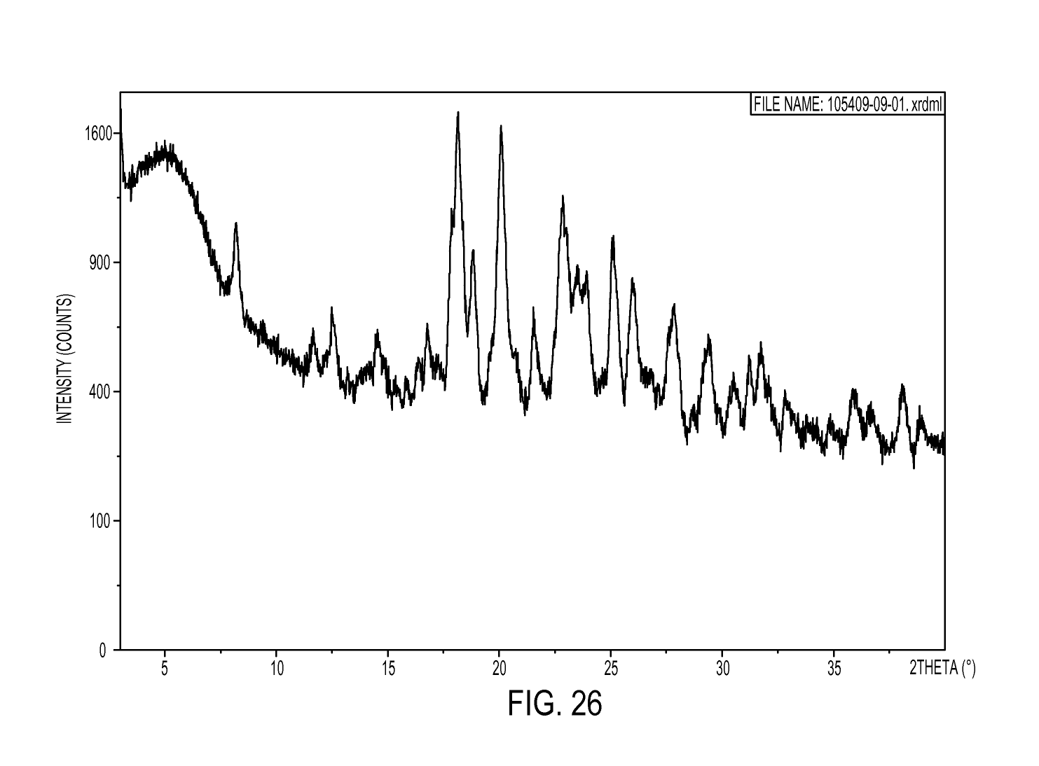

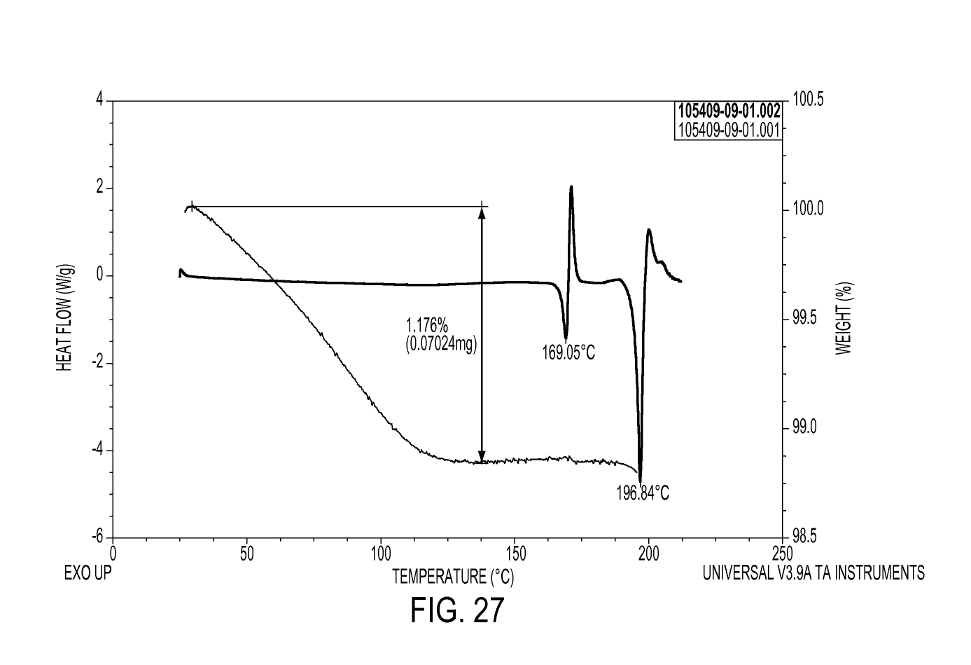

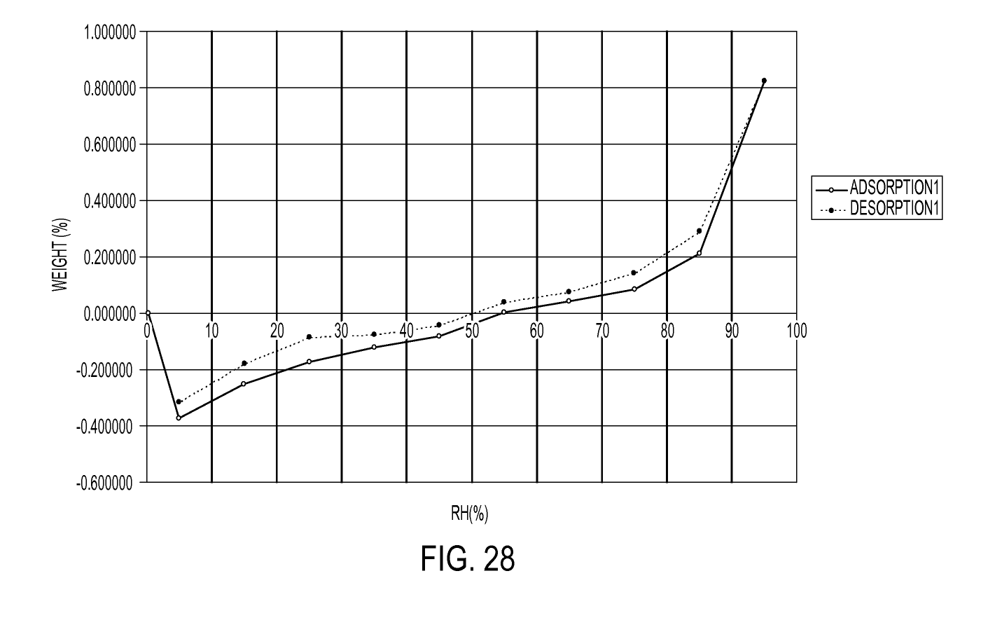

The phosphate salt Form A material was generated from acetone and from exposing solvated forms of the phosphate salt to high relative humidity. A representative XRPD pattern is shown in FIG. 26. The sample appears to be crystalline with an irregular morphology. This phase was further characterized via DSC and TGA and the data are shown in FIG. 27. The DSC curve indicates sharp endotherms around 169 (attributed to conversion to From D) and 196° C. (attributed to a melt). The TG curve indicates a 1.2% weight loss up to 135° C. Karl Fischer analysis yielded 1.8% water, indicating that this is a hemi-hydrate of the phosphate salt (1.6% for theoretical hemi-hydrate). The vapor sorption curve is shown in FIG. 28 and indicates that the sample partially desolvated when dried to 5% relative humidity, but the material fully re-hydrates by 55% relative humidity. Above 55% relative humidity, the sample is non-hygroscopic and only exhibits a 0.8% weight change as high as 95% relative humidity. The phase remained unchanged post experiment based on XRPD results. This phase was found to be crystalline, non-hygroscopic and a hydrate that readily resorbs water upon desolvation.

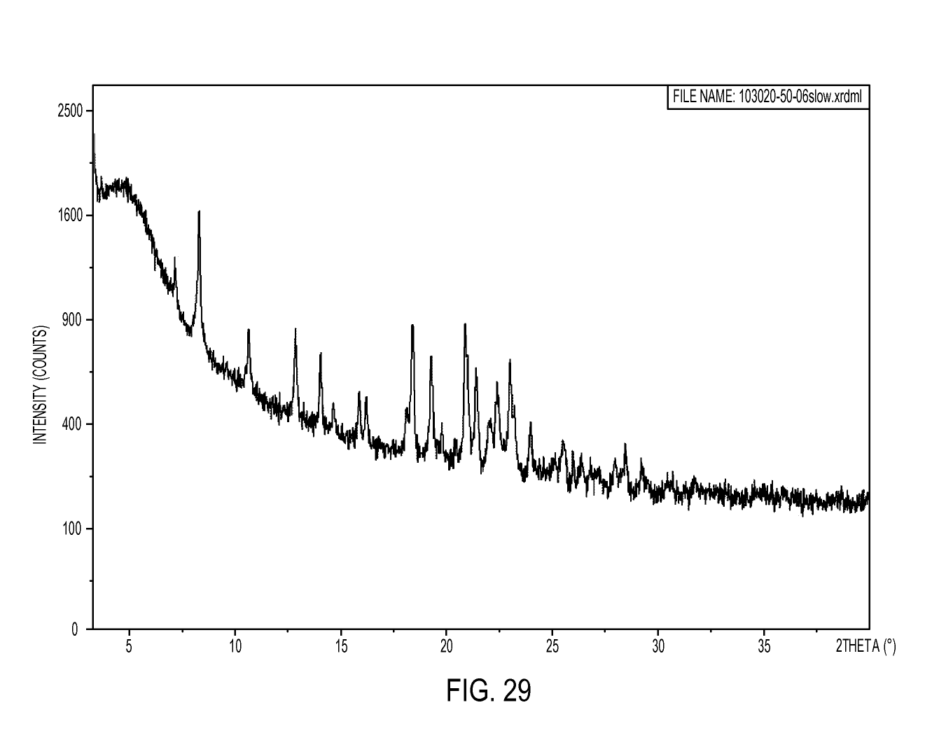

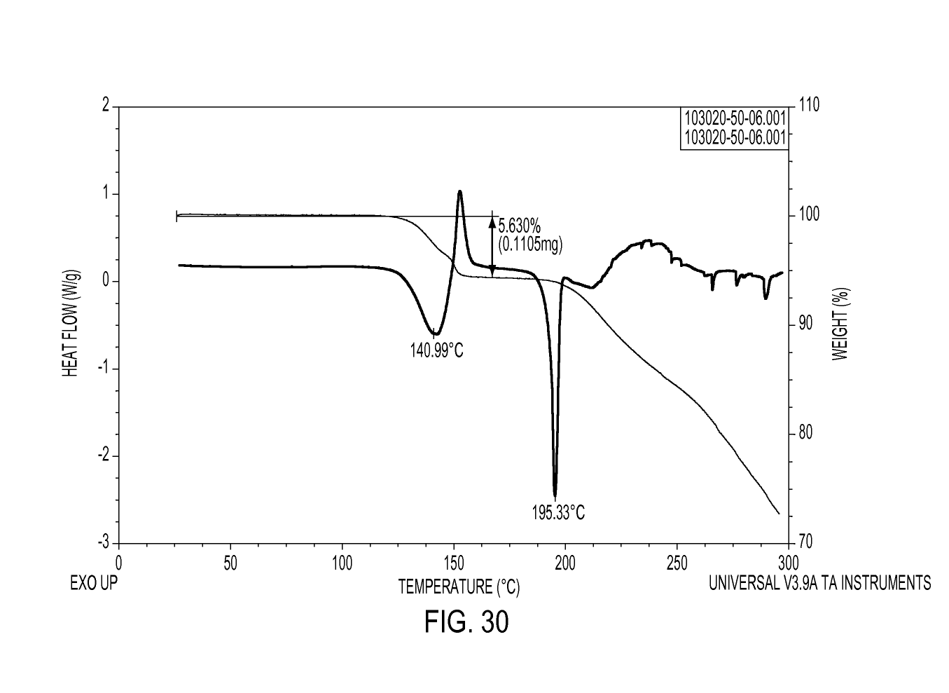

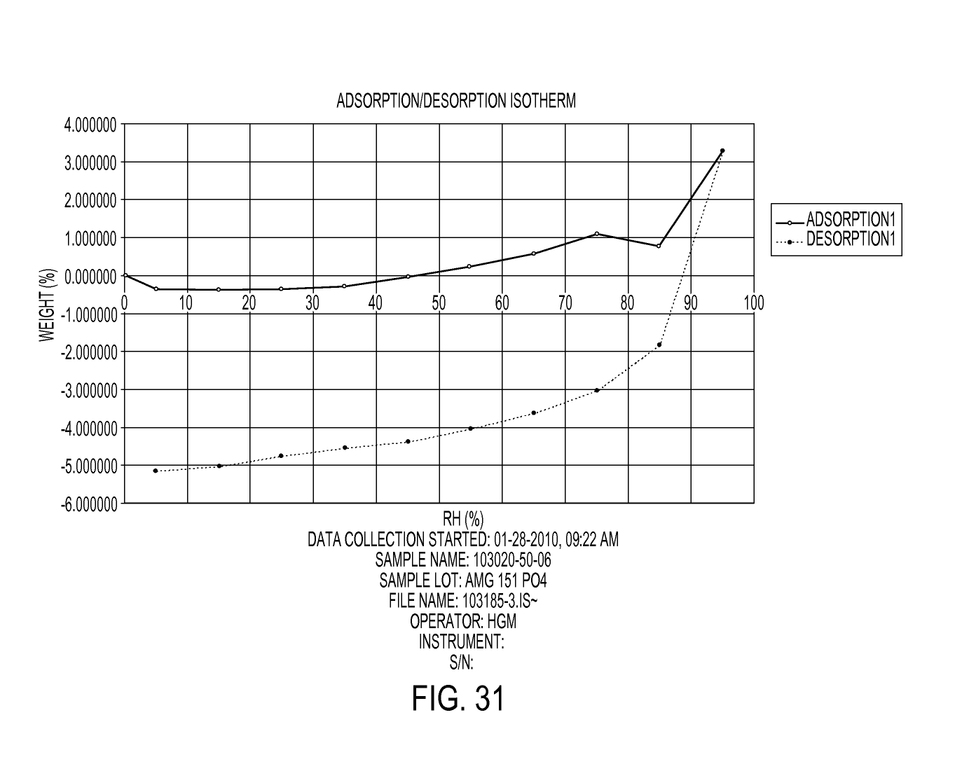

The phosphate salt Form B material was generated from THF, and a representative XRPD pattern is shown in FIG. 29. The sample appears to be crystalline with an irregular morphology. This phase was further characterized via DSC and TGA and the data are shown in FIG. 30. The DSC curve indicates a broad endotherm around 140° C. (attributed to desolvation), and one sharp endotherm around 195° C. (attributed to a melt). The TG curve indicates a 5.6% weight loss up to 175° C. The vapor sorption curve is shown in FIG. 31 and indicates that the material is slightly hygroscopic and exhibits a 3.5% weight change as high as 95% relative humidity; however, upon drying down to 5% relative humidity, the sample loses 8.5%. The phase was found to have converted to Form A post experiment based on XRPD results. This phase was unstable as a function of ambient relative humidity.

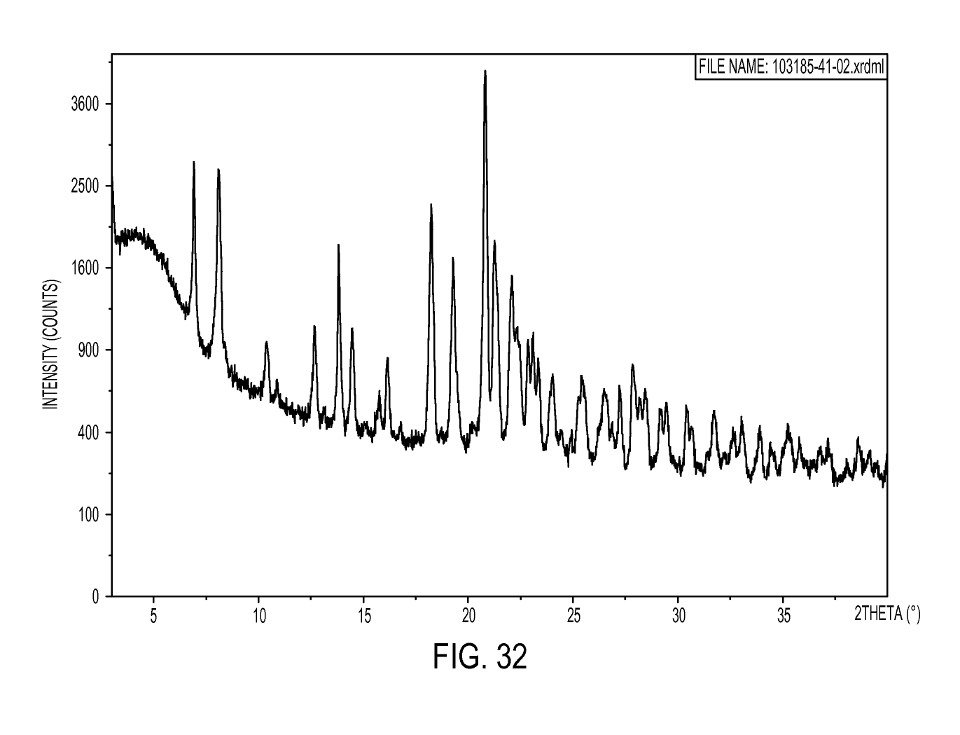

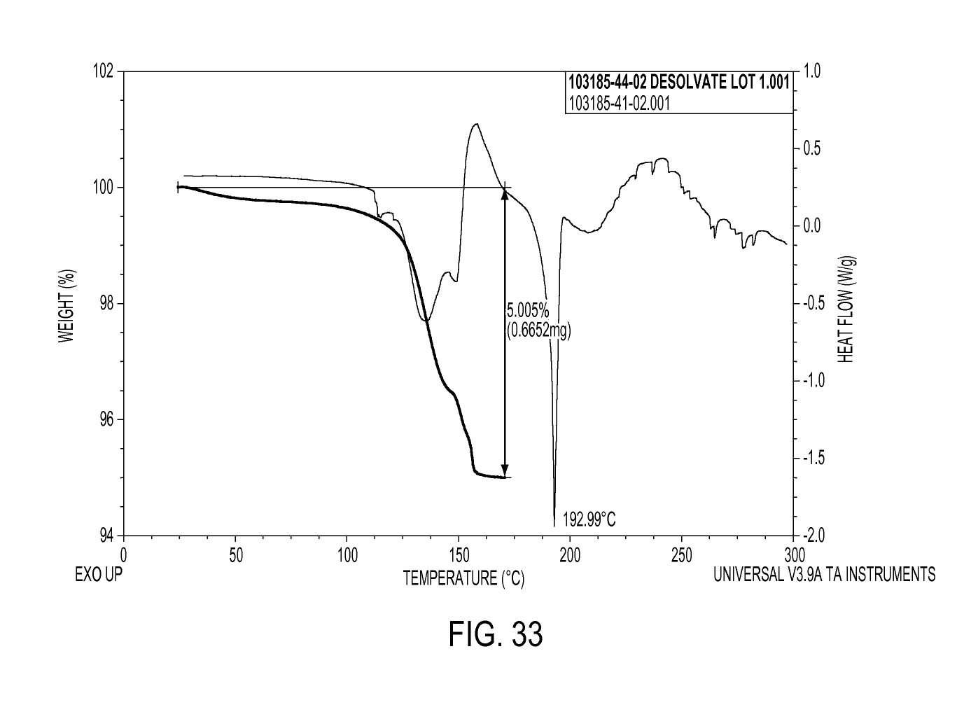

The phosphate salt Form C material was generated from acetone, and a representative XRPD pattern indicates that the material is crystalline and is shown in FIG. 32. This phase was further characterized via DSC and TGA and the data are shown in FIG. 33. The DSC curve indicates a broad endotherm around 150° C. (attributed to desolvation), and one sharp endotherm around 193° C. (attributed to a melt). The TG curve indicates a 5.0% weight loss up to 175° C. When a portion of this phase was exposed to a 95% relative humidity chamber, it was found to have converted to Form A based on XRPD results. When a portion of this phase was heated to 175° C. and then x-rayed, it was found to have converted to Form D. This phase was unstable as a function of ambient relative humidity.

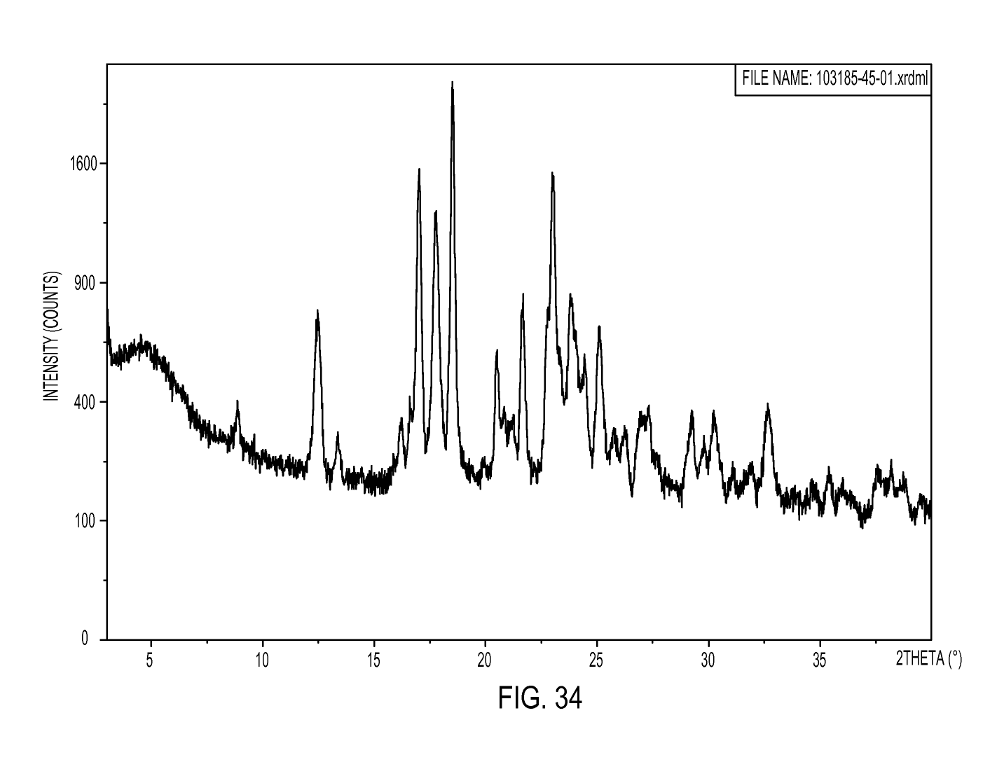

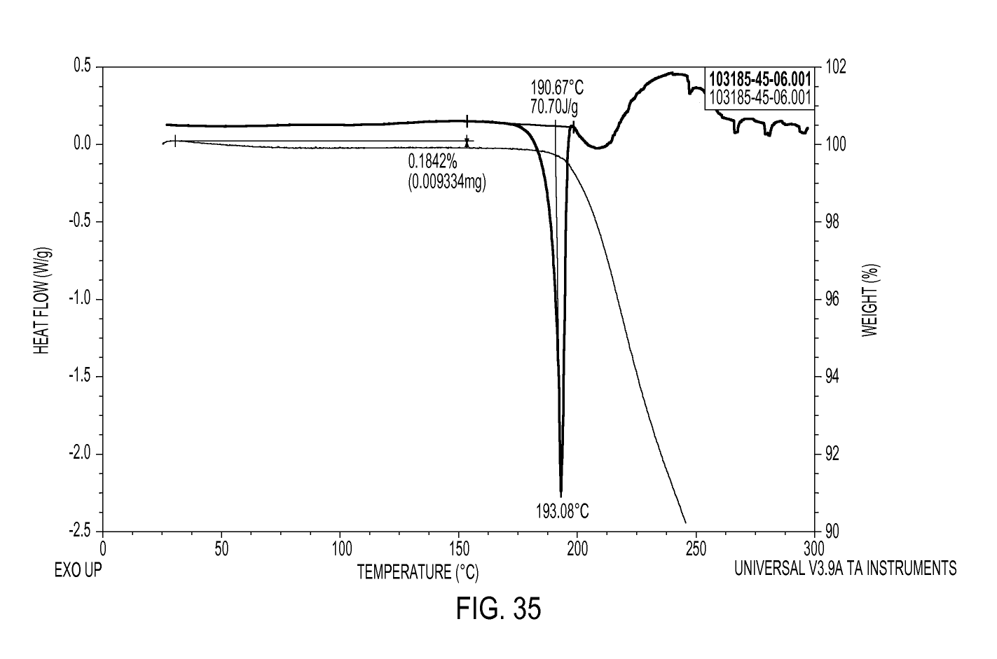

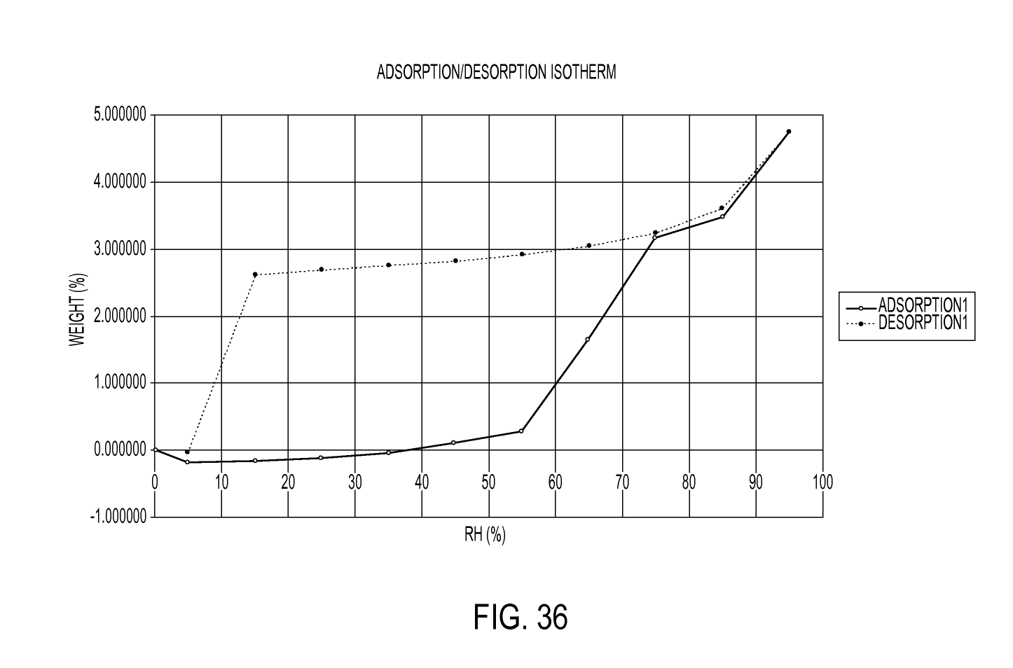

The phosphate salt Form D material was generated from desolvating Form C, and a representative XRPD pattern is shown in FIG. 34. The sample appears to be crystalline with an irregular morphology. This phase was further characterized via DSC and TGA and the data are shown in FIG. 35. The DSC curve indicates a sharp endotherm around 193° C. (attributed to a melt). The TG curve indicates a 0.2% weight loss up to 150° C. indicating that the form is unsolvated. The vapor sorption curve is shown in FIG. 36 and indicates that the material is hygroscopic above 55% relative humidity and appears to convert to a mono-hydrate around 75% relative humidity as it exhibits a 3.0% weight change at this condition. The material appears to retain this sorbed water upon drying down to 15% relative humidity; however, upon reaching 5% relative humidity the material losses all of its sorbed water and returns to dry weight. The phase remained unchanged post experiment based on XRPD results. When a portion of this phase was placed in a 95% relative humidity jar and then removed and immediately x-rayed, it was found to convert to Form E. This phase was unstable as a function of ambient relative humidity.

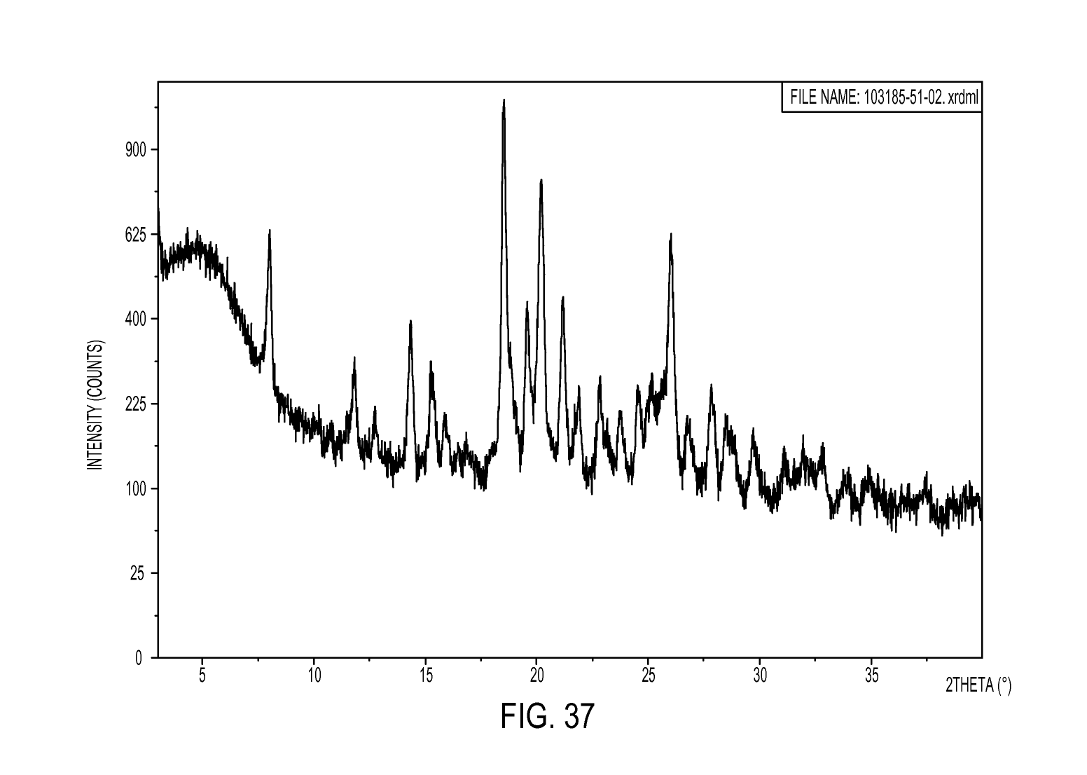

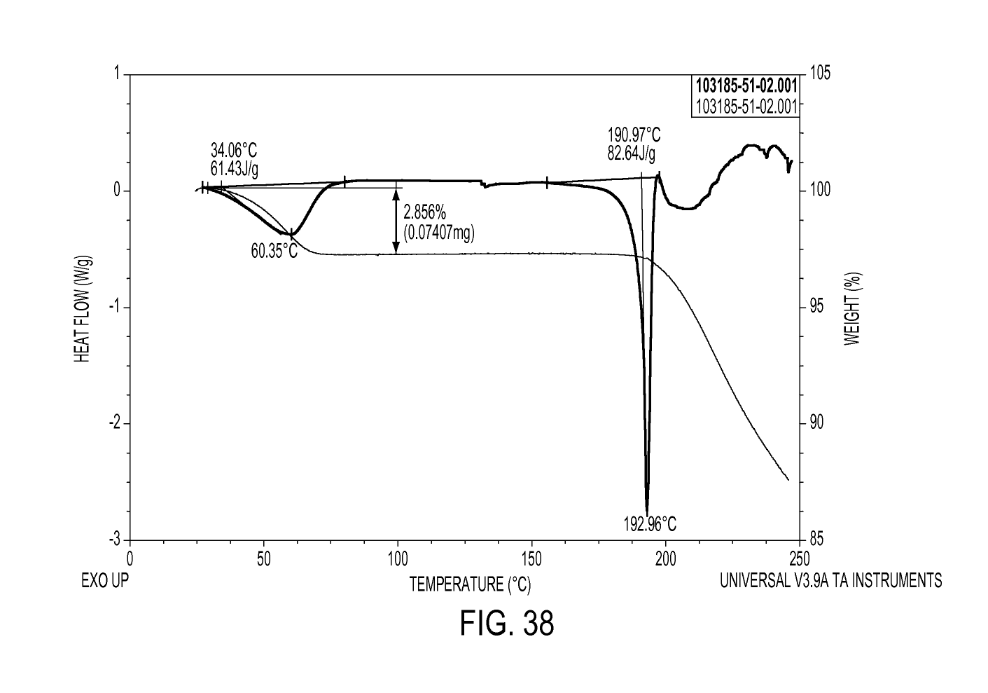

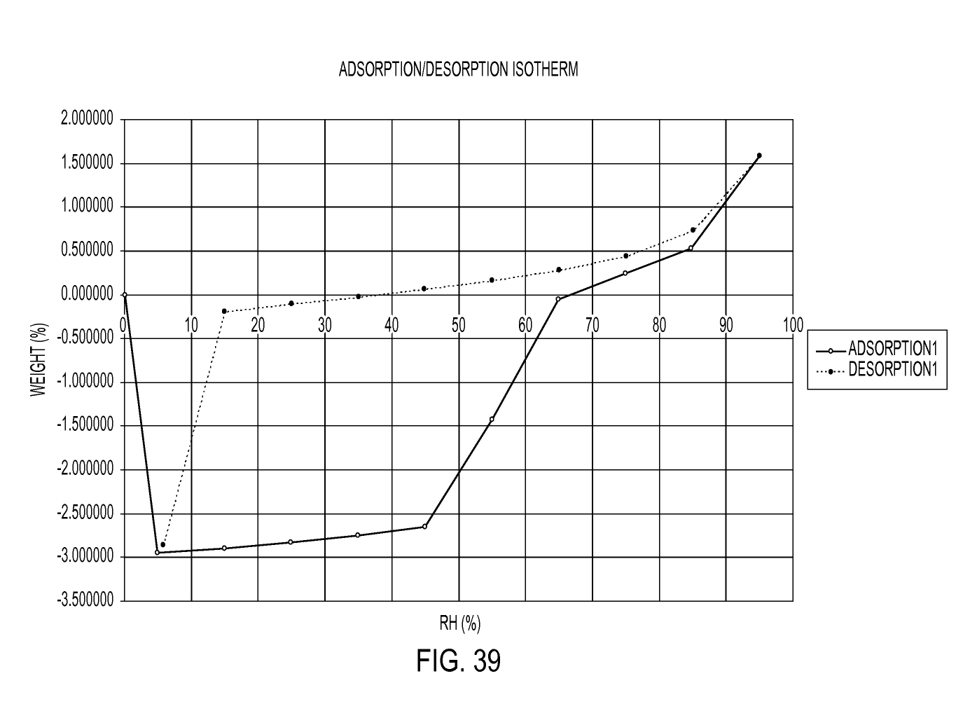

The phosphate salt Form E material was generated from exposing Form D to high relative humidity, and a representative XRPD pattern is shown in FIG. 37. The sample appears to be crystalline with an irregular morphology. This phase was further characterized via DSC and TGA and the data are shown in FIG. 38. The DSC curve indicates a broad endotherm around 60° C. (attributed to desolvation), and one sharp endotherm around 193° C. (attributed to a melt). The TG curve indicates a 2.9% weight loss up to 100° C. Karl Fischer analysis yielded 3.0% water, indicating that this is a mono-hydrate of the phosphate salt (3.2% for theoretical hemi-hydrate). The vapor sorption curve is shown in FIG. 39 and indicates that the material desolvated when dried to 5% relative humidity, but the material fully re-hydrates by 65% relative humidity. Above 65% relative humidity, the sample is slightly hygroscopic. This phase was unstable as a function of ambient relative humidity.

C. Sulfate Salt

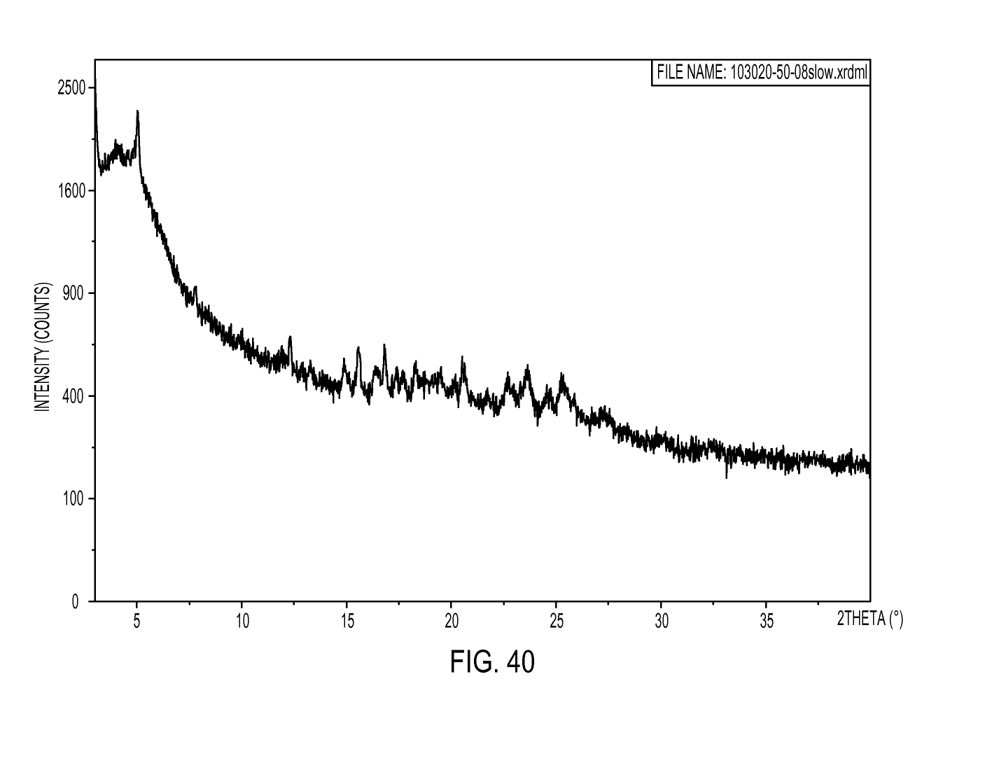

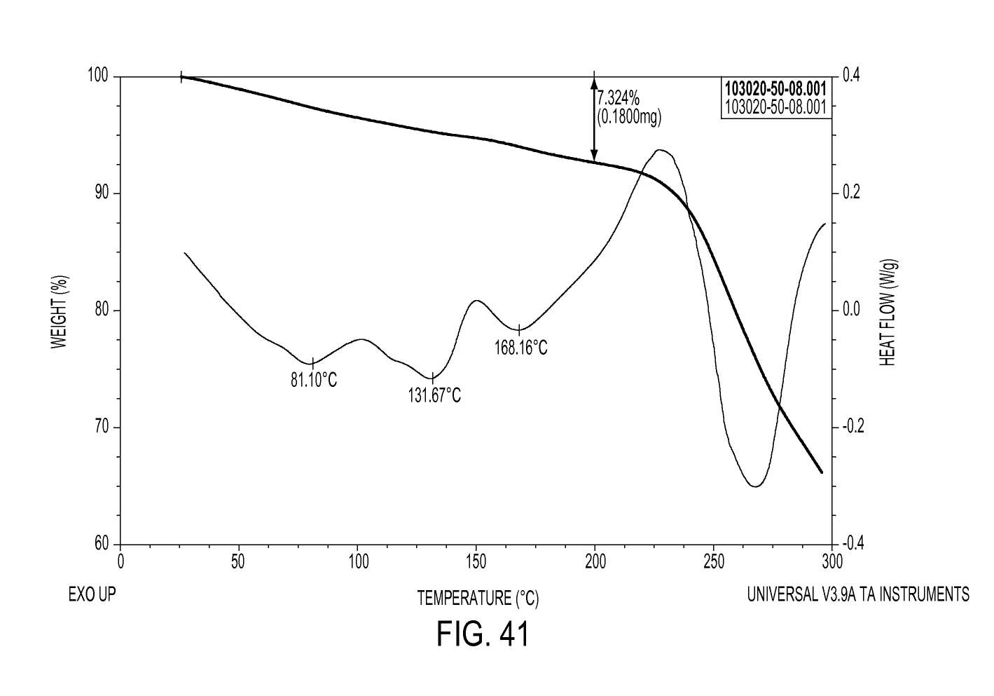

The sulfate salt Form A material was generated from MeOH, and a representative XRPD pattern is shown in FIG. 40. The sample appears to be crystalline with an irregular morphology. This phase was further characterized via DSC and TGA and the data are shown in FIG. 41. The DSC curve indicates broad endotherms around 81, 132 and 168° C. (attributed to desolvation and possibly a melt). The TG curve indicates a 7.3% weight loss up to 200° C. This phase has a complicated thermal behavior.

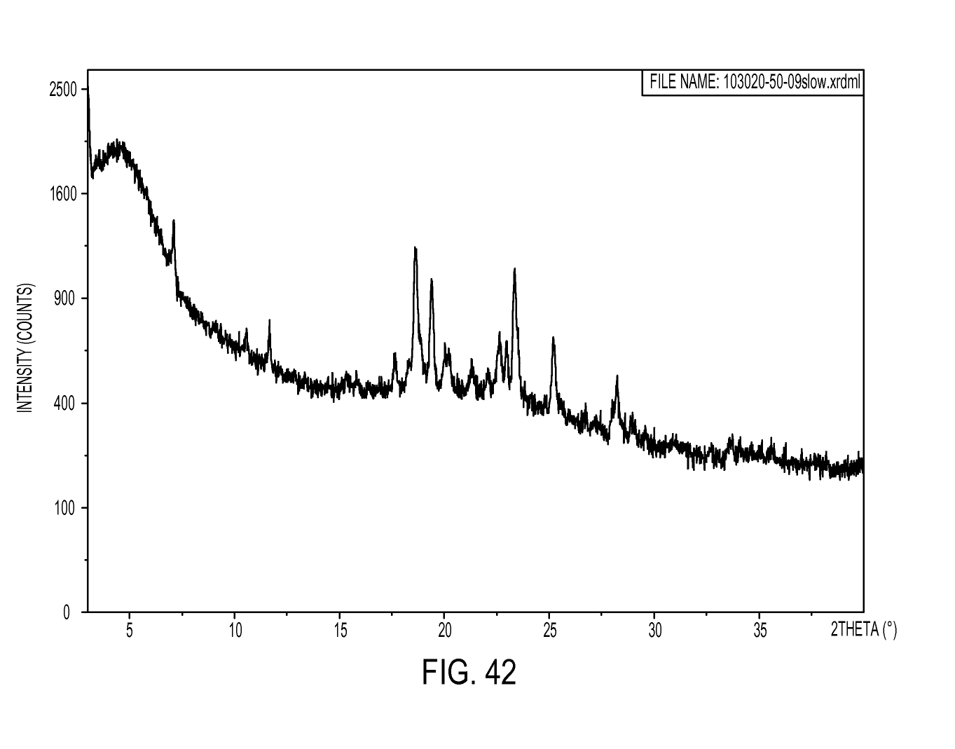

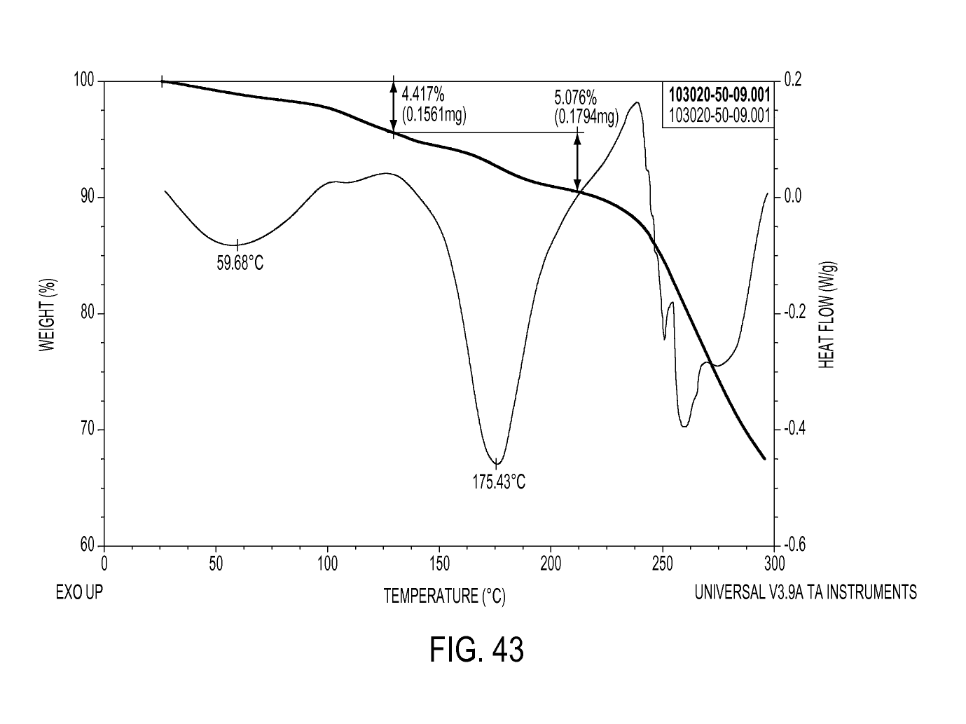

The sulfate salt Form B material was generated from THF, and a representative XRPD pattern is shown in FIG. 42, respectively. The sample appears to be crystalline with an irregular morphology. This phase was further characterized via DSC and TGA and the data are shown in FIG. 43. The DSC curve indicates broad endotherms around 59° C. (attributed to desolvation) and 175° C. (attributed to a melt). The TG curve indicates a 9.4% weight loss up to 215° C. This phase has a complicated thermal behavior.

D. Methanesulfonic acid Salt

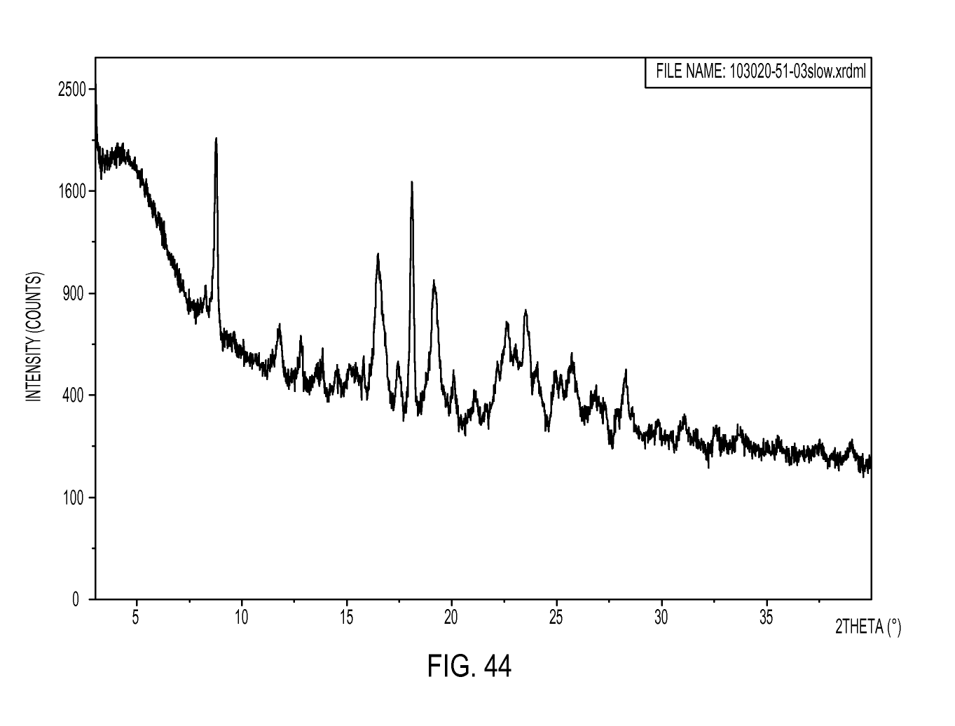

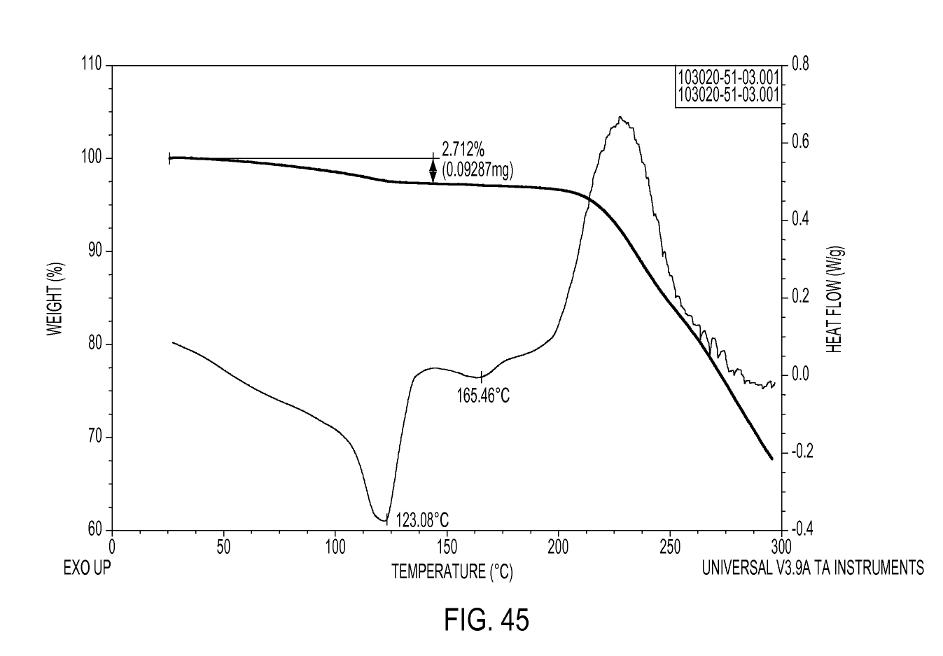

The methanesulfonic acid (MSA) salt Form A material was generated from THF, and a representative XRPD pattern is shown in FIG. 44. The sample appears to be crystalline with an irregular morphology. This phase was further characterized via DSC and TGA and the data are shown in FIG. 45. The DSC curve indicates broad endotherms around 123 and 165° C. (attributed to desolvation and possibly a melt). The TG curve indicates a 2.7% weight loss up to 150° C. This phase has a complicated thermal behavior.

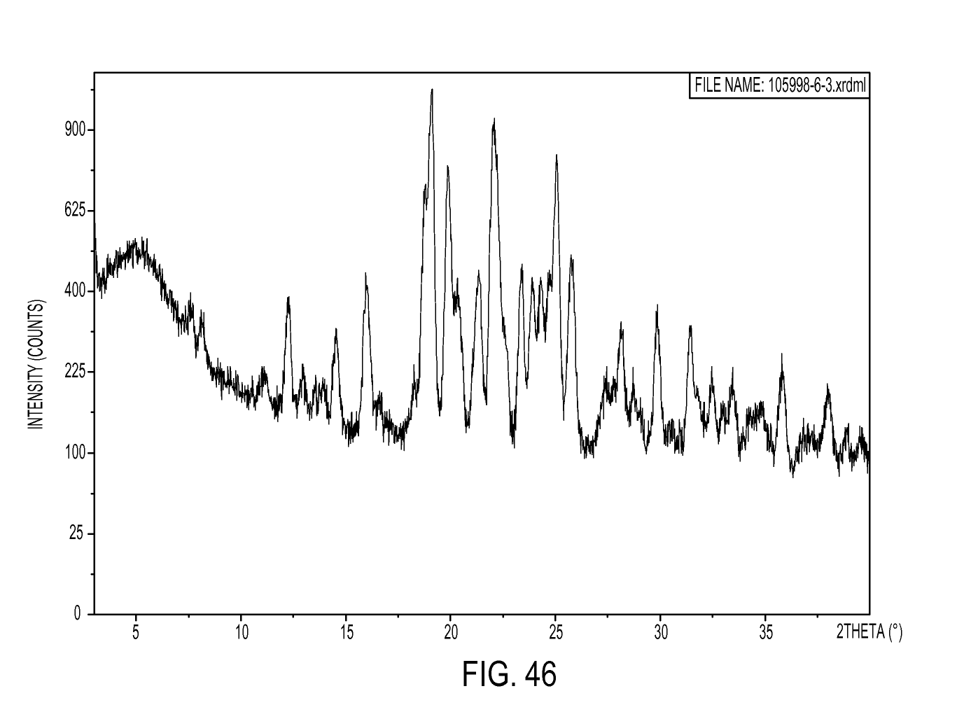

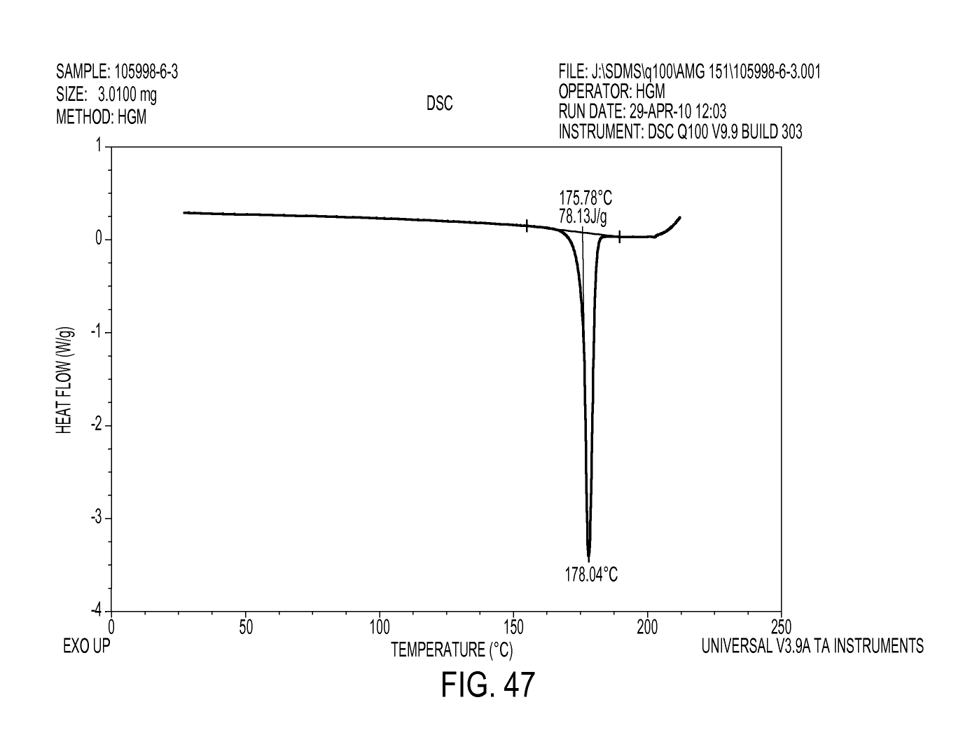

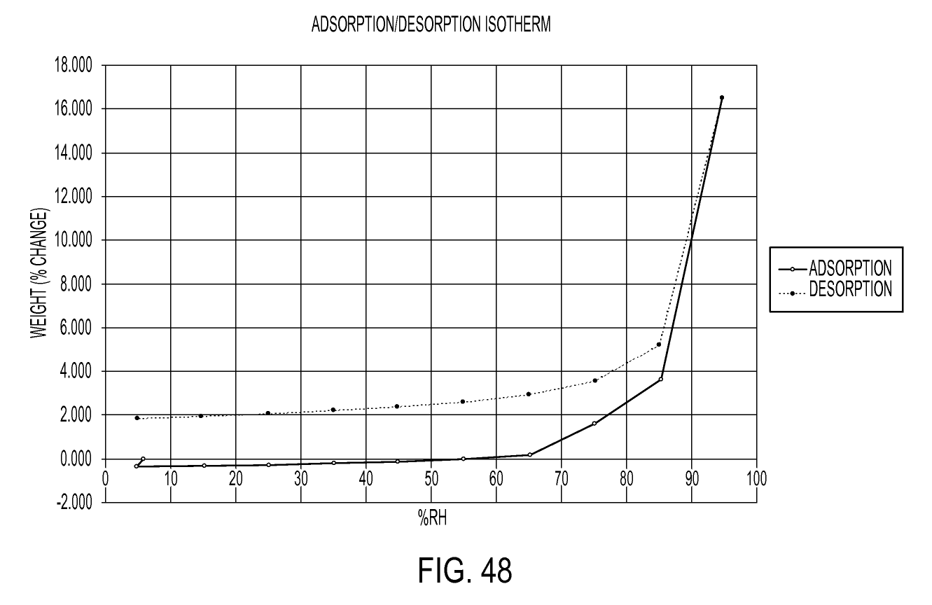

The MSA salt Form B material was generated, and a representative XRPD pattern indicates that the material is crystalline and is shown in FIG. 46. This phase was further characterized via DSC (FIG. 47) and the curve indicates a sharp endotherm around 178° C. (attributed to a melt). The vapor sorption curve is shown in FIG. 48 and indicates that the material is hygroscopic above 65% relative humidity and exhibits a 16.5% weight change as high as 95% relative humidity. Upon drying back down to ambient relative humidity, the material was found to retain approximately 2-3% of the sorbed water. The phase was found to have converted to Form C post experiment based on XRPD results. This phase appeared unstable as a function of relative humidity.

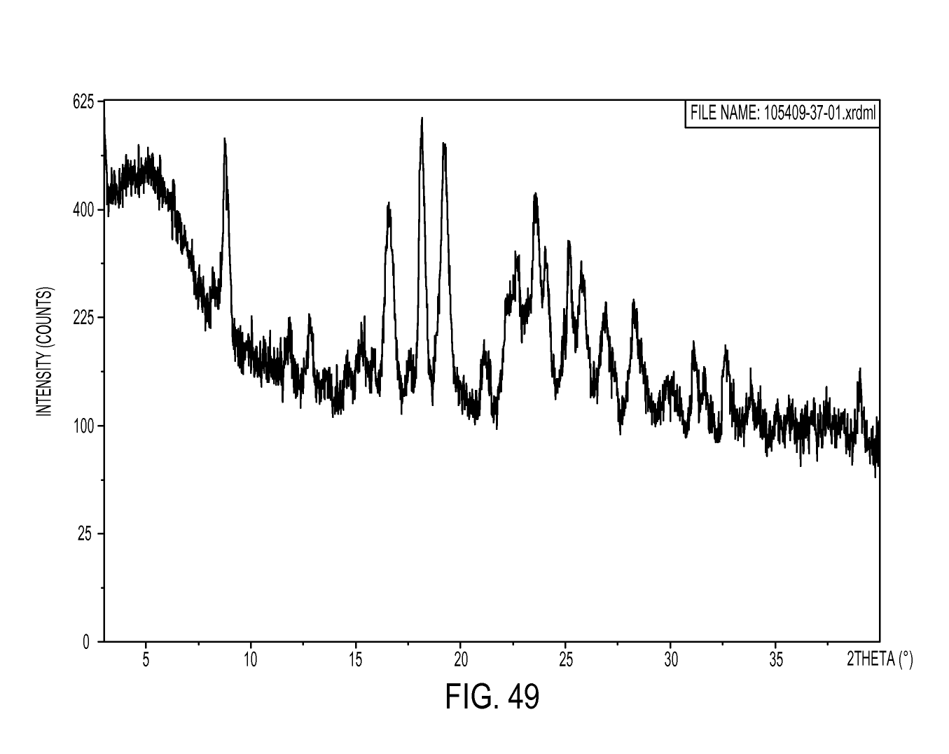

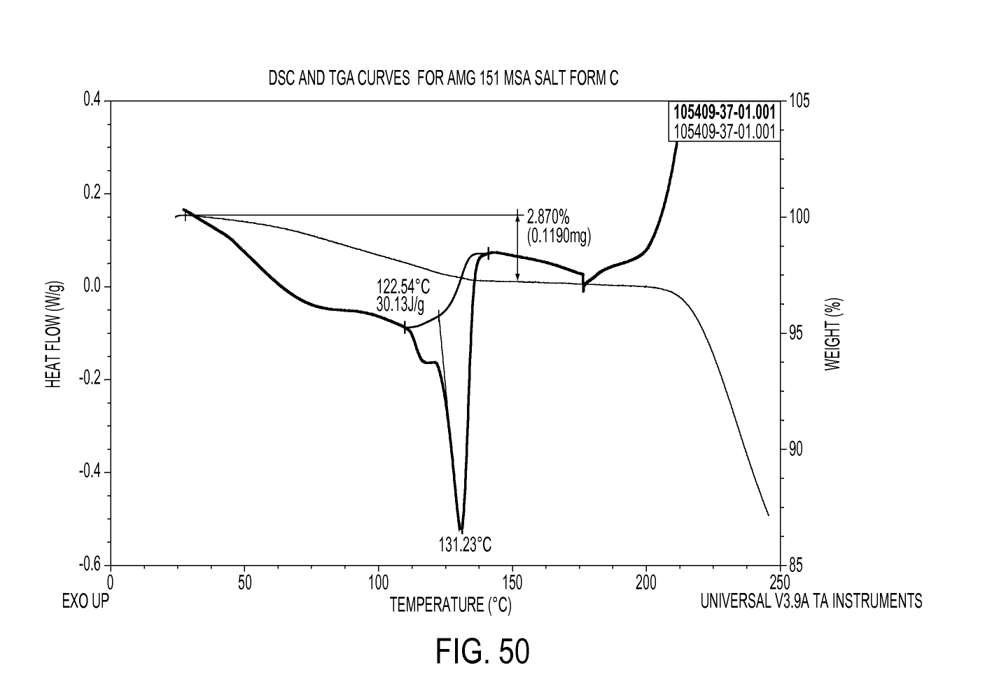

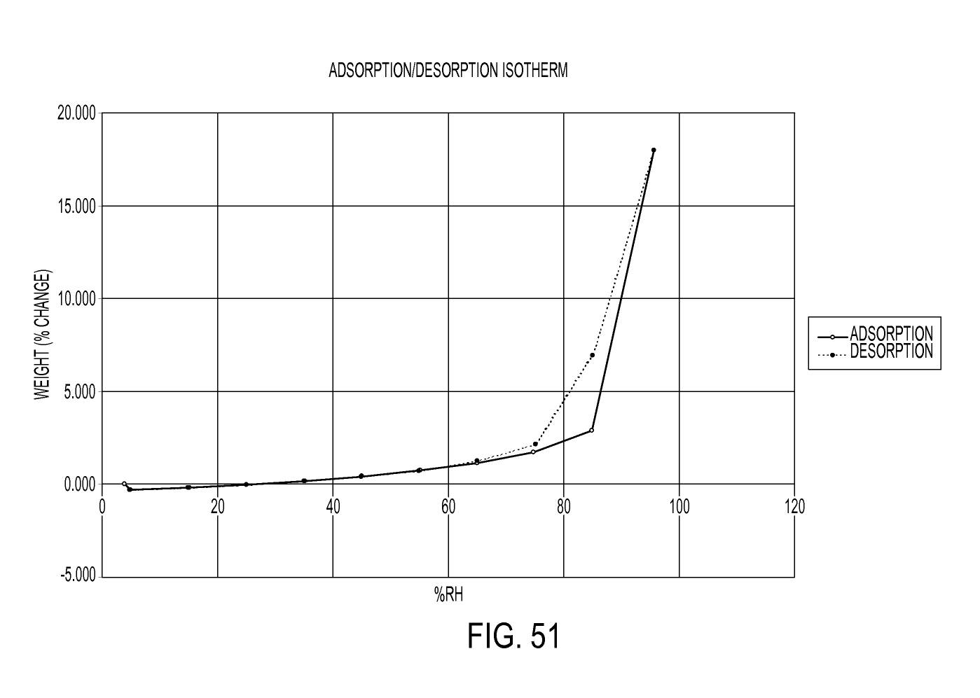

The MSA salt Form C material was generated by exposing Form B to elevated relative humidity, and a representative XRPD pattern indicates that the material is crystalline and is shown in FIG. 49. This phase was further characterized via DSC and TGA and the data are shown in FIG. 50. The DSC curve indicates a broad endotherm around 131° C. (attributed to desolvation with a melt). The vapor sorption curve is shown in FIG. 51 and indicates that the material is hygroscopic above 75% relative humidity and exhibits an 18% weight change as high as 95% relative humidity. Upon drying back down to ambient relative humidity, the material was found to retain approximately 2-3% of the sorbed water. The phase remained unchanged post experiment based on XRPD results. This phase appeared hygroscopic.

E. Succinate Co-crystal

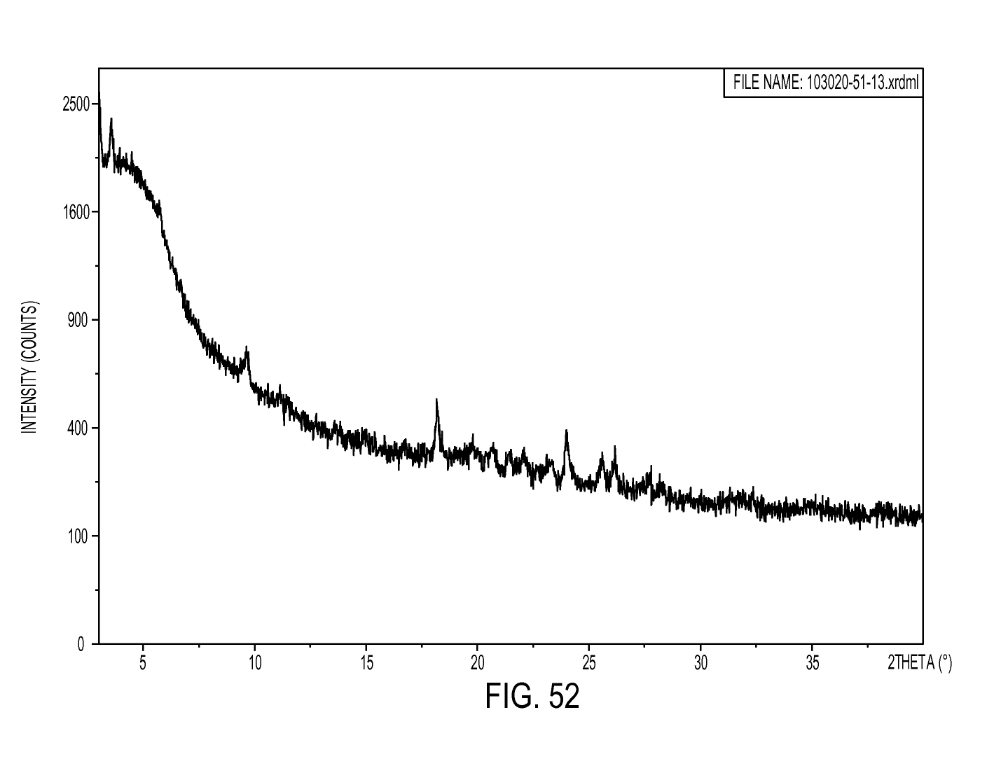

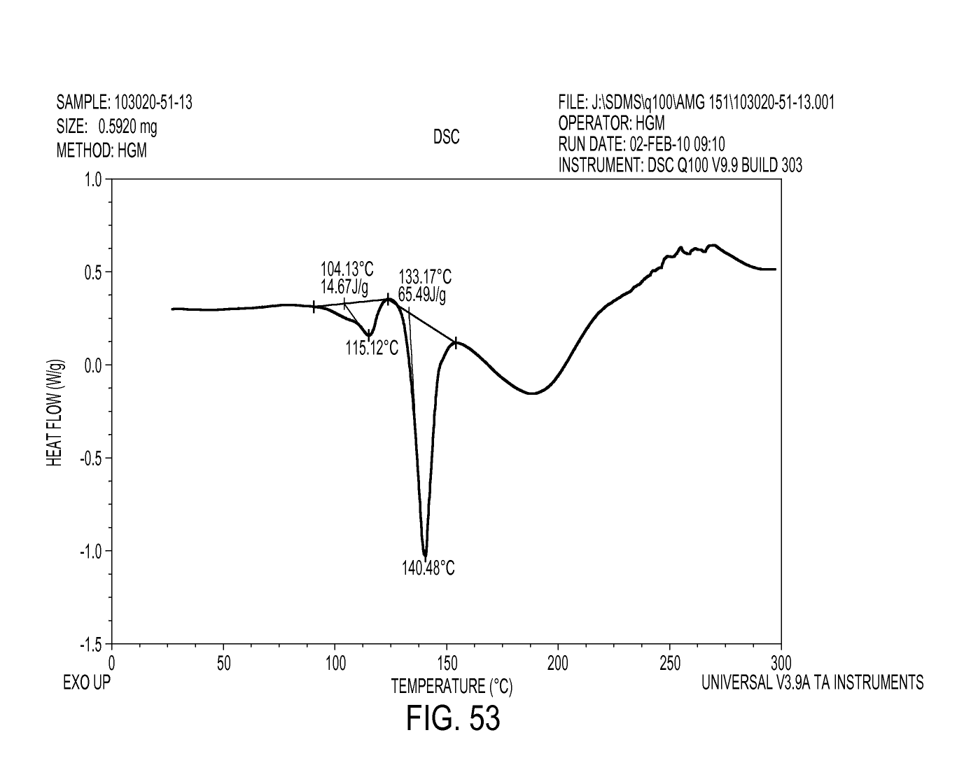

The succinate co-crystal Form A material was generated from acetone, and a representative XRPD pattern indicates that the material is crystalline and is shown in FIG. 52. This phase was further characterized via DSC (FIG. 53) and the curve indicates a broad endotherm around 115° C. and a sharp endotherm around 140° C. Further investigations are needed to understand the thermal events in the DSC curve.

F. Fumarate Co-crystal

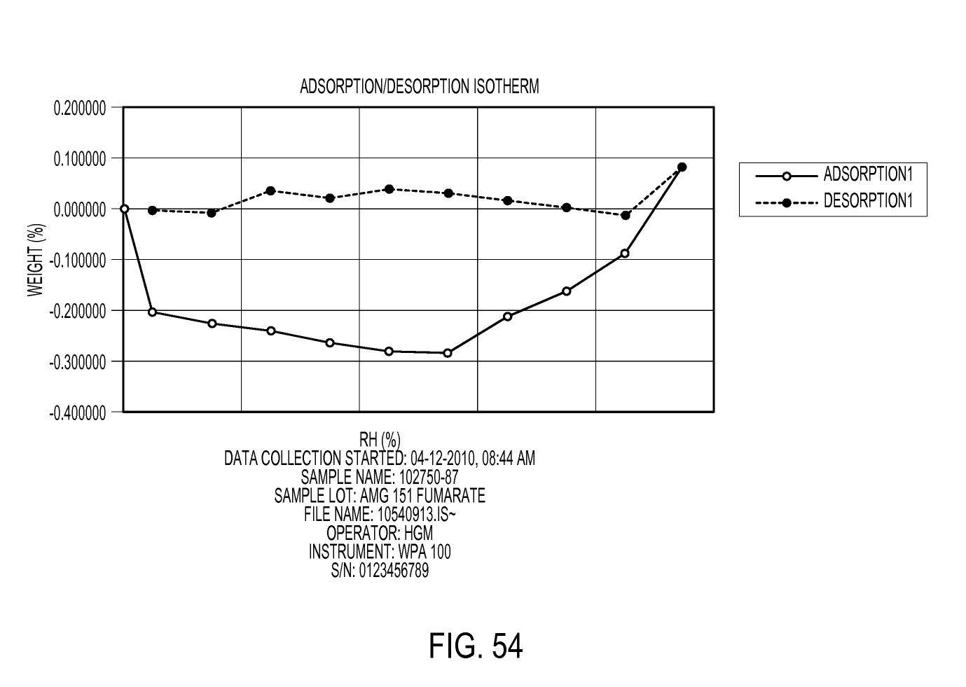

The fumarate co-crystal Form A material was generated from acetone MeOH and THF, and a representative XRPD pattern is shown in FIG. 7. The sample appears to be crystalline with needle morphology. This phase was further characterized via DSC and TGA and the data are shown in FIG. 8. The DSC curve indicates sharp endotherms around 186 and 197° C. The TGA curve shows no significant weight loss up to 175° C. indicating an unsolvated phase. The vapor sorption curve is shown in FIG. 54 and indicates that the material is non-hygroscopic and only exhibits a 0.075% weight change as high as 95% relative humidity. The phase remained unchanged post experiment based on XRPD results.

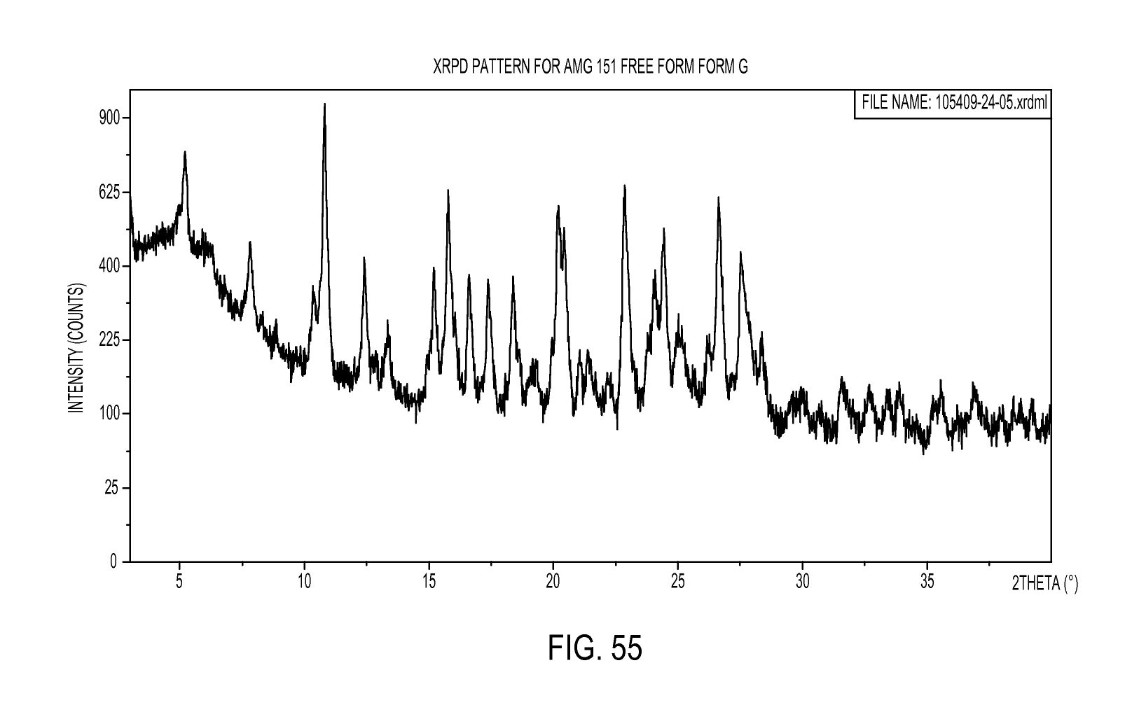

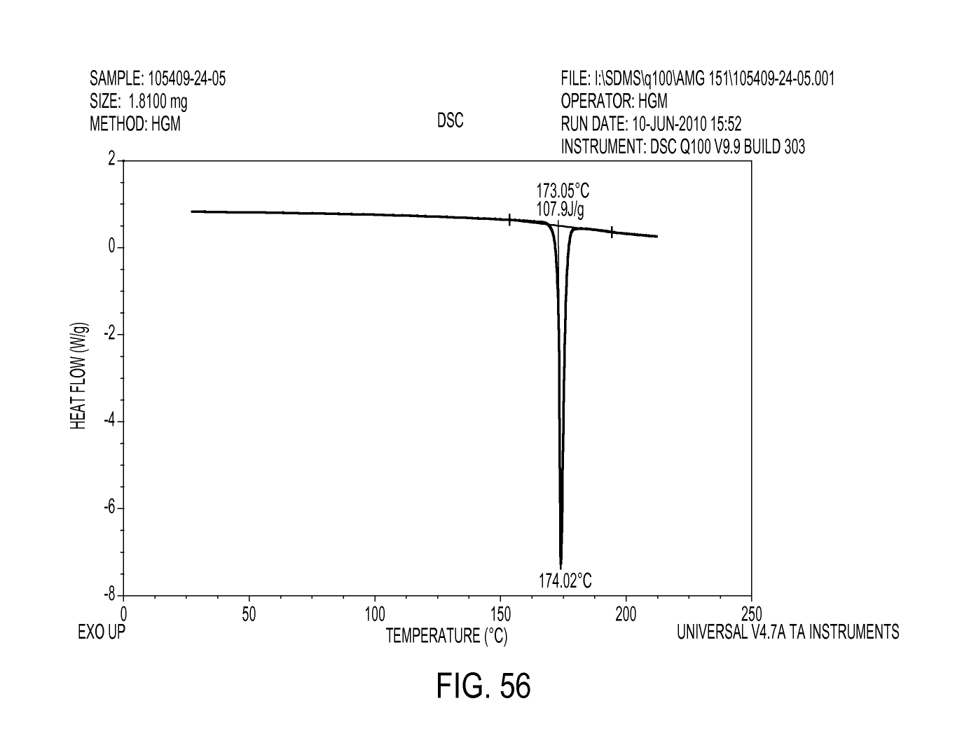

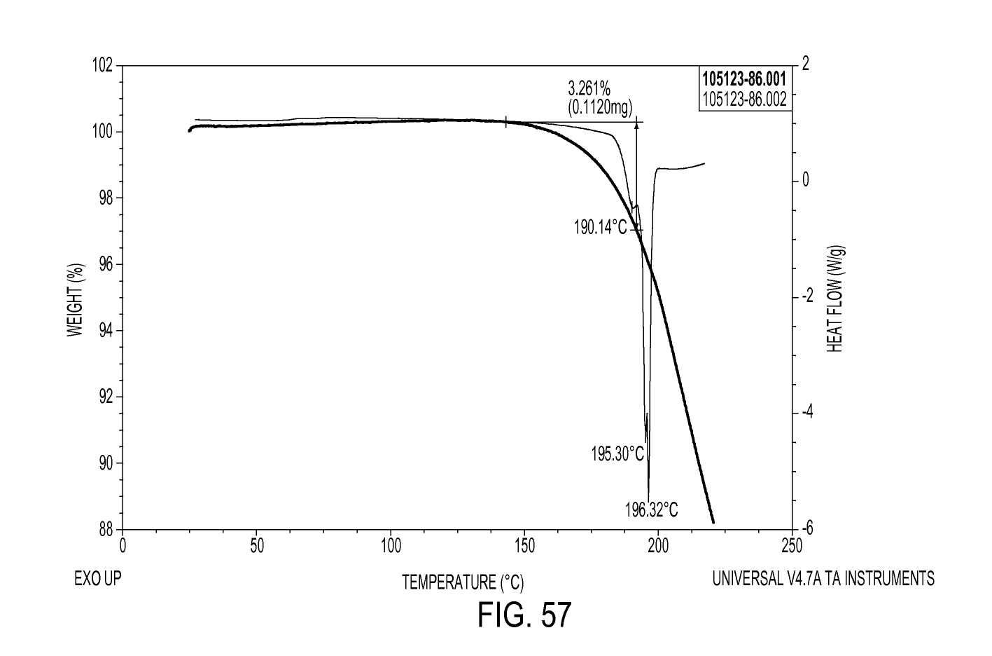

An investigation was needed to understand the two thermal events in the DSC curve. When a portion of the fumarate co-crystal was slurried at room temperature in MeOH, the resulting solids were found to have converted to a new low melting (174° C.) form of the free form (Form G) as verified by solution NMR. The XRPD pattern and DSC curve of the new free form (Form G) are shown is FIGS. 55 and 56, respectively. It was also determined that Form G of the free form would readily convert to Form F of the free form when stored at ambient conditions. Upon optimization of crystallization methods for the fumarate, the thermal characterization data of newer lots looked different (FIG. 57). The optimized lots were characterized by DSC curves with only two thermal events (typically around 190 and 196° C.). Furthermore, the first endotherm (which was initially thought to possibly be due to free form F/A contamination) was now showing up around 190° C., well above the melt of any known free form and well into decomposition temperatures when correlated with the TGA curve (3.2% weight loss through temperature range associated with the 190° C. endotherm). This appeared to indicate that the thermal events seen in the fumarate DSC curve may be due to decomposition of the sample and not a melt.

### Example 18

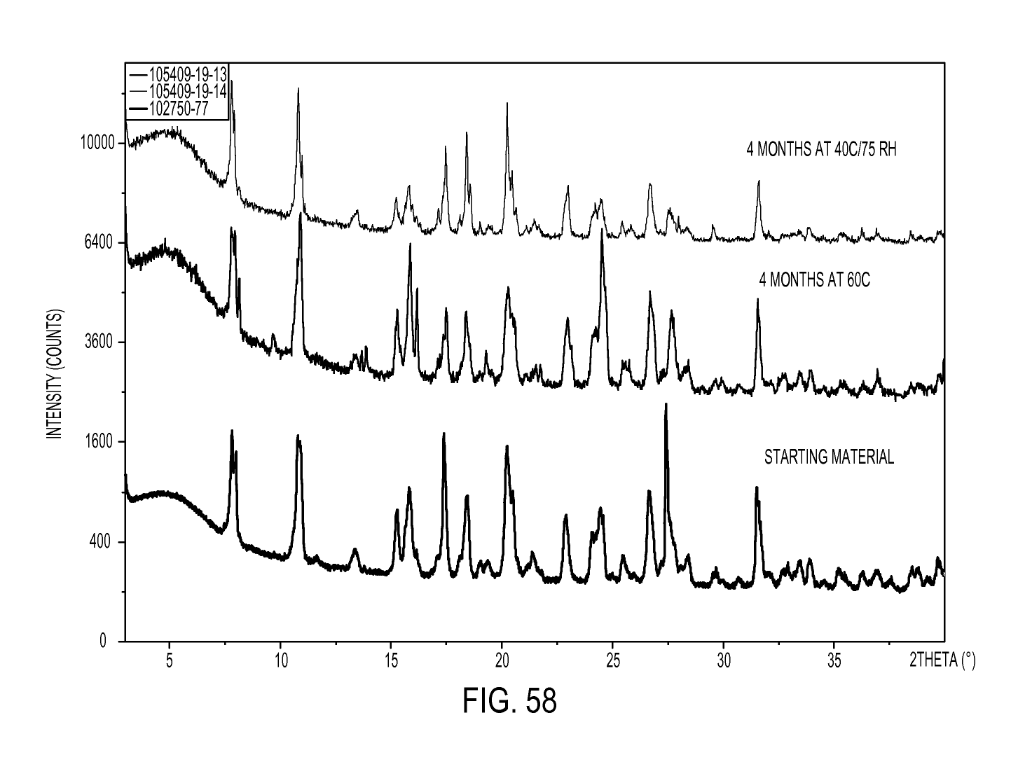

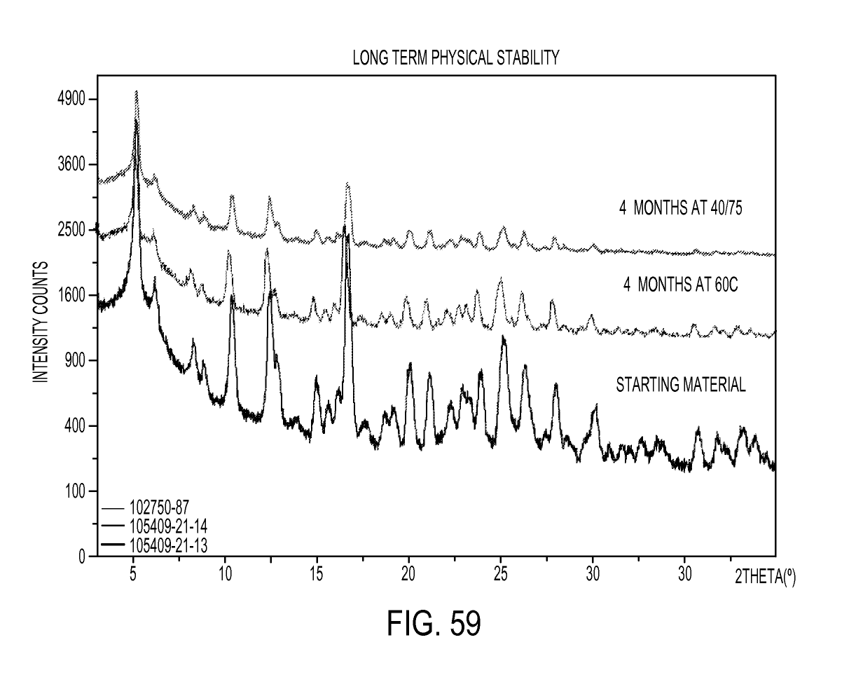

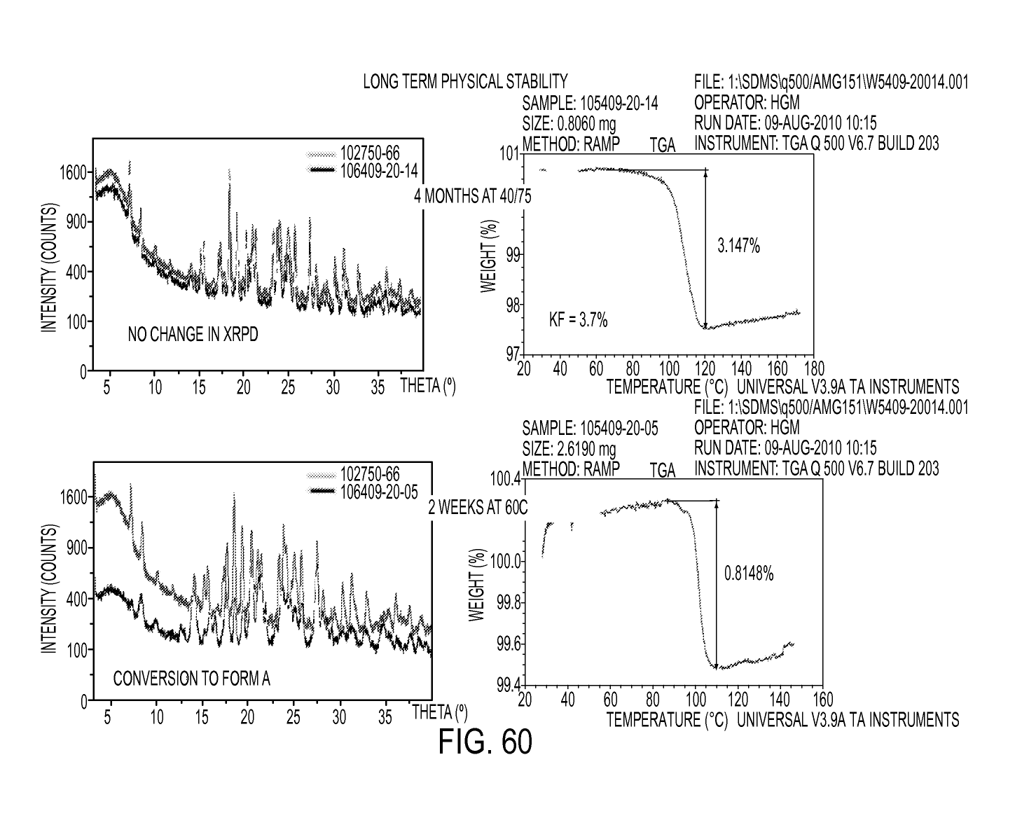

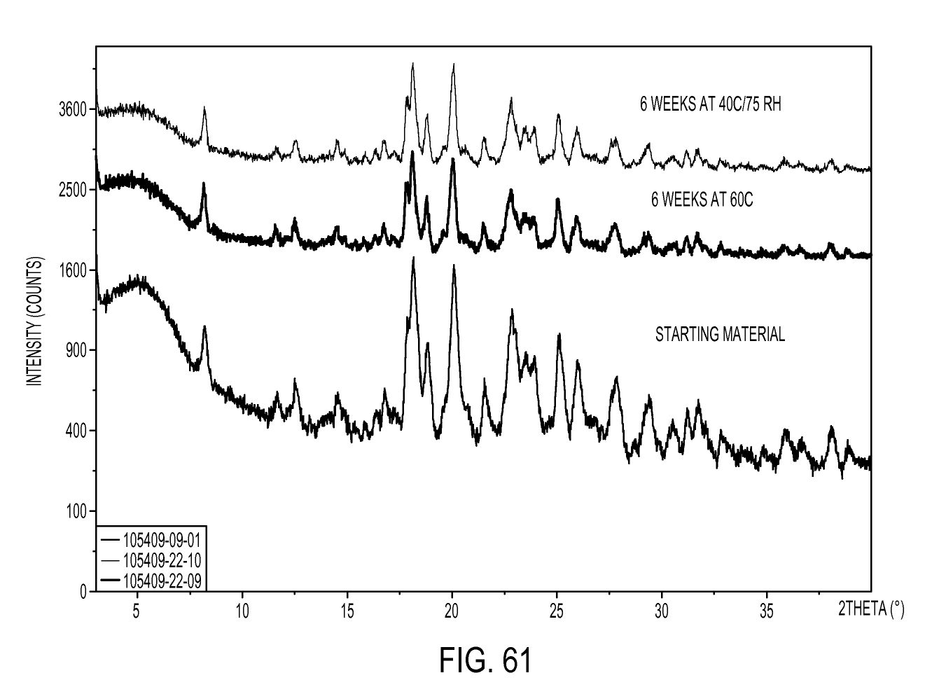

Chemical and physical stability was initiated on these forms and samples were stored at 60° C., 40° C./75% RH, 25° C./60% RH, and 5° C. The free base form F and the fumarate co-crystal were found to be chemically stable up to 4 months at 40° C./75% RH and 60° C. with no form change (FIGS. 58 and 59). The free base mono-hydrate was found to be chemically stable up to 4 months at 40° C./75% RH with no form change, but found to have converted to Form A after 2 weeks at 60° C. (FIG. 60). The phosphate salt was found to be stable up to 6 weeks at 40° C./75% RH and 60° C. with no form change (FIG. 61).

The free form Form F, free form hydrate and fumarate co-crystal were each made into three prototype formulations (each using different excipients per formulation and utilizing either a wet or dry blend) and were stressed at 25° C./60% RH and 40° C./75% RH for up to three months and then assayed.

### Example 19

### Acetic Acid Solvate Forms

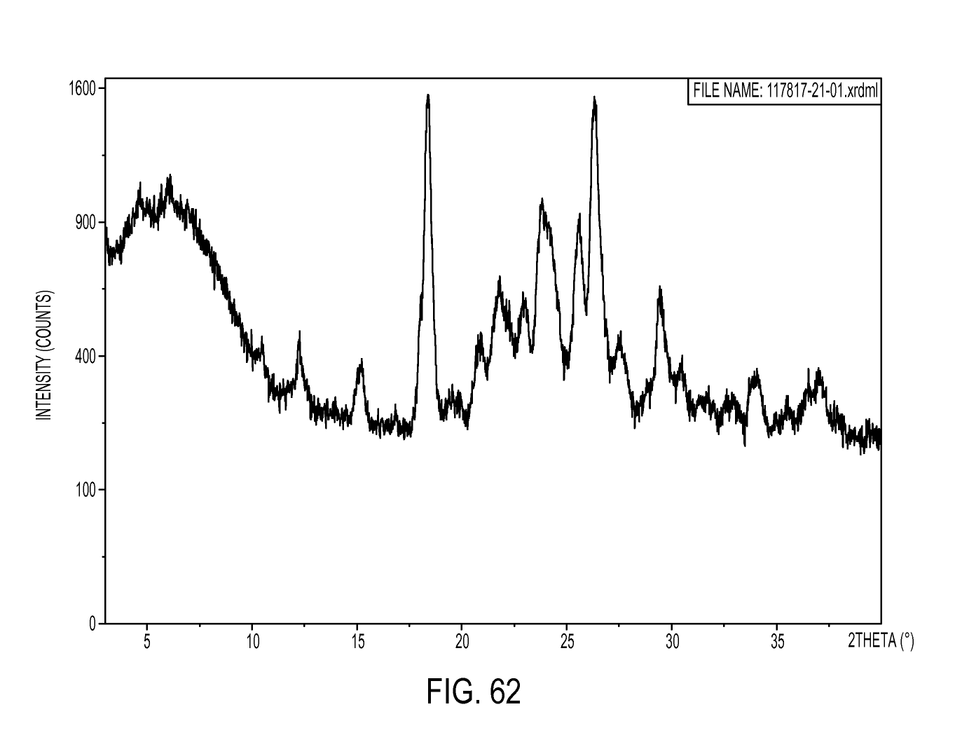

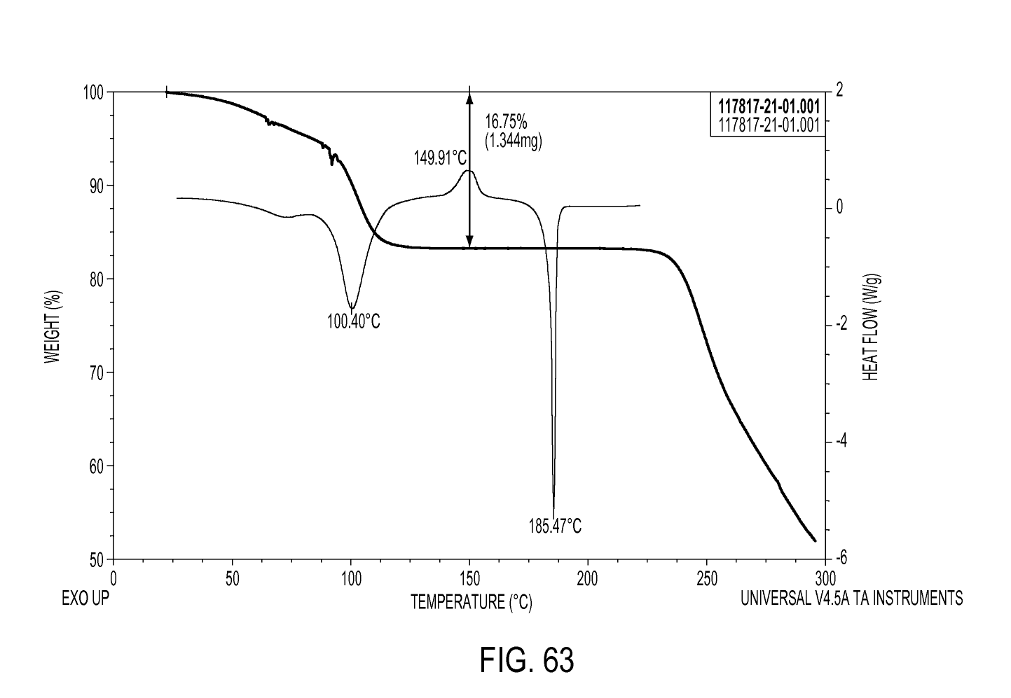

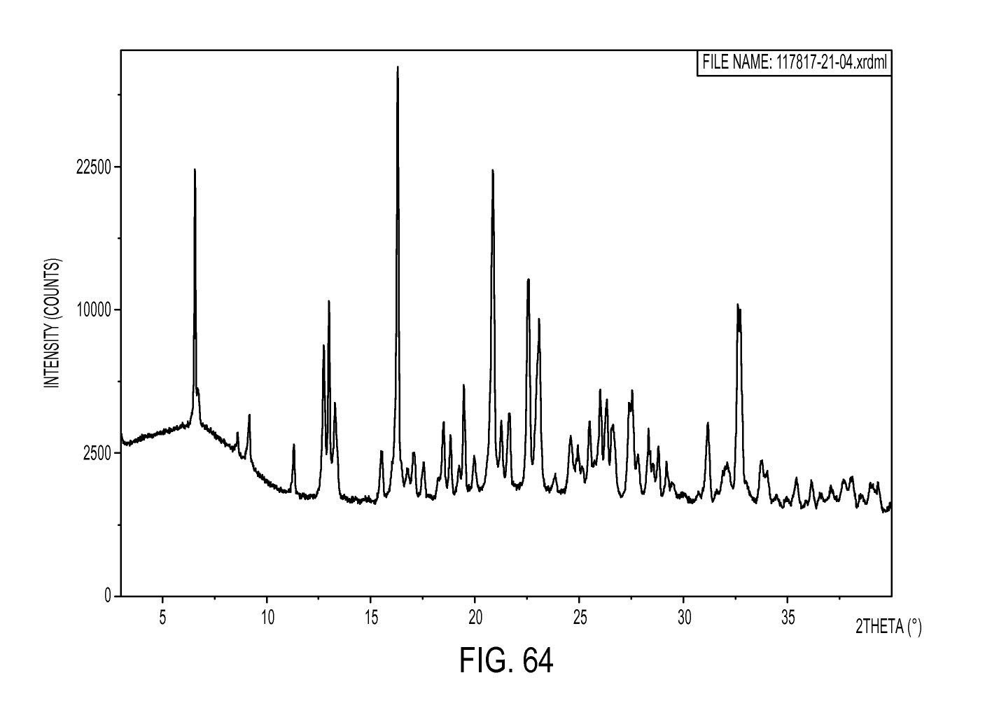

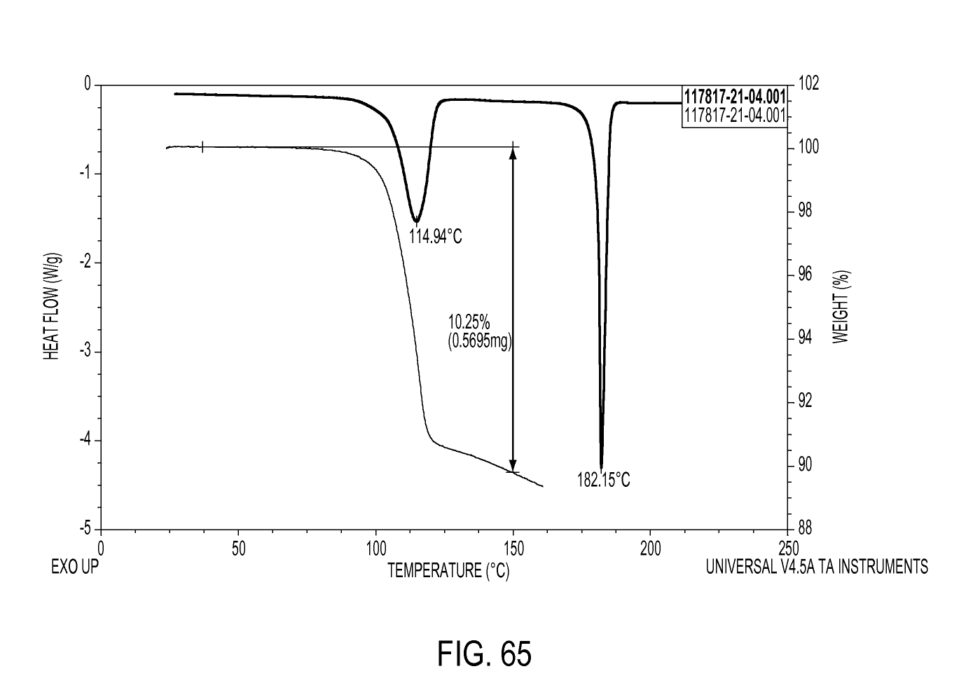

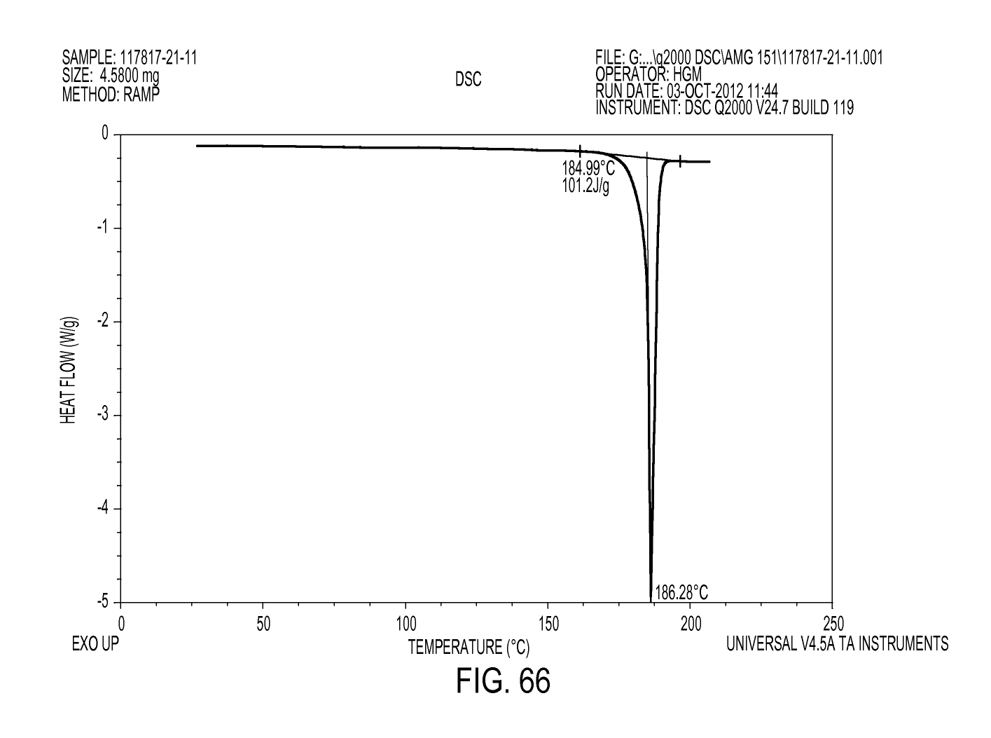

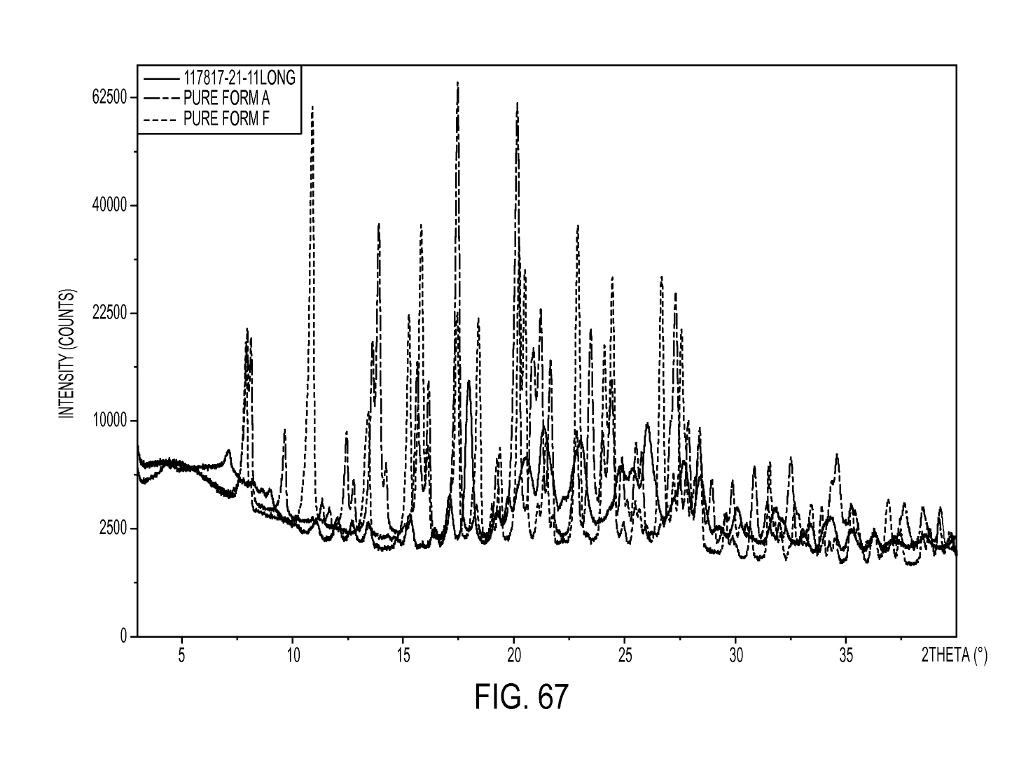

A crystalline AMG151 acetic acid solvate Form J was prepared by slurring AMG151 hydrate Form C in pure acetic acid at RT overnight. A crystalline AMG151 acetic acid solvate Form K was prepared by slurring AMG151 hydrate Form C in 40% water: 60% acetic acid at RT overnight. Representative XRPD pattern is shown in FIGS. 62 and 64, and DSC and TGA are shown in FIGS. 63 and 65. Upon heating Form J at 150° C. caused loss of acetic acid, forming a new Form I. DSC analysis is shown in FIG. 66. A melting point is observed at about 186° C. The acetic acid solvate (Form I) has a distinct XRPD pattern having unique reflections at around 11.9, 12.7, 13.3, 17.9 and 26.0 degrees 2θ as shown in FIG. 67.

### Example 20

### Free Base Form L

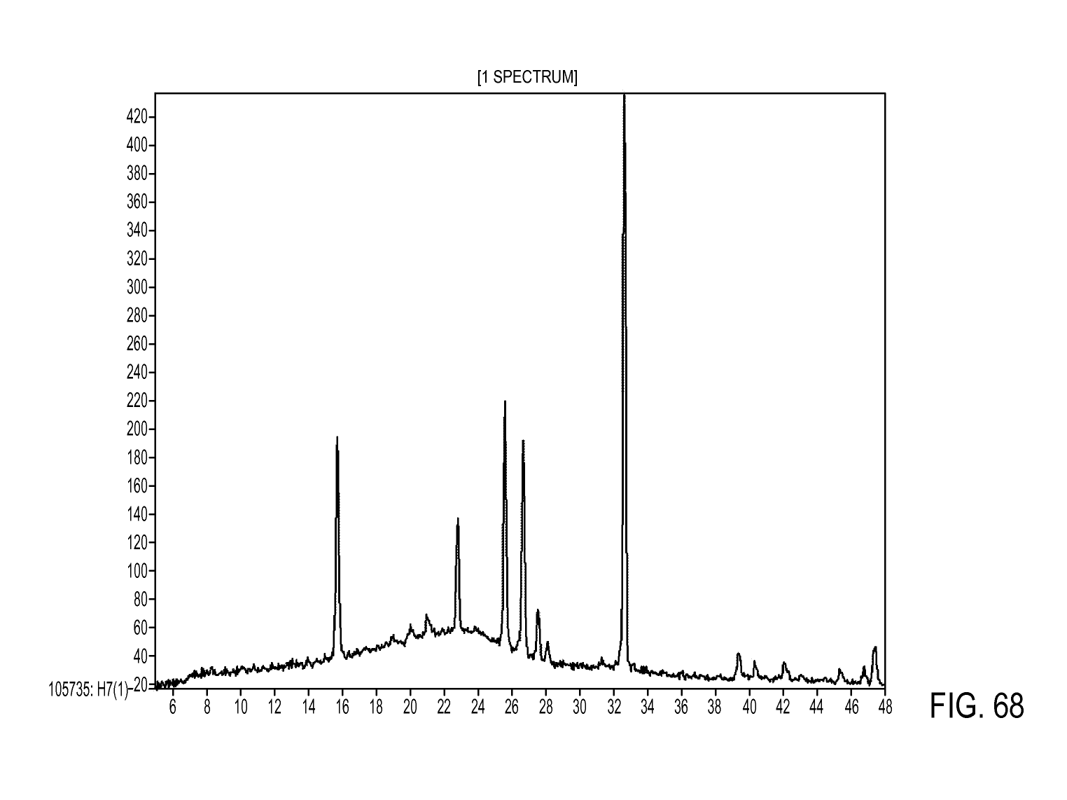

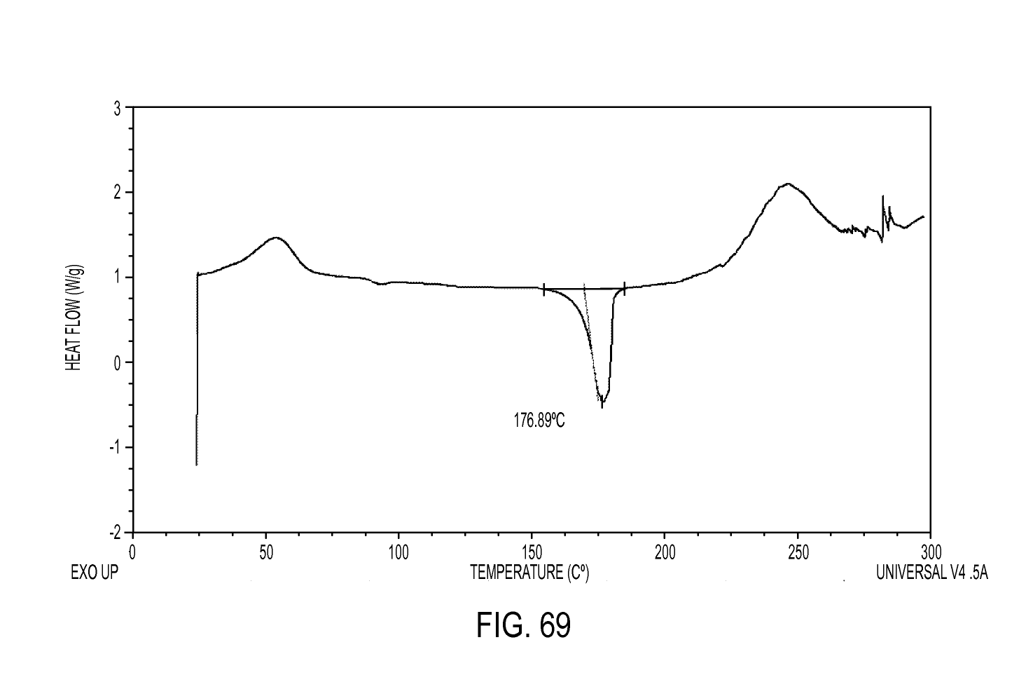

A form of the free base was isolated from a solution of either MEK/nitromethane, MEK/MeCN or EtOH/cyclohexane binary solvents under evaporation conditions. By DSC the form had an endotherm melt at 176.9° C. as shown in FIG. 69. A representative XRPD pattern is shown in FIG. 68.

### Example 21

### Free Base Form

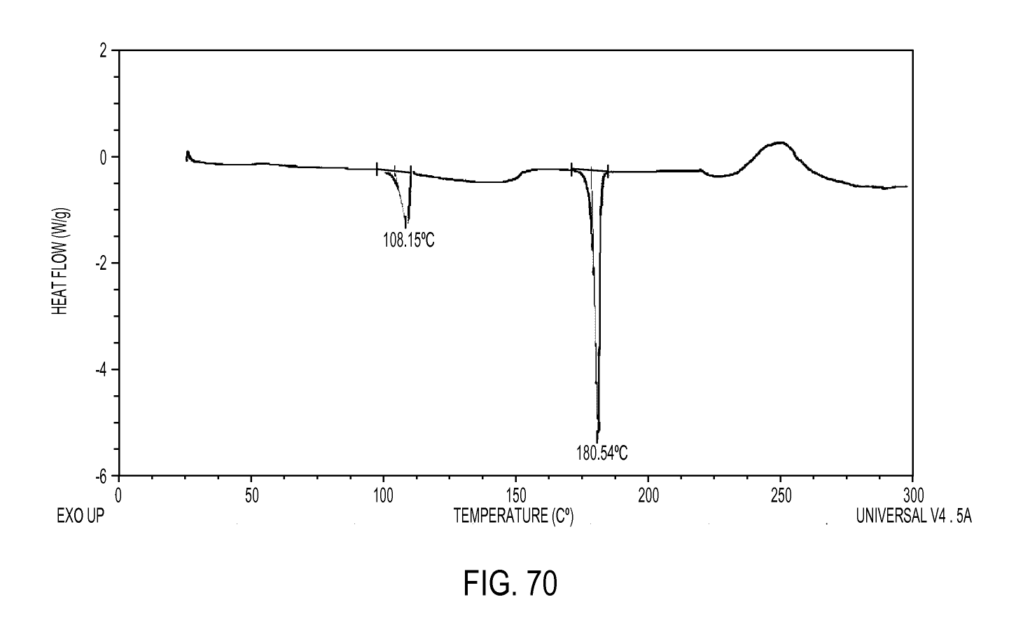

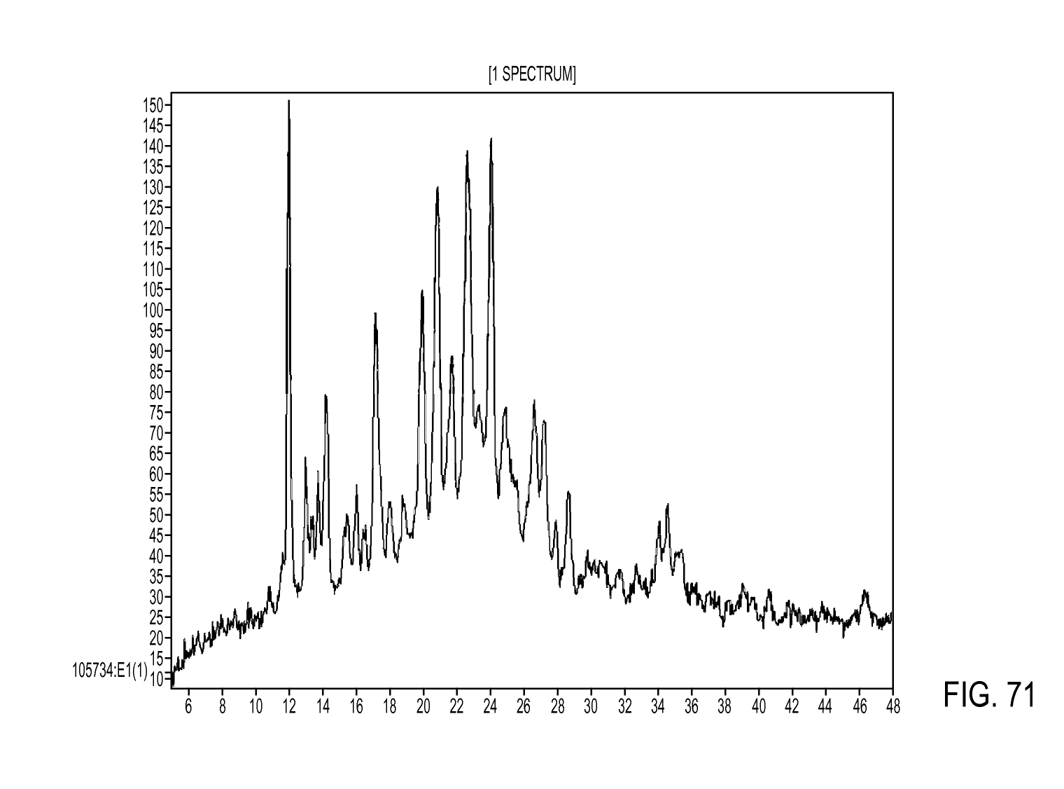

A form of the free base was isolated from a benzonitrile single solvent under slurry condition. By DSC the form had an endotherm peak at 108.2° C. and an endotherm melt at 180.5° C. as shown in FIG. 70. A representative XRPD pattern is shown in FIG. 71.

The foregoing is merely illustrative of the invention and is not intended to limit the invention to the disclosed forms. Variations and changes which are obvious to one skilled in the art are intended to be within the scope and nature of the invention which are defined in the appended claims. From the foregoing description, one skilled in the art can easily ascertain the essential characteristics of this invention, and without departing from the spirit and scope thereof, can make various changes and modifications of the invention to adapt it to various usages and conditions. Although this invention has been described with respect to specific embodiments, the details of these embodiments are not to be construed as limitations.

### Example 22

### Preparation of the Crystalline Polymorph of AMG151 Monohydrate, Form C, with Consistent Particle Size Distribution

A growth dominant, pH driven, reactive crystallization method of preparing AMG151 monohydrate, Form C, was developed and the factors with the greatest influence on the product particle size were determined. The robustness of the crystallization with respect to product particle size was improved through alternative buffer systems and pH monitoring. “Growth dominant” refers to crystal size and nucleation phenomena where the particles grow/nucleate on one another rather than nucleate to form “new” crystals.

The robustness of a sulfuric acid system with respect to particle size distribution (“PSD”) of product was evaluated. In this study, the strength of sulfuric acid was varied ±5%, and the equivalents of KOAc base added at the seed point were varied from 0.75 to 1.25, providing a variance in pH at the seed point of 1.7 to pH 2.1. The particle size distribution of the product using these varied crystallization parameters were measured with a Malvern Mastersizer 2000 instrument, using the wet dispersion method. All resulted in a consistent PSD with the exception of one outlier where agglomerated particles were observed (see Table 4). All robustness experiments used 3-5 wt % pin-milled seed. The growth kinetics of the sulfuric acid system appeared to be slower than with a hydrochloric acid system (data not shown for the HCl system). It was discovered that because the growth kinetics was slower with sulfuric acid, the particle size could be controlled to a smaller particle size distribution. Nucleation events were observed by focused beam reflectance measurement (FBRM) in all of the experiments with sulfuric acid system, even with a long (16 h) addition of base. Particle size robustness was observed using sulfuric acid.

In Table 4, d₁₀ means that 10% of the particles are less than the number listed; d₅₀ means 50% of the particles are less than the number listed; and d₉₀ means 90% of the particles are less than the number listed.

TABLE 4
Crystallization parameters and PSD of sulfuric acid experiments.

| pH @ | | | | |
|---|---|---|---|---|
| seed point | d₁₀ | d₅₀ | d₉₀ | Observations/comments |
| 2.04 | 4 μm | 15 μm | 43 μm | |
| 1.9 | 10 μm | 49 μm | 95 μm | Agglomerates observed |
| 1.75 | 6 μm | 21 μm | 61μm | Seed dissolved and batch |
| | | | | self-seeded |
| 1.94 | 4 μm | 15 μm | 47 μm | 1.0 eq of base added |
| 2.13 | 4 μm | 15 μm | 45 μm | 1.25 eq of base added |
| 1.92 | 3 μm | 18 μm | 58 μm | |
| 2.10 | 4 μm | 15 μm | 42 μm | |
| 1.73 | 4 μm | 19 μm | 51 μm | |
| 1.92 | 3 μm | 15 μm | 46 μm | 500 g scale (Example 23) |

### Example 23

### 500 g Scale Demonstration—Preparation of the Crystalline Polymorph of AMG151 Monohydrate, Form C, with Consistent Particle Size Distribution

The crystallization of AMG151 monohydrate using sulfuric acid was demonstrated on a 500 g scale in a 10 L vessel using the following general procedure:

| | |
|---|---|
| 1 | (1S)-1-[5-({3-[(2-Methylpyridin-3-yl)oxy]-5-(pyridin-2- |
| | ylsulfanyl)pyridin-2-yl}amino)-1,2,4-thiadiazol-3-yl]ethane- |
| | 1,2-diol (500 g) was added to 1N H₂SO₄ (1.5 equiv., 1500 mL) |
| | and stirred at 25 ± 5° C. for ≧5 h. |
| 2 | Water (500 mL) was added. |
| 3 | 1.0N Aqueous KOAc solution (1200 mL) was added over ≧30 |
| | minutes while maintaining a batch temperature of 25 ± |
| | 5° C. The pH was typically around 2. |
| 4 | (1S)-1-[5-({3-[(2-Methylpyridin-3-yl)oxy]-5-(pyridin-2- |
| | ylsulfanyl)pyridin-2-yl}amino)-1,2,4-thiadiazol-3-yl]ethane- |
| | 1,2-diol (14.18 g) was added to seed the crystallization. |
| 5 | 1.0N Aq KOAc (1800 mL) solution is added over ≧8 h while |
| | maintaining a batch temperature of 25 ± 5° C. The pH is |
| | typically around 2. |
| 6 | Batch was agitated at 25 ± 5° C. overnight. |
| 7 | Batch was filtered in a 3 L filter dryer. |
| 8 | Filter cake was washed with water (2 L). |
| 9 | Filter cake was dewatered with nitrogen |
| 10 | Filter cake was dried at 40° C. and reduced pressure (about 130 |
| | mbar) until the target of 3.5-3.8 wt % water was achieved. |
| 11 | Product was discharged from dryer. |

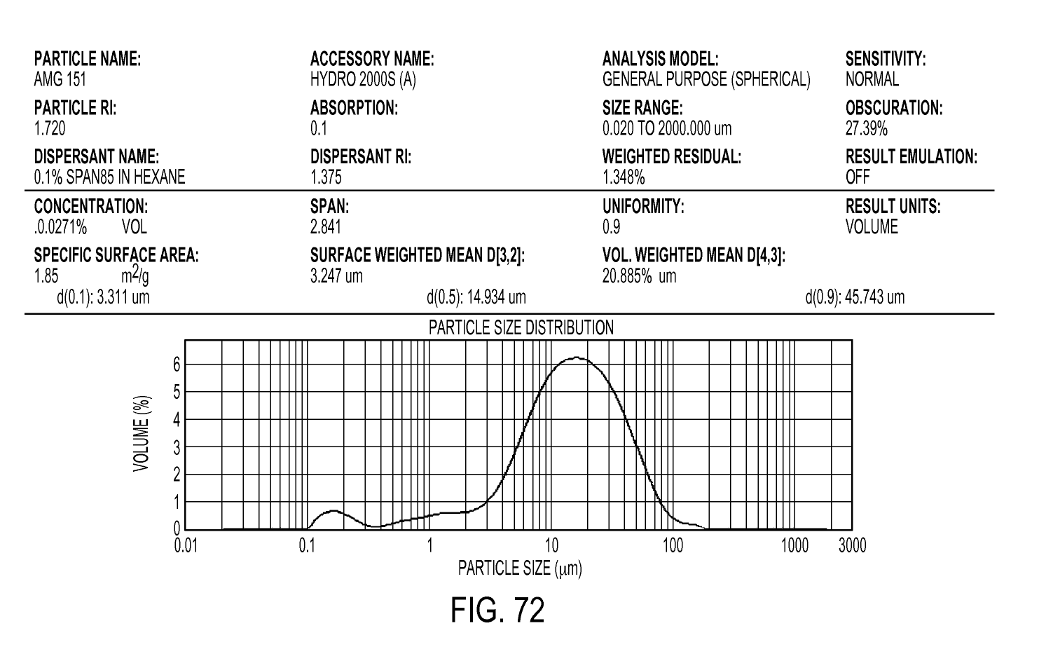

The crystallization was effected at pH 1.92, and the slurry was aged overnight to complete crystallization. The crystalline product was isolated via filtration and dried under a stream of nitrogen (467 g, 96% yield). Particle size distribution was measured with a Malvern Mastersizer 2000 instrument, using the wet dispersion method. See FIG. 72 and Table 4 for the resulting particle size data.

### Example 24

### Crystallization of the AMG151 Free Base Form C and Subsequent Polymorphic Transformation to Polymorphic Form a without Seeding

It was observed that larger scale productions of AMG151 free base resulted in both free base batches having increased levels of polymorphic Form F. In one batch, it was observed that during the addition of water, then ethanol followed by concentrated HCl that the mixture never became homogeneous and crystalline solids were observed. The solids were analyzed after polish filtration to be polymorphic AMG151 hydrate, Form C with some small amount of Form A (Scheme G). In another batch, the addition of water was quickly followed by ethanol and concentrated HCl which resulted in a homogeneous solution, although near to the completion of the polish filtration a little crystallization of solids was observed, presumably AMG151 hydrate Form C.

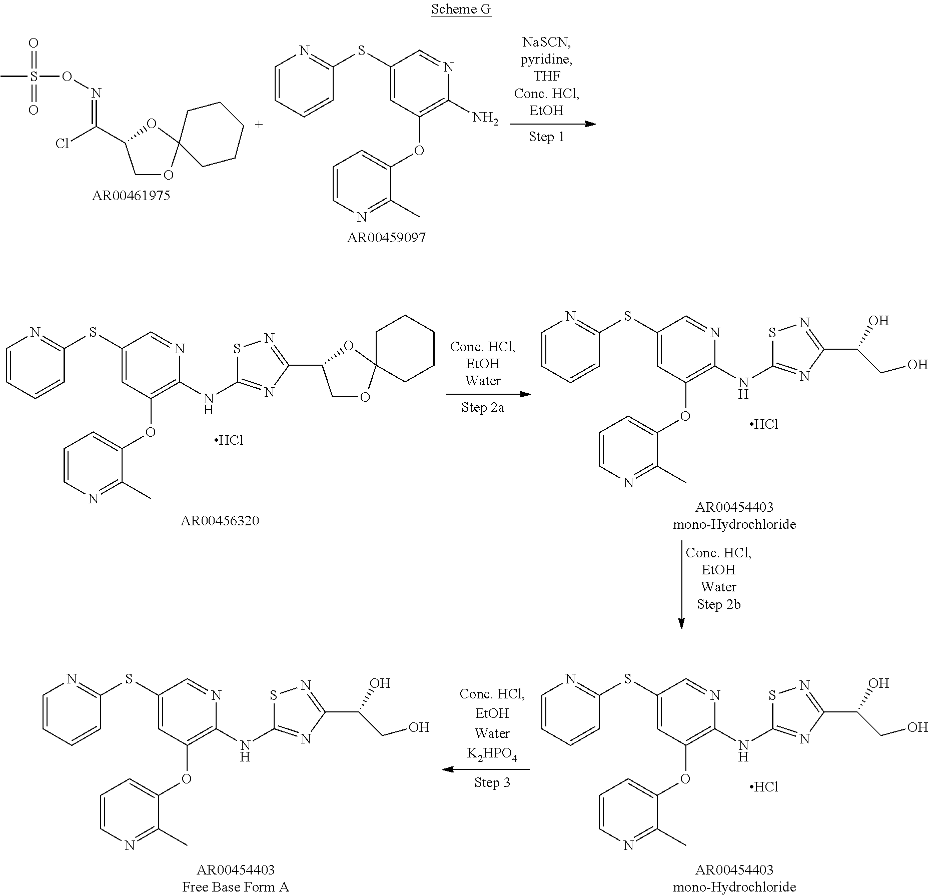

It was found that hydrate Form C could be converted to Form A at ambient temperature in both a 70:30 v/v and 75:25 v/v Water: EtOH solvent combinations at 20 mL/g. The possibility of crystallizing hydrate Form C and then proceeding with a solvent mediated polymorphic conversion to pure Form A was therefore explored. This would eliminate the need to seed the crystallization that has shown to produce varying amounts of Form A and F depending on the ratio of these two forms present in the seed crystals.

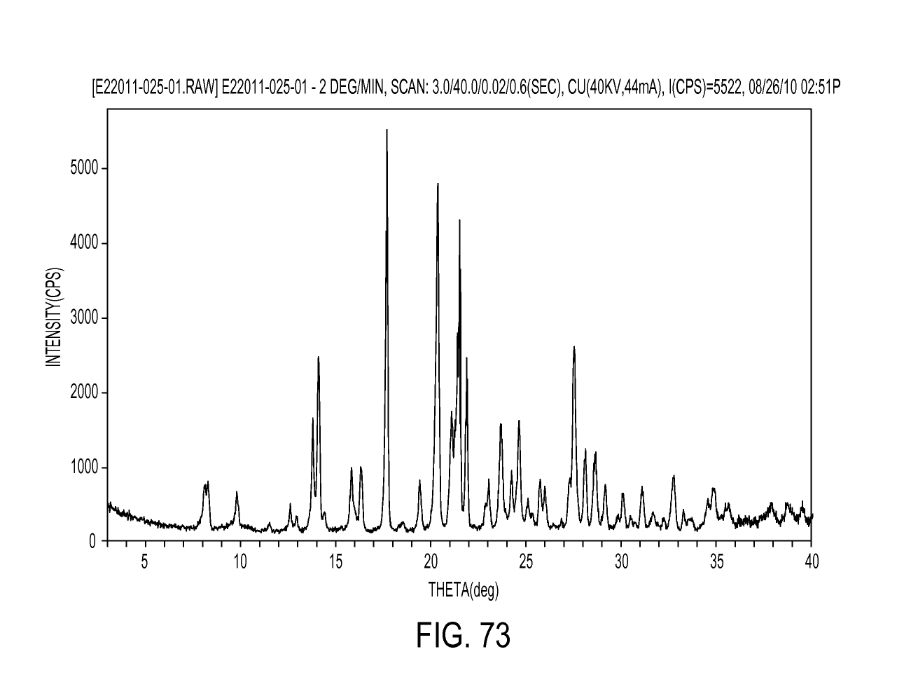

A first reaction vessel was charged with (S)-1-(5-(3-(2-methylpyridin-3-yloxy)-5-(pyridin-2-ylthio)pyridin-2-ylamino)-1,2,4-thiadiazol-3-yl)ethane-1,2-diol hydrochloride (AMG151, 8.0 g, 7.0 wt % Cl), followed by water (56 mL, 7.0 mL/g of AMG151), ethyl alcohol (absolute, 24 mL, 3.0 mL/g of AMG151), and 37% aqueous hydrochloric acid (0.16 g, 9 wt % chloride assay of AMG151). The reaction mixture was transferred to second reaction vessel with a polish filtration through GFF paper. The reaction was agitated at 20 to 30° C. (Note: The product slowly crystallized from the solution after 40 minutes and was continued to age over 3 hours providing a thick crystalline slurry that was confirmed as hydrate form C by XRPD.) A second reaction vessel was charged with potassium phosphate dibasic (4.1 g, 1.44 equivalents), followed by water (56 mL, 7.5 mL/g of AMG151) and agitated until homogeneous. Ethyl alcohol (absolute, 24 mL, 2.5 mL/g of AMG151) was then added. This mixture was polish filtered through GFF paper and slowly transferred into the first reactor containing the AMG151 hydrochloride over approximately 50 minutes. XRPD analysis confirmed Form C. (Note: pH=6.56, T=26.0° C. A pH >4.5 was chosen to avoid any potential degradation to the aminopyridine during the polymorphic transformation from Form C to Form A). The crystalline mixture was agitated at 25° C. for 17 hours. XRPD analysis confirmed Form C. The crystalline mixture was heated to 50° C. for 23 hours. XRPD analysis confirmed Form C. Ethyl alcohol (absolute, 14 mL) was added to provide a solvent ratio of water:EtOH 65:35 v/v. The crystalline mixture was agitated at 50° C. for 4 hours. XRPD analysis confirmed Form C. Ethyl alcohol (absolute, 14 mL) was added to provide a solvent ratio of water:EtOH 60:40 v/v. The crystalline mixture was agitated at 50° C. for 2 hours. XRPD analysis confirmed Form C with the presence of Form A. The crystalline mixture was agitated at 50° C. for 68 hours. XRPD analysis confirmed complete conversion of Form C to the desired crystalline polymorphic Form A. The crystalline mixture was cooled to ambient temperature and filtered. The wet cake was washed twice with a 60:40 v/v mixture of water: EtOH (10 mL, water 0.75 mL/g of AMG151 and ethyl alcohol 0.5 mL/g of AMG151). The wet cake was then dried in a vacuum oven at 55° C. and −22 mmHg with a nitrogen bleed to provide the desired product, (S)-1-(5-(3-(2-methylpyridin-3-yloxy)-5-(pyridin-2-ylthio)pyridin-2-ylamino)-1,2,4-thiadiazol-3-yL)ethane-1,2-diol polymorphic Form A (5.78 g). The XRPD pattern is shown in FIG. 73.

### CONCLUSION

This provides a process of preparing crystalline polymorph AMG151 free base substantially in the form of Form A, whereby no seeding is necessary. This process removes the concern over the quality of the seed (presence of Form F in the Form A seed crystals) which can result in crystal growth of Form F in addition to Form A, providing product with elevated levels of the Form F polymorph.

Adjusting the solvent ratio from an initial ratio of 70:30 v/v mixture of water:EtOH to 60:40 v/v mixture of water: EtOH presumably enabled improved solubility and hence provided the solvent mediated polymorphic transformation from hydrate Form C to anhydrous Form A.

The isolated yield may improve if the solvent ratio of Water: EtOH is adjusted back to approximately 70:30 v/v to 75:25 v/v by charging additional water back to the crystalline slurry prior to isolation.

All mentioned references, patents, applications and publications, are hereby incorporated by reference in their entirety, as if here written.

1. A crystalline polymorph of (1 S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol monohydrate, Form C.

2. The polymorph of claim 1, wherein said polymorph is characterized by XRPD diffraction peaks (2θ degrees) at about 6.9, 8.2, 18.2, 19.2, and 30.2.

3. The polymorph of claim 1 wherein said polymorph is characterized by having an endothermic peak onset at about 113° C. by differential scanning calorimetry.

4. The polymorph of claim 1, comprising about 3.2% to about 4.6% water.

5. A crystalline polymorph of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol, Form A, wherein said polymorph is characterized by XRPD diffraction peaks (2θ degrees) at about 9.6, 12.4, 19.9, 20.1, and 23.4.

6. The crystalline polymorph of claim 5, wherein said polymorph is substantially in the form of Form A.

7. A crystalline polymorph of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol, Form F, wherein said polymorph is characterized by XRPD diffraction peaks (2θ degrees) at about 10.8, 15.2, 15.8, 20.4, and 26.7.

8. A mixture of crystalline polymorph of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol, Form A and crystalline polymorph of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol, Form F.

9. The mixture according to claim 8, where Form A is characterized by XRPD diffraction peaks (2θ degrees) at about 9.6, 12.4, 19.9, 20.1, and 23.4, and Form F is characterized by XRPD diffraction peaks (2θ degrees) at about 10.8, 15.2, 15.8, 20.4, and 26.7.

10. The mixture according to claim 9, wherein said mixture comprises:
(a) about 0.1% by weight of Form A and 99.9% is Form F; or
(b) about 10% by weight of Form A and 90% of Form F; or
(c) about 20% by weight of Form A and 80% of Form F; or
(d) about 25% by weight of Form A and 75% of Form F; or
(e) about 30% by weight of Form A and 70% of Form F; or
(f) about 40% by weight of Form A and 60% of Form F; or
(g) about 50% by weight of Form A and 50% of Form F; or
(h) about 60% by weight of Form A and 40% of Form F; or
(i) about 75% by weight of Form A and 25% of Form F; or
(j) about 80% by weight of Form A and 20% of Form F; or
(k) about 90% by weight of Form A and 10% of Form F; or
(l) about 99.9% by weight of Form A and 0.1% of Form F.

11. A pharmaceutical composition comprising a crystalline polymorph according to claim 1 in a total dosage amount of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol and one or more pharmaceutically acceptable excipients.

12. A method of treating diabetes in a patient, comprising administering a therapeutically effective amount of a crystalline polymorph according to claim 1 to said patient in need thereof.

13. (canceled)

14. A process for preparing (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol, comprising:
treating 3-((2-methylpyridin-3-yl)oxy)-5-(pyridin-2-ylthio)pyridin-2-amine with a base and (S)-3-(2,2-dimethyl-1,3-dioxolan-4-yl)-5-tosyl-1,2,4-thiadiazole to form (S)-3-(2,2-dimethyl-1,3-dioxolan-4-yl)-N-(3-((2-methylpyridin-3-yl)oxy)-5-(pyridin-2-ylthio)pyridin-2-yl)-1,2,4-thiadiazol-5-amine; and
treating said (S)-3-(2,2-dimethyl-1,3-dioxolan-4-yl)-N-(3-((2-methylpyridin-3-yl)oxy)-5-(pyridin-2-ylthio)pyridin-2-yl)-1,2,4-thiadiazol-5-amine with an acid to provide (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol.

15. A process for preparing (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol, comprising:
treating 3-((2-methylpyridin-3-yl)oxy)-5-(pyridin-2-ylthio)pyridine 1-oxide and a compound having the formula: 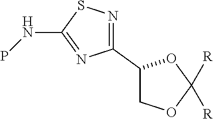
where P is a protecting group, and each R is an aryl or alkyl group, or the two R groups together form cycloalkyl ring, with a base and Tosyl halide, followed by the addition of an acid to provide (1 S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol.

16. A process for preparing (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol comprising:
reacting 2-chloro-3-((2-methylpyridin-3-yl)oxy)-5-(pyridin-2-ylthio)pyridine with (S)-3-(1,4-dioxaspiro[4.5]decan-2-yl)-1,2,4-thiadiazol-5-amine in the presence of a base to form to form (S)-3-(2,2-dimethyl-1,3-dioxolan-4-yl)-N-(3-((2-methylpyridin-3-yl)oxy)-5-(pyridin-2-ylthio)pyridin-2-yl)-1,2,4-thiadiazol-5-amine; and
treating (S)-3-(2,2-dimethyl-1,3-dioxolan-4-yl)-N-(3-((2-methylpyridin-3-yl)oxy)-5-(pyridin-2-ylthio)pyridin-2-yl)-1,2,4-thiadiazol-5-amine with acid to provide (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol.

17. A process for preparing crystalline polymorph of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol freebase, substantially in the form of Form A according to claim 6, comprising:
combining (1 S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol hydrochloride with water, ethyl alcohol and 37% aqueous hydrochloric acid to provide (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol hydrate, Form C;
adding K₂HPO₄, water and ethyl alcohol to the acidic solution to adjust the pH and further adding ethanol to the solution containing AMG151 hydrate, Form C until the ratio of water:ethanol is 60:40; and
stirring the solution at about 50° C. to provide (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol freebase, substantially in the form of Form A.

18. A process for preparing the crystalline polymorph of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol hydrochloride, substantially in the form of Form C according to claim 1, comprising:
combining (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol hydrochloride with water, ethyl alcohol and 37% aqueous hydrochloric acid; and
adding K₂HPO₄, water and ethyl alcohol to the acidic solution to provide the crystalline polymorph of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol hydrate, substantially in the form of Form C.

19. A process for preparing (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol monohydrate, Form C according to claim 1 with consistent particle size distribution, comprising:
combining (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol with a 1N aqueous sulfuric acid solution at ambient temperature;
adding water to said acidic solution; adding a 1.0 N aqueous potassium acetate solution to said acidic solution at ambient temperature to adjust the pH of the acidic solution to between 1.7 and 2.1;
seeding said acidic solution with (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol monohydrate, Form C when the pH of the acidic solution is between 1.7 and 2.1;
and allowing said Form C to crystallize from said solution.

20. The process of claim 19, wherein about 2-3 weight percent of said seed is added.

21. The process of claim 20, wherein said seed is pin-milled (1 S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol monohydrate, Form C.

22. The process according to claim 21, wherein said process provides (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol monohydrate, Form C having a particle size d₉₀ less than 20 μm.

23. The process according to claim 21, wherein said process provides (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol monohydrate, Form C having a particle size d₉₀ less than 50 μm.

24. The process according to claim 21, wherein said process provides (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol monohydrate, Form C having a particle size d₉₀ less than 100 μm.

25. The process according to claim 21, wherein said process provides (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol monohydrate, Form C having a particle size d₉₀ less than 200 μm.

26. The process according to claim 21, wherein said process provides (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol monohydrate, Form C, having a particle size distribution profile of: d₁₀ between about 3-6 μm, d₅₀ between about 15 and 21 μm, and d₉₀ between about 42 and 61 μm.

27. A pharmaceutical composition comprising a crystalline polymorph of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol, Form A according to claim 5, and one or more pharmaceutically acceptable excipients.

28. A pharmaceutical composition comprising a crystalline polymorph of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol, Form F, according to claim 7, and one or more pharmaceutically acceptable excipients.

29. A method of treating diabetes in a patient, comprising administering a therapeutically effective amount of a crystalline polymorph of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol, Form A, according to claim 5 to said patient in need thereof.

30. A method of treating diabetes in a patient, comprising administering a therapeutically effective amount of a crystalline polymorph of (1S)-1-[5-({3-[(2-methylpyridin-3-yl)oxy]-5-(pyridin-2-ylsulfanyl)pyridin-2-yl}amino)-1,2,4-thiadiazol-3-yl]ethane-1,2-diol, Form F, according to claim 7 to said patient in need thereof.
